(19) United States
(12) Patent Application Publication
(10) Pub. No.: US 2006/0234235 A1
(43) Pub. Date: Oct. 19, 2006

(54) Methods and compositions for the diagnosis of neuroendocrine lung cancer

(75) Inventors: Curtis C. Harris, Garrett Park, MD (US); Ping He, Bethesda, MD (US); Lyuba Varticovski, Bethesda, MD (US); Willliam D. Travis, Kensington, MD (US)

Correspondence Address: BERENATO, WHITE & STAVISH, LLC, 6550 ROCK SPRING DR., SUITE 240, BETHESDA, MD, 20817, US

(73) Assignee: The United States of America, as represented by the Secretary, dept of Health and human services, Rockville, MD (US)

(21) Appl. No.: 10/533,459

(22) PCT Filed: Nov. 3, 2003
(86) PCT No.: PCT/US03/34787

§ 371 Date: May 2, 2005

Related U.S. Application Data
(60) Provisional application No. 60/423,380, filed on Nov. 4, 2002.

(51) Int. Cl. C12Q 1/68; C12P 19/34; C12M 1/34

(52) U.S. Cl. 435/6; 435/91.2; 435/287.2

(57) ABSTRACT

This invention relates to methods and compositions for the diagnosis of neuroendocrine lung cancers. In particular, the invention concerns the use of cDNA microarrays to facilitate the differential diagnosis of neuroendocrine tumor types.

### STATEMENT OF GOVERNMENTAL INTEREST

This invention was funded by NCI Intramural Research Program CCR at the National Institutes of Health. The United States Government has certain rights to this invention.

### FIELD OF THE INVENTION

This invention relates to methods and compositions for the diagnosis of neuroendocrine lung cancers. In particular, the invention concerns the use of cDNA microarrays to facilitate the differential diagnosis of neuroendocrine tumor types.

### BACKGROUND OF THE INVENTION

The mammalian neuroendocrine system is a dispersed organ system that consists of cells found in multiple different organs. The cells of the neuroendocrine system function in certain ways like nerve cells and in other ways like cells of the endocrine (hormone-producing) glands. The neuroendocrine cells of the lung are of particular significance; they help control airflow and blood flow in the lungs and may help control growth of other types of lung cells.

In some instances, neuroendocrine cells escape from normal cellular control and become malignant, resulting in neuroendocrine tumors. Four clinically distinct types of neuroendocrine tumors have been described: small cell lung cancer (SCLC), large cell neuroendocrine carcinoma (LCNEC), typical carcinoid (TC) tumors and atypical carcinoid (AC) tumors. SCLC is the most serious type of neuroendocrine lung tumor (LCNEC), and is among the most rapidly growing and spreading of all cancers. Large cell neuroendocrine carcinoma, typical carcinoid and atypical carcinoid tumors are rare forms of cancers. Whereas SCLC accounts for 15-25% of total pulmonary malignancies, large cell neuroendocrine carcinoma, typical carcinoid and atypical carcinoid tumors collectively account for only 3-5% of total pulmonary malignancies. Accurate diagnosis of neuroendocrine carcinoma is important since the different tumor types are associated with markedly different survival rates. Small Cell Lung Cancers are associated with 5 and 10 year survival rates of only 9% and 5%, respectively. Large Cell Neuroendocrine Carcinoma presently exhibit 27% and 9%, 5 and 10 year survival rates. Atypical Carcinoid Tumors are associated with 5 and 10 year survival rates of 56% and 35%, respectively. In contrast, Typical Carcinoid Tumors are found to have 5 and 10 year survival rates of nearly 90%

Neuroendocrine tumors are reviewed by Gould, V. E. et al. (2000) “EPITHELIAL TUMORS OF THE LUNG” Chest Surg Clin Am 10:709-28, by DeLellis, R. A. (1997) “PROLIFERATION MARKERS IN NEUROENDOCRINE TUMORS: USEFUL OR USELESS? A CRITICAL REAPPRAISAL” Verh Dtsch Ges Pathol. 81:53-61, by Travis, W. D. et al. (1991) “NEUROENDOCRINE TUMORS OF THE LUNG WITH PROPOSED CRITERIA FOR LARGE-CELL NEUROENDOCRINE CARCINOMA. AN ULTRASTRUCTURAL, IMMUNOHISTOCHEMICAL, AND FLOW CYTOMETRIC STUDY OF 35 CASES” Am J Surg Pathol 15:529-53, by Cerilli, L. A. et al. (2001) “NEUROENDOCRINE NEOPLASMS OF THE LUNG” Am J Clin Pathol 116:S65-96; by Arrigoni, M. G., et al. (1972) “ATYPICAL CARCINOID TUMORS OF THE LUNG,” J Thorac Cardiovasc Surg 64:413-421; by Warren, W. H. et al. (1988) “WELL DIFFERENTIATED AND SMALL CELL NEUROENDOCRINE CARCINOMAS OF THE LUNG: TWO RELATED BUT DISTINCT CLINICOPATHOLOGIC ENTITIES,” Virchows Arch B cell Pathol 55:299-310; by Kramer, R. (1930) “ADENOMA OF BRONCHUS,” Ann Otol Rhinol Laryngol 39:689, and by Mark, E. J. et al. (1985) “PERIPHERAL SMALL CELL CARCINOMA OF THE LUNG RESEMBLING CARCINOID TUMOR,” Arch Pathol Lab Med 109:263-269.

Unfortunately, all neuroendocrine tumors have similar morphologic appearances and exhibit similar immunohistochemical staining. Thus, a significant difficulty presently exists in accurately distinguishing between the different types of neuroendocrine tumors. Such diagnosis is still “decisively” based on light-microscopic evaluations of tissue samples for the number of cells involved in mitosis. Other than clinical stage at presentation, mitotic count is currently the sole independent histologic predictor of prognosis (Junker, K. et al. (2000) “PATHOLOGY OF SMALL-CELL LUNG CANCER,” J Cancer Res Clin Oncol. 126:361-8; Franklin, Wash. (2000) “PATHOLOGY OF LUNG CANCER” J Thorac Imaging. 15:3-12; Chyczewski, L. et al. (2001) “MORPHOLOGICAL ASPECTS OF CARCINOGENESIS IN THE LUNG” Lung Cancer. 34:S17-25; Travis, W. D. et al. (1991) “NEUROENDOCRINE TUMORS OF THE LUNG WITH PROPOSED CRITERIA FOR LARGE-CELL NEUROENDOCRINE CARCINOMA. AN ULTRASTRUCTURAL, IMMUNOHISTOCHEMICAL, AND FLOW CYTOMETRIC STUDY OF 35 CASES” Am J Surg Pathol 15:529-53; Brambilla, E. et al. (2001) “THE NEW WORLD HEALTH ORGANIZATION CLASSIFICATION OF LUNG TUMOURS” Eur Respir J. 18:1059-68).

Such microscopic evaluations of tissue samples is complex and difficult. Moreover, no “gold-standard” exists for defining neuroendocrine differentiation (Carnaghi, C. et al. (2001) “CLINICAL SIGNIFICANCE OF NEUROENDOCRINE PHENOTYPE IN NON-SMALL-CELL LUNG CANCER” Ann Oncol. 12:S119-23). The absence of an effective diagnostic standard complicates the management and treatment of neuroendocrine tumors (Obërg, K. (2001) “CHEMOTHERAPY AND BIOTHERAPY IN THE TREATMENT OF NEUROENDOCRINE TUMOURS,” Ann Oncol 12:S111-4).

Researchers have attempted to apply the principles of molecular biology in order to identify molecular markers that would facilitate the diagnosis of neuroendocrine tumor types (see, for example, Japanese Patent Document JP 58,198,758A2; and U.S. Pat. Nos. 5,766,888; 5,856,097; 5,866,323; 5,965,362; 5,976,790; 5,985,240; 5,998,154; 6,132,724; 6,166,176; 6,180,082; 6,225,049; 6,238,877; 6,251,586; 6,335,167; and 6,358,491). Certain proteins, such as chromogranin A (CgA) and neuron-specific enolase (NSE) have been identified as having specific potential use in the clinical diagnosis of neuroendocrine tumors (Seregni, E. et al. (2000) “LABORATORY TESTS FOR NEUROENDOCRINE TUMOURS” Q J Nucl Med. 44:2241). Non-SCLC neuroendocrine tumors have been reported to overexpress CgA whereas SCLC tumors exhibit elevated NSE levels. Id. Lui, W.-O. et al. (2001) “HIGH LEVEL AMPLIFICATION OF 1P32-33 AND 2p22-24 IN SMALL CELL LUNG CARCINOMAS” Intl. J Oncol. 19:451-457 used comparative genomic hybridization analysis to identify chromosomal abnormalities in SCLC tumor cells. Through such analysis, several genetic regions were found to be amplified (i.e., 1p32, 2p23, 1p32, and 2p32). A loss of heterozygosity (LOH) is observed on 3p, 13q and 17p in nearly all SCLC tumors (Yokota et al. (1987) “LOSS OF HETEROZYGOSITY ON CHROMOSOMES 3, 13 AND 17 IN SMALL CELL CARCINOMA AND ON CHROMOSOME 3 IN ADENOCARCINOMA OF THE LUNG” Proc. Natl. Acad. Sci. (U.S.A.) 84:9252-9256. Similarly, deletions in 11q have been correlated with the presence of AC and TC tumors (Walch, A. K. et al. (1998) “TYPICAL AND ATYPICAL CARCINOID TUMORS OF THE LUNG ARE CHARACTERIZED BY 11Q DELETIONS AS DETECTED BY COMPARATIVE GENOMIC HYBRIDIZATION” Am J Pathol. 153:1089-98).

While such efforts have succeeded in identifying quantitative differences in mutations affecting various genes (for example finding that p53 is inactivated in >90% of SCLC tumors, but in only >50% of non-SCLC tumors, or that p16 abnormalities arise in <1% of SCLC tumors but in ˜66% of non-SCLC tumors), clear correlations that would support a definitive differential diagnosis of tumor type has not been fully achieved (see, Ignacio, I. et al. (2001) “MOLECULAR GENETICS OF SMALL CELL LUNG CARCINOMA” Semin Oncol. 28:3-13; Carnaghi, C. et al. (2001) “CLINICAL SIGNIFICANCE OF NEUROENDOCRINE PHENOTYPE IN NON-SMALL-CELL LUNG CANCER” Ann Oncol. 12:S 119-23). In this regard, one recent study found no statistically significant correlation between any individual marker and response to chemotherapy for non-SCLC tumors (Gajra, A. et al. (2002) “THE PREDICTIVE VALUE OF NEUROENDOCRINE MARKERS AND P53 FOR RESPONSE TO CHEMOTHERAPY AND SURVIVAL IN PATIENTS WITH ADVANCED NON-SMALL CELL LUNG CANCER” Lung Cancer. 36:159-65). Thus, a need remains for a usable molecular marker approach that could distinguish between the different types of neuroendocrine tumors.

cDNA microarrays have been employed to analyze gene expression patterns in human cancers (DeRisi, J. et al. (1996) “USE OF A cDNA MICROARRAY TO ANALYSE GENE EXPRESSION PATTERNS IN HUMAN CANCER” Nature Genetics 14:457-60). Such efforts have combined DNA amplification technologies (such as T7-based RNA amplification) with cDNA microarrays in order to improve the discriminating power of the analysis (Luo, L. et al. (1999) “GENE EXPRESSION PROFILES OF LASER-CAPTURED ADJACENT NEURONAL SUBTYPES” Nature Medicine 5:117-22; Bonner, R. F. et al. (1997) “LASER CAPTURE MICRODISSECTION: MOLECULAR ANALYSIS OF TISSUE” Science 278:1481,1483; Schena, M. et al. (1995) “QUANTITATIVE MONITORING OF GENE EXPRESSION PATTERNS WITH A COMPLEMENTARY DNA MICROARRAY” Science 270:467-70).

Despite all such progress, no fully successful method for distinguishing between the neuroendocrine tumor types, and of thus accurately diagnosing neuroendocrine cancers has yet been achieved. The present invention is, in part, directed to such needs.

### SUMMARY OF THE INVENTION

This invention relates to methods and compositions for the diagnosis of neuroendocrine lung cancers. The present invention permits one to accurately classify pulmonary neuroendocrine tumors based on their genome-wide expression profile without further manipulation. A hierarchical clustering of all genes classifies these tumors according to World Health Organization (WHO) histological type. The selection of genes with significant variance resulted in the identification of 198 transcripts, many of which have potentially important biological and clinical implications. The present invention thus defines and provides groups of genes that identify each tumor type, and permits one to derive a molecular signature that distinguishes each subtype of neuroendocrine tumor.

In detail, the invention provides a method for determining whether a candidate cell is a neuroendocrine tumor cell, wherein the method comprises the steps of:
(A) determining the profile of expression of a plurality of genes of the candidate cell; and
(B) comparing such determined profile of expression with the profile of expression of the genes of a small cell lung cancer cell, a large cell neuroendocrine carcinoma cell, a typical carcinoid tumor cell or an atypical carcinoid tumor cell;
   to thereby determine whether the candidate cell is a neuroendocrine tumor cell.

The invention particularly concerns the embodiment of such method wherein the method additionally permits a determination of neuroendocrine tumor cell type. The invention further concerns the embodiments of such methods wherein the method determines whether the candidate cell is a small cell lung cancer (SCLC) neuroendocrine tumor cell, a large cell neuroendocrine carcinoma (LCNEC) neuroendocrine tumor cell, a typical carcinoid (TC) neuroendocrine tumor cell, or an atypical carcinoid (AC) neuroendocrine tumor cell.

The invention particularly concerns the embodiments of such methods wherein the plurality of genes includes one or more genes selected from the group consisting of C5, CPE, GRIA2, RIMS2, ORC4L, CSF2RB, GGH, NPAT, NR3C1, P311, PRKAA2, PTK6, APRT, ARF4L, ARHGDIA, ARL7, ATP6F, CDC20, CDC34, CLDN11, COMT, CSTF1, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCR1, SIP2-28, SIVA, SURF 1, TADA3L, TK1, TYMSTR, and VATI, and especially wherein the plurality of genes includes one or more genes selected from the group consisting of GGH and CPE.

The invention further concerns the embodiments of such methods wherein step (A) of the methods comprise incubating RNA of the candidate cell, or DNA or RNA amplified from such RNA, in the presence of a plurality of genes, or fragments or RNA transcripts thereof, under conditions sufficient to cause RNA to hybridize to complementary DNA or RNA molecules; and detecting hybridization that occurs.

The invention additionally concerns the embodiments of such methods wherein the plurality of genes, or polynucleotide fragments or RNA transcripts thereof, are distinguishably arrayed in a microarray. The invention particularly concerns the embodiments of such methods wherein the microarray comprises arrayed genes, or polynucleotide fragments or RNA transcripts thereof, that are differentially expressed in neuroendocrine tumor cells relative to normal cells.

The invention particularly concerns the embodiments of such methods wherein the microarray comprises arrayed genes, or polynucleotide fragments or RNA transcripts thereof, that are differentially expressed in small cell lung cancer (SCLC) neuroendocrine tumor cells relative to large cell neuroendocrine carcinoma (LCNEC) neuroendocrine tumor cells.

The invention particularly concerns the embodiments of such methods wherein the microarray comprises arrayed genes, or polynucleotide fragments or RNA transcripts thereof, that are differentially expressed in small cell lung cancer (SCLC) neuroendocrine tumor cells relative to typical carcinoid (TC) neuroendocrine tumor cells.

The invention particularly concerns the embodiments of such methods wherein the microarray comprises arrayed genes, or polynucleotide fragments or RNA transcripts thereof, that are differentially expressed in small cell lung cancer (SCLC) neuroendocrine tumor cells relative to atypical carcinoid (AC) neuroendocrine tumor cells.

The invention particularly concerns the embodiments of such methods wherein the microarray comprises arrayed genes, or polynucleotide fragments or RNA transcripts thereof, that are differentially expressed in large cell neuroendocrine carcinoma (LCNEC) neuroendocrine tumor cells relative to atypical carcinoid (AC) neuroendocrine tumor cells.

The invention particularly concerns the embodiments of such methods wherein the microarray comprises arrayed genes, or polynucleotide fragments or RNA transcripts thereof, that are differentially expressed in large cell neuroendocrine carcinoma (LCNEC) neuroendocrine tumor cells relative to typical carcinoid (TC) neuroendocrine tumor cells.

The invention particularly concerns the embodiments of such methods wherein the arrayed genes, or polynucleotide fragments or RNA transcripts thereof, include one or more genes selected from the group consisting of C5, CPE, GRLA2, RIMS2, ORC4L, CSF2RB, GGH, NPAT, NR3C1, P311, PRKAA2, PTK6, APRT, ARF4L, ARHGDIA, ARL7, ATP6F, CDC20, CDC34, CLDN11, COMT, CSTF1, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCR1, SIP2-28, SIVA, SURF1, TADA3L, TK1, TYMSTR, and VATI.,

The invention especially concerns the embodiments of such methods wherein the arrayed genes, or polynucleotide fragments or RNA transcripts thereof, include one or more genes selected from the group consisting of GGH and CPE, or polynucleotide fragments or RNA transcripts thereof.

The invention particularly concerns the embodiments of such methods wherein the microarray comprises arrayed genes, or polynucleotide fragments or RNA transcripts thereof, that are differentially expressed in large cell neuroendocrine carcinoma (LCNEC) neuroendocrine tumor cells relative to atypical carcinoid (AC) neuroendocrine tumor cells.

The invention particularly concerns the embodiments of such methods wherein the microarray comprises arrayed genes, or polynucleotide fragments or RNA transcripts thereof, that are differentially expressed in typical carcinoid (TC) neuroendocrine tumor cells relative to atypical carcinoid (AC) neuroendocrine tumor cells The invention additionally concerns a microarray of genes, or polynucleotide fragments or RNA transcripts thereof for distinguishing a neuroendocrine tumor cell, the microarray comprising a solid support having greater than 10 genes, or polynucleotide fragments or RNA transcripts thereof, distinguishably arrayed in spaced apart regions, wherein the microarray comprises a sufficient number of genes, or polynucleotide fragments or RNA transcripts thereof, that are differentially expressed in a small cell lung cancer (SCLC) cell, a large cell neuroendocrine carcinoma (LCNEC) neuroendocrine tumor cell, a typical carcinoid (TC) neuroendocrine tumor cell, or an atypical carcinoid (AC) neuroendocrine tumor cell, relative to a normal cell or a cell belonging to a different neuroendocrine tumor cell type, to permit the microarray to distinguish a pulmonary neuroendocrine tumor cell.

The invention particularly concerns the embodiment of such microarray wherein the microarray comprises a sufficient number of genes, or polynucleotide fragments or RNA transcripts thereof, that are differentially expressed in a neuroendocrine tumor cell relative to a normal cell to permit the microarray to distinguish between a neuroendocrine tumor cell and a normal cell.

The invention particularly concerns the embodiments of such microarrays wherein the microarray comprises a sufficient number of genes, or polynucleotide fragments or RNA transcripts thereof, that are differentially expressed in a small cell lung cancer (SCLC) neuroendocrine tumor cell relative to a large cell neuroendocrine carcinoma (LCNEC) neuroendocrine tumor cell to permit the microarray to distinguish between a small cell lung cancer (SCLC) neuroendocrine tumor cell and a large cell neuroendocrine carcinoma (LCNEC) neuroendocrine tumor cell.

The invention particularly concerns the embodiments of such microarrays wherein the microarray comprises a sufficient number of genes, or polynucleotide fragments or RNA transcripts thereof, that are differentially expressed in a small cell lung cancer (SCLC) neuroendocrine tumor cell relative to a typical carcinoid (TC) neuroendocrine tumor cell to permit the microarray to distinguish between a small cell lung cancer (SCLC) neuroendocrine tumor cell and a typical carcinoid (TC) neuroendocrine tumor cell.

The invention particularly concerns the embodiments of such microarrays wherein the microarray comprises a sufficient number of genes, or polynucleotide fragments or RNA transcripts thereof, that are differentially expressed in a small cell lung cancer (SCLC) neuroendocrine tumor cell relative to an atypical carcinoid (AC) neuroendocrine tumor cell to permit the microarray to distinguish between a small cell lung cancer (SCLC) neuroendocrine tumor cell and an atypical carcinoid (AC) neuroendocrine tumor cell.

The invention particularly concerns the embodiments of such microarrays wherein the microarray comprises a sufficient number of genes, or polynucleotide fragments or RNA transcripts thereof, that are differentially expressed in a large cell neuroendocrine carcinoma (LCNEC) neuroendocrine tumor cell relative to a typical carcinoid (TC) neuroendocrine tumor cell to permit the microarray to distinguish between a large cell neuroendocrine carcinoma (LCNEC) neuroendocrine tumor cell and a typical carcinoid (TC) neuroendocrine tumor cell.

The invention particularly concerns the embodiments of such microarrays wherein the microarray comprises a sufficient number of genes, or polynucleotide fragments or RNA transcripts thereof, that are differentially expressed in a large cell neuroendocrine carcinoma (LCNEC) neuroendocrine tumor cell relative to an atypical carcinoid (AC) neuroendocrine tumor cell to permit the microarray to distinguish between a large cell neuroendocrine carcinoma (LCNEC) neuroendocrine tumor cell and an atypical carcinoid (AC) neuroendocrine tumor cell.

The invention particularly concerns the embodiments of such microarrays wherein the microarray comprises a sufficient number of genes, or polynucleotide fragments or RNA transcripts thereof, that are differentially expressed in a typical carcinoid (TC) neuroendocrine tumor cell relative to an atypical carcinoid (AC) neuroendocrine tumor cell to permit the microarray to distinguish between a typical carcinoid (TC) neuroendocrine tumor cell and an atypical carcinoid (AC) neuroendocrine tumor cell.

The invention particularly concerns the embodiments of such microarrays wherein the genes or polynucleotide fragments or RNA transcripts thereof of the microarray include one or more genes selected from the group consisting of C5, CPE, GRIA2, RIMS2, ORC4L, CSF2RB, GGH, NPAT, NR3C1, P311, PRKAA2, PTK6, APRT, ARF4L, ARHGDIA, ARL7, ATP6F, CDC20, CDC34, CLDN11, COMT, CSTF1, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCR1, SIP2-28, SIVA, SURF1, TADA3L, TK1, TYMSTR, and VATI, or a polynucleotide fragment or RNA transcript thereof.

The invention further concerns the embodiments of such microarrays wherein the genes or polynucleotide fragments or RNA transcripts thereof of the microarray include one or more genes selected from the group consisting of GGH and CPE, or a polynucleotide fragment or RNA transcript thereof.

### BRIEF DESCRIPTION OF THE FIGURES

FIG. 1 shows the hierarchical clustering of genes with statistically significant variance (p<0.004) among all tumor samples.

FIG. 2 shows the hierarchical clustering of 198 genes, created by enforcing the classification of 17 tumors.

FIGS. 3A and 3B show the expression of genes of large cell neuroendocrine tumor cells and typical carcinoid tumor cells.

FIG. 4 shows a dendrogram of pulmonary NE tumors based on expression of 198 genes. Seventeen cases of the NE tumors were sorted by one-way hierarchical clustering based on the expression similarities of 198 genes that were selected from 9,984 genes based on the expression changes in the three subtype tumors with significant statistical difference (F-test, p<0.004). Medium gray, light gray, and black signal indicate that expression of these genes is higher, lower or equal to the median level of expression in all samples, respectively. White represents missing genes or poor quality data. TC: typical carcinoid; SC: small cell lung cancer; LC: large cell neuroendocrine carcinoma; SC+LC: a tumor sample with 90% SC and 10% LC. The numbers are the case numbers of the tumor samples.

FIGS. 5A, 5B, 5C, 5D, 5E, and 5F show comparisons of expression changes detected by microarrays and real-time quantitative RT-PCR. RNA isolated from LCM cells was examined in triplicates for expression of three representative genes upregulated in each tumor subtype. The gene expression changes detected by real-time RT-PCR (FIG. 5A-C) were presented here in comparisons with those derived from cDNA microarray analysis (FIG. 5D-F). The expression of each gene in the RT-PCR analysis was normalized first by expression of the 18S ribosomal gene in the same cell line and then by the expression of that gene in the BEAS-2B control cells. CPE: carboxypeptidase E; P311: a gene of neuronal marker; CDC20: human homolog gene for S. cerevisiae cell division cycle 20 gene. TC: typical carcinoid; SC: small cell lung cancer; LC: large cell neuroendocrine carcinoma. The 17 pulmonary NET cases were arranged from left to right in each panel in the same order of 1240, 1672, 11169, 11934, 12454, 12878, 890, 1047, 11061, 12346, 12457, 12893, 13369, 10110, 10249, 10373, and 12700. The primer pairs for RT-PCR are: CPE: (SEQ ID NO:2) 5′-TTGTCCGAGACCTTCAAGGTAAC-3′ and (SEQ ED NO:3) 5′-CCTTTGCGGATGTAACATCGT-3′; P311: (SEQ ID NO:4) 5′-TGGGTCAGTCAAGAACCATTTC-3′ and (SEQ ID NO:5) 5′-ACTTCCTTTGGGACAGGAAGTCT-3′; and CDC20: (SEQ ID NO:6) 5′-CTGAACGGTTTTGATGTAGAGGAA-3′ and (SEQ ID NO:7) 5′-CCCTCTGGCGCATTTTGT-3′.

FIGS. 6A and 6B show the results of Kaplan-Meier Survival rates of 54 cases of pulmonary NET patients as function of CPE or GGH expression. FIG. 6A shows the survival rates of patients with positive and negative CPE stains on pulmonary NET cells. The survival rate (76%) for the patients with the positive CPE are statistically significant (p=0.023) higher than that (27%) with the negative stain. FIG. 6B shows the inverse correlation of the survival rates to the GGH expression in pulmonary NET cells. The survival rates to positive and negative GGH stains in pulmonary NET cells were 28% and 83%, respectively, with the statistic significance (p=0.0035). X indicates censored samples.

### DESCRIPTION OF THE PREFERRED EMBODIMENTS

The invention concerns methods and compositions for the diagnosis of neuroendocrine lung cancers. Lung cancer is a leading cause of cancer-related deaths (Franceschi, S. et al. (1999) “THE EPIDEMIOLOGY OF LUNG CANCER,” Ann. Oncol. 10 Suppl 5:S3-6). Pulmonary neuroendocrine tumors (NETs) account for 20-30% of lung cancer cases and lung cancer is the leading cause of cancer-related death (Parkin, D. M. et al. (1999) “GLOBAL CANCER STATSTICS,” CA Cancer J Clin 49:33-64, 1). The observed continuous relative increase in the incidence of SCLC (Junker, K. et al. (2000) “PATHOLOGY OF SMALL-CELL LUNG CANCER,” J. Cancer Res. Clin. Oncol. 126:361-368) reflects cigarette smoking, lack of effective methods for early diagnosis and inadequate predictive markers of aggressive lung cancer types.

Pulmonary NETs include low-grade typical carcinoid (TC), intermediate-grade atypical carcinoid (AC), and high-grade large cell neuroendocrine carcinoma (LCNEC) and small cell lung cancer (SCLC) (Travis, W. D. et al. (1998) “REPRODUCIBILITY OF NEUROENDOCRINE LUNG TUMOR CLASSIFICAON,” Hum Pathol. 29:272-279). TC, AC and LCNEC collectively comprise only 3%-5% of all pulmonary malignancies, whereas SCLC accounts for 15%-25% (Travis, W. D. et al. (1998) “REPRODUCIBILITY OF NEUROENDOCRINE LUNG TUMOR CLASSIFICATION,” Hum Pathol. 29:272-279; Travis, W. D. et al. (1991)”, “NEUROENDOCRINE TUMORS OF THE LUNG WITH PROPOSED CRITERIA FOR LARGE-CELL NEUROENDOCRINE CARCINOMA. AN ULTRASTRUCTURAL, IMMUNOHISTOCHEMICAL, AND FLOW CYTOMETRIC STUDY OF 35 CASES,” Am J Surg Pathol. 15:529-553). The prognostic relevance of pulmonary NETs has changed significantly since the recent recognition of the LCNEC subtype (Travis, W. D. et al. (1998) “REPRODUCIBILITY OF NEUROENDOCRINE LUNG TUMOR CLASSIFICATION,” Hum Pathol. 29:272-279; Travis, W. D. et al. (1991) ”, “NEUROENDOCRINE TUMORS OF THE LUNG WITH PROPOSED CRITERIA FOR LARGE-CELL NEUROENDOCRINE CARCINOMA. AN ULTRASTRUCTURAL, IMMUNOHISTOCHEMICAL, AND FLOW CYTOMETRIC STUDY OF 35 CASES,” Am J Surg Pathol. 15:529-553). The 5- and 10-year survival rates for TC are 87% and 87%, for AC are 56% and 35%, for LCNEC are 27% and 9%, and for SCLC are 9% and 5%, respectively. Pulmonary NETs have a similar morphologic appearance with organoid, trabecular or rosette-like pattern, and the immunohistochemical staining for neuroendocrine markers: chromogranin, synaptophysin, and neural cell adhesion molecule (NCAM, CD56). To distinguish these tumors from non-small cell lung cancers (NSCLC), some cases are analyzed by electron microscopy for the presence of neuroendocrine granules. Prior to the present invention, no specific molecular markers had been identified that could distinguish subtypes of pulmonary NETs and, other than clinical stage at presentation, the tumor mitotic index is the only independent histologic predictor of survival. The current treatment for patients with TC and AC is surgical resection, because these tumors grow slowly and are frequently detected as solitary pulmonary lesions. In contrast, surgical resection is feasible in less than one third of the LCNEC patients, with or without neoadjuvant treatment. Unfortunately, at the time of diagnosis, most SCLC are disseminated and prognosis is poor. Thus, accurate diagnosis of the pulmonary NET subtypes is essential for appropriate treatment and prediction of clinical outcome (Travis, W. D. et al. (1998) “SURVIVAL ANALYSIS OF 200 PULMONARY NEUROENDOCRINE TUMORS WITH CLARIFICATION OF CRITERIA FOR ATYPICAL CARCINOID AND ITS SEPARATION FROM TYPICAL CARCINOID,” Am J Surg Pathol. 22:934944; Zacharias, J. et al. (2003) “LARGE CELL NEUROENDOCRINE CARCINOMA AND LARGE CELL CARCINOMAS WITH NEUROENDOCRINE MORPHOLOGY OF THE LUNG: PROGNOSIS AFTER COMPLETE RESECTION AND SYSTEMATIC NODAL DISSECTION,” Ann. Thorac. Surg. 75:348-352).

Neuroendocrine tumors are a distinct subset of lung cancers that share morphologic, ultrastructural, immunohistochemical, and molecular characteristics. As indicated above, the term neuroendocrine tumors encompasses small cell lung cancer (SCLC) tumors, large cell neuroendocrine carcinomas, typical carcinoid (TC) tumors and atypical carcinoid (AC) tumors. All neuroendocrine tumors have similar morphologic appearance with organoid, trabecular or rosettelike pattern; immunohistochemical staining for chromogranin (Cga), synaptophysin, neuron-specific enolase (NSE), neural cell adhesion molecule (NCAM), and the presence of neuroendocrine granules, which can be detected by electron microscopy (Fisher, E. R. et al. (1978) “COMPARATIVE HISTOPATHOLOGIC, HISTOCHEMICAL, ELECTRON MICROSCOPIC AND TISSUE CULTURE STUDIES OF BRONCHIAL CARCINOIDS AND OAT CELL CARCINOMAS OF THE LUNG,” Am J Clin Pathol 69: 165-172).

The dramatic differences in survival exhibited by the different neuroendocrine malignancies reflect fundamental differences in biological behavior and therapeutic approaches in these tumors (Travis, W. D., et al. (1998) “SURVIVAL ANALYSIS OF 200 PULMONARY NEUROENDOCRINE TUMORS: WITH CLARIFICATION OF CRITERIA FOR ATYPICAL CARCINOID AND ITS SEPARATION FROM TYPICAL CARCINOID,” Am J Surg Pathol 22:934-944). Current treatment for patients with TC involves surgical resection because the tumors are slow growing and frequently detected as solitary pulmonary lesions. In less than one third of patients with LCNEC, surgical resection is possible without neoadjuvant treatment. Unfortunately, at the time of diagnosis, most SCLC tumors are disseminated, treatment is not effective and the prognosis is poor. Thus, accurate diagnosis of each type of pulmonary neuroendocrine tumors is essential for successful clinical outcome.

The combined use of light microscopy, immunohistochemistry and electron microscopy has increased the oncologist's ability to differentiate different subtypes of neuroendocrine tumors and has provided clues regarding their pathogenesis. However, little information is available on genetic changes associated with each type of neuroendocrine tumors.

Over the past decade, there have been significant changes in the classification of pulmonary neuroendocrine tumors in order to improve prediction of their biological behavior. The accurate diagnosis of each pulmonary tumor subtype is critical for decisions of therapy. A diagnosis based on light microscopic examination, specifically in differentiation of SCLC from other pulmonary NETs is often challenging. Unfortunately, there are no molecular markers to aid in differentiation of each tumor subtype.

In accordance with the methods of the present invention, the analysis of genome-wide gene expression in neuroendocrine tumors from cDNA microarray data (often referred to as “unsupervised learning”) accurately distinguishes each tumor type. The pattern of gene expression has been found to correlate with each subtype assigned by light microscopy according to WHO/LASLSC classification (Histopathological classification of these tumors is based on the 1999 WHO Classification (Travis, W. D. et al. (1999) “HISTOLOGIC TYPING OF LUNG AND PLEURAL TUMORS” (Ed 3). Berlin, Germany, Springer).

Microarray technology is widely used to identify changes in gene expression accompanying altered cell physiology during development, cell cycle progression, drug treatment or disease progression. Related phenotypes are usually accompanied by related patterns of cellular transcripts that can be used to characterize these changes. The present invention exploits the recent development of DNA microarray technology (see, for example, DeRisi, J. et al. (1996) “USE OF A cDNA MICROARRAY TO ANALYSE GENE EXPRESSION PATTERNS IN HUMAN CANCER” Nature Genetics 14:457-60; Luo, L. et al. (1999) “GENE EXPRESSION PROFILES OF LASER-CAPTURED ADJACENT NEURONAL SUBTYPES” Nature Medicine 5:117-22; Bonner, R. F. et al. (1997) “LASER CAPTURE MICRODISSECTION: MOLECULAR ANALYSIS OF TISSUE” Science 278:1481,1483; Schena, M. et al. (1995) “QUANTITATIVE MONITORING OF GENE EXPRESSION PATTERNS WITH A COMPLEMENTARY DNA MICROARRAY” Science 270:467-70) to analyze genome-wide changes that may distinguish these tumors and discover molecular markers. The identification of such markers and their subsequent use ion the diagnosis and monitoring of neuroendocrine cancers permits a more effective selection of treatment modalities for individual patients.

The analysis of changes in gene expression in clinical specimens is complicated by the mixture of tumor and normal cells, as well as stromal, vascular, and other cells obtained in biopsy. In addition, the heterogeneity of cell type hinders the study of gene expression profiles in cancer cells. Although the principles of the present invention may be used with tissue biopsies and other tissue samples, most preferably, the analysis will be conducted with single cells. Such single cells can be isolated using any of a variety of methods, however, the use of laser capture microdissection (LCM) is preferred. Laser capture microdissection is a procedure that permits the harvesting of a specific cell population directly from frozen sections. The procedure involves fixing the desired cells to a thermoplastic film following infrared laser pulse to avoid “contamination” by other cell populations (Emmert-Buck, M. R. et al. (1996) “Laser Capture Microdissection,” Science 274:998-1001; Goldsworthy, S. M. et al. (1999) “EFFECTS OF FIXATION ON RNA EXTRACTION AND AMPLIFICATION FROM LASER CAPTURE MICRODISSECTED TISSUE,” Molecular Carcinogenesis, 1999, 86-91; Luo, L. et al. (1999) “GENE EXPRESSION PROFILES OF LASER-CAPTURED ADJACENT NEURONAL SUBTYPES” Nature Medicine 5:117-22).

Most preferably, the PixCell™ LCM system (Arcturus, Moutain View, Calif.) is used for laser capture microdissection (Bonner, R. F., et al. (1997) “LASER CAPTURE MICRODISSECTION: MOLECULAR ANALYSIS OF TISSUE,” Science 278:1481, 1483). The examples described below illustrate the desirability of isolating tumor cells from vascular and inflammatory components frequently found in surgical specimens of lung cancer and other vascular tumors.

The present invention thus permits one to distinguish between different neuroendocrine tumor subtypes based on their expression profiles. Preferably, such analysis will involve a comparison of the expression of multiple genes, and is accomplished by assessing the extent or presence of hybridization occurring between RNA transcripts (or cDNA copies thereof) of a candidate cell and genes, or polynucleotide fragments or RNA transcripts thereof of a reference cell that are differentially expressed in some or all neuroendocrine tumor cells. As used herein, a gene is said to be “differentially expressed” in a tumor cell if detection of its expression facilitates the determination that a candidate cell is or is not a tumor cell. As used herein, the term “polynucleotide fragment” refers to a polynucleotide that is either a portion of a gene, cDNA or RNA molecule, or a complement of such molecules, and which possesses a length of at least 10 nucleotide residues, at least 15 nucleotide residues, at least 20 nucleotide residues, at least 25 nucleotide residues, at least 35 nucleotide residues, at least 50 nucleotide residues, at least 75 nucleotide residues, at least at least 100 nucleotide residues, at least 150 nucleotide residues, or at least 200 nucleotide residues.

Clones containing suitable genes, and from which suitable polynucleotide fragments or RNA transcripts can be made, are obtainable from Incyte Genomics (www.incyte.com). The present invention provides a preferred set of 198 genes that are particularly suited for use in such analysis. Clones of these genes are commercially available from Incyte Genomics using the Incyte Clone ID No. information provided in Table 2. Preferably the analysis will be conducted using 10%, 20%, 50%, 70%, 80%, 90% or all of these 198 genes, alone or in combination with other genes, or polynucleotide fragments or RNA transcripts thereof. These 198 genes have been found to define three different cluster groups. The analysis may involve a comparison of the expression of genes belonging to the same cluster group, or to two or more different cluster groups.

cDNA microarrays are preferably performed on a solid surface, such as a chip or slide. Preferably, such surfaces will contain multiple human genes, or polynucleotide fragments or RNA transcripts thereof, distinguishably arrayed. As used herein, the term “distinguishably arrayed” is intended to denote that such gene's (or its fragment or transcript)'s location on the surface is distinct or distinguishable from the locations of other gene(s) that may be bound to the support.

Most preferably, the array will contain gene fragments of hundreds or thousands of human genes. A glass slide containing gene fragments of 9,984 human genes (provided by the Advanced Technology Center of the National Cancer Institute) is preferably employed. Clones and arrays are also available from Incyte Genomics, Palo Alto, Calif., and other sources.

For analyzing such microarrays, nucleic acid, most preferably RNA, is isolated from candidate neuroendocrine cells. Any of a wide variety of amplification procedures may be employed. In a preferred embodiment of the invention, a T7-based RNA amplification procedure in employed, such as that described by Luo, L. et al. (1999) (“GENE EXPRESSION PROFILES OF LASER-CAPTURED ADJACENT NEURONAL SUBTYPES” Nature Medicine 5:117-22). To facilitate the analysis, the amplified material is preferably labeled, as with a radioactive, fluorescent, chemiluminescent, enzymatic, haptenic, or other label, and incubated with the arrayed gene fragments under conditions suitable for nucleic acid hybridization to occur (see, for example, Schena, M. et al. (1995) “QUANTITATIVE MONITORING OF GENE EXPRESSION PATTERNS WITH A COMPLEMENTARY DNA MICROARRAY” Science 270:467-70).

The hybridized array are then analyzed for their pattern of hybridization. Detection of hybridization, e.g., detection of the labeled amplified material hybridized to a region of the array, indicates that the gene present at such region was expressed by the candidate cell being analyzed. Most preferably, such analysis will employ an automated scanning device, such as a GenePix 4000A Laser Scanner (Axon Instruments, Inc., Foster City, Calif.) in conjunction with software for conducting such analysis. The BRB ArrayTools (ver 2.0) is preferred for this purpose (http://linus.nci.nih.gov/BRB-ArrayTools.html).

Having now generally described the invention, the same will be more readily understood through reference to the following examples, which are provided by way of illustration, and are not intended to be limiting of the present invention, unless specified.

### EXAMPLE 1

### cDNA Microarray

In order to identify molecular markers of pulmonary neuroendocrine tumors, the gene expression profile of clinical samples from patients with TC, LCNEC, and SCLC is analyzed by cDNA microarrays, preferably as follows:

Tissue Collection And RNA Quality Assessment. Archived, frozen lung tumor tissues are collected from hospitals over an 11 year period. Tumor tissues are flash-frozen at surgery and stored at −80° C. until used. The frozen tumor tissue block is prepared with O.C.T. mount medium and the quality of total RNA of each sample is evaluated by spectrophotometery and gel electrophoresis after phenol/chloroform extraction from one frozen section. Histopathological classification of these tumors is based on the 1999 WHO Classification (Travis, W. D. et al. (1999) “HISTOLOGIC TYPING OF LUNG AND PLEURAL TUMORS” (Ed 3). Berlin, Germany, Springer). Two large cell neuroendocrine carcinomas (case 1240 and 1672) are confirmed by demonstrating the neuorendocrine immuno-phenotype with positive NCAM (CD56) staining. Table 1 summarizes clinical findings in the pulmonary NE tumors.

TABLE 1
Clinical Features Of 17 Patients With Pulmonary Neuroendocrine Tumors

| | | Sex | | Age | | |
|---|---|---|---|---|---|---|
| Histology | | Male | Female | Range | Mean | Smoking |
| TC | (n = 11) | 7 | 4 | 35-68 | 50 | 7 (64%) |
| LCNEC | (n = 2) | 2 | 0 | 59-60 | 60 | 2 (100%) |
| SCLC | (n = 4) | 3 | 1 | 43-75 | 65 | 4 (100%) |
| TOTAL | (n = 17) | 12 | 5 | 35-75 | 65 | 13 (100%) |

Laser Capture Microdissection Of 17 Neuroendocrine Tumors. Frozen tumor tissue (0.5×0.5×0.5 cm) are embedded in O.C.T. in a cryomold, and immersed immediately in dry ice-cold 2-methylbutane at −50° C. Sections of frozen tissue (8 mm) are mounted on silane coated glass slides and kept at −80° C. until use. The slides are immediately fixed by immersion in 70% ethanol, stained with H&E and air-dried for 10 minutes after xylene treatment.

The PixCelI™ LCM system (Arcturus, Moutain View, Calif.) is used for LCM (Bonner, R. F., et al. (1997) “LASER CAPTURE MICRODISSECTION: MOLECULAR ANALYSIS OF TISSUE,” Science 278: 1481,1483). Tumor cells are fused to transfer film by thermal adhesion after laser pulse and were then transferred into tubes containing solution D in the Strategene Micro RNA isolation kit that contains gaunidinium thiocyanate (GTC) and beta-mercaptoethanol. For each specimen, 15 to 18 frozen sections are used to maximize the quantity of RNA. Total RNA is extracted using a Micro RNA isolation kit (Strategene, La Jolla, Calif.) according to the manufacturer's instructions. Purified total RNA was resuspended in 11 ml of diethyl pyrocarbonate (DEPC), treated water, and used directly for RNA amplification and subjected to cDNA microarray analysis (Schena, M. et al. (1995) “QUANTITATIVE MONITORING OF GENE EXPRESSION PATTERNS WITH A COMPLEMENTARY DNA MICROARRAY,” Science 270(5235):467-70; DeRisi, J. et al. (1996) “USE OF A cDNA MICRO ARRAY TO ANALYSE GENE EXPRESSION PATTERNS IN HUMAN CANCER,” Nature Genetics 14:457-60, Lyer, R. P. et al (1999) “MODIFIED OLIGONUCLEOTIDES—SYNTHESIS, PROPERTIES AND APPLICATIONS,” Curr. Opin. Mol. Ther. 1:344-358).

RNA Amplification. The RNA amplification procedure used is preferably as described by Luo, L. et al. (1999) (“GENE EXPRESSION PROFILES OF LASER-CAPTURED ADJACENT NEURONAL SUBTYPES,” Nature Med 5: 117-122). The method relies on attaching a T7 promoter sequence to the oligo(dT) primer. A preferred such sequence for synthesis of the first strand cDNA is SEQ ID NO.:1:

5′ TCTAGTCGAC GGCCAGTGAA TTGTAATACG ACTCACTATA
GGGCGTTTTT TTTTTTTTTT TTTTTT 3′

After second strand cDNA synthesis, amplified RNA is generated using 17 RNA polymerase and the double-stranded cDNA molecules as targets for the linear amplification. The T7 promoter sequence is regenerated in subsequent rounds by priming the first strand cDNA synthesis reaction with random hexamers (Amersham Biosciences, Piscataway, NJ). The quality and quantity of amplified RNA were evaluated spectrophotometricaly and by migration in 2.4% agarose gel electrophoresis, respectively.

Cell Culture. BEAS-2B cell line (Amstad, P. et al. (1988) “NEOPLASTIC TRANSFORMATION OF A HUMAN BRONCHIAL EPITHELIAL CELL LINE BY A RECOMBINANT RETROVIRUS ENCODING VIRAL HARVEY RAS,” Mol Carcinog. 1988 1:151-60) is cultured in a serum-free medium, LHC-9 (Biofluids, Rockville, Md.). Total RNA is isolated from cells with Trizol, followed by phenol/chloroform and isopropanol extraction and purification (Stratagene, La Jolla, Calif.). Two rounds of amplified RNA are generated from the cell line as described above.

Microarrays Hybridization. cDNA microarrays are performed in duplicate for each sample on glass slides containing 9,984 human genes which were provided by the Advanced Technology Center of the National Cancer Institute. BEAS-2B amplified RNA (8 μg) is labeled with Cy5-dUTP and test samples (4 mg each) are labeled with Cy3-dUTP using Superscript 11 (Invitrogen, Carlsbad, Calif.). Briefly, RNA is incubated with Cy3-dUTP (or Cy5-dUTP) (Perkin Elmer Life Sciences, Boston, Mass.) at 42° C. for lh to synthesize the first strand of cDNA. The reaction is stopped by addition of 5 μl 0.5M EDTA and 10 μl 1N NaOH followed by incubation at 65° C. for 60 min. After neutralization, the samples are purified by centrifugation with a Microcon 30 (Millipore Corp., Bedford, Mass.) to remove unincorporated nucleotides and salts. The Cy3- and Cy5-labeled samples of each pair are combined and heated at 100° C. for 2 min. After centrifugation for 10 minutes, the samples are placed onto the center of a glass microarray slide and hybridized at 65° C. for 16h. The slides are washed to a final stringency of 0.2×SSC at room temperature for 2 min prior to analysis.

Image And Statistic Analysis. Hybridized array slides are scanned with a GenePix 4000A Laser Scanner (Axon Instruments, Inc., Foster City, Calif.). Analysis is performed using BRB ArrayTools (ver 2.0) developed by Drs. Richard Simon and Amy Peng (http://linus.nci.nih.gov/BRB-ArrayTools.html). Hierarchical clustering was performed on 8,987 clones with log-ratios present in at least 4 samples for each gene.

### EXAMPLE 2

### cDNA Microarray Results

The results of the microarray analysis are obtained using Laser Capture Microdissection (LCM) as follows:

Laser Capture Microdissection (LCM) Of Clinical Samples. Use of LCM improves the sample preparation of microarray analysis by avoiding contamination with other cell types. (Emmert-Buck, M. R. et al. (1996) “Laser Capture Microdissection,” Science 274:998-1001). This selection is particularly desirable for analysis of tumors from lung, prostate, overy, and others (Ornstein, D. K. et al. (2000) “PROTEOMIC ANALYSIS OF LASER CAPTURE MICRODISSECTED HUMAN PROSTATE CANCER AND IN VITRO PROSTATE CELL LINES,” Electrophoresis 21(11):2235-2242; Mirura, K. et al. (2002) “LASER CAPTURE MICRODISSECTION AND MICROARRAY EXPRESSION ANALYSIS OF LUNG ADENOCARCINOMA REVEALS TOBACCO SMOKING—AND PROGNOSIS RELATED MOLECULAR PROFILES,” Cancer Res. 62:3244-3250; Ono, K. et al. (2000) “IDENTIFICATION BY cDNA MICROARRAY OF GENES INVOLVED IN OVARIAN CARCINOGENESIS,” Cancer Res. 60:5007-5011). Tumor cells are selected by LCM from frozen sections. High quality RNA is obtained from these dissected materials. .

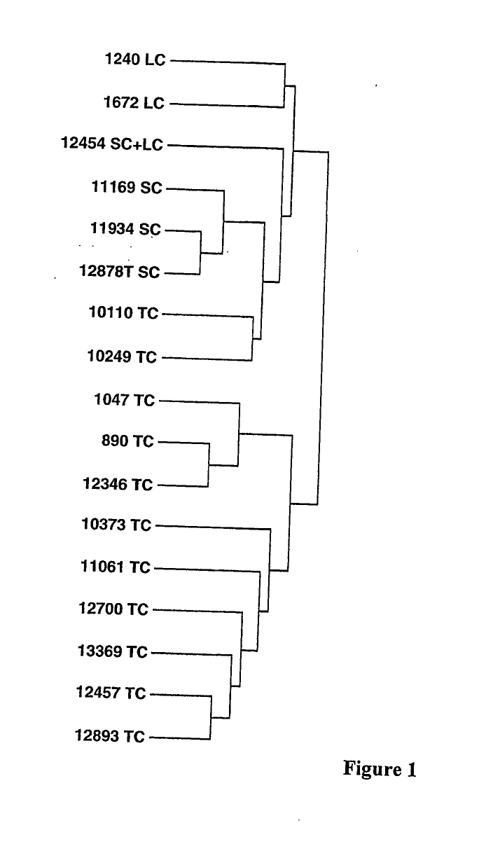

Microarray Analysis Of Gene Expression Profiles Of Pulmonary Neuroendocrine Tumors. The invention tested the hypothesis that gene expression profiling using cDNA microarray analysis can effectively identify subtypes of pulmonary neuroendocrine tumors classified by light microscopy according to WHO recommendations. Hierarchical clustering of 8,987 human genes, often referred to as unsupervised learning, separated samples into clusters based on overall similarity in gene expression without prior knowledge of sample identity. The hierarchical clustering of genes with statistically significant variance (p<0.004) among all tumor samples is displayed in FIG. 1. After decoding the specimens, it was immediately apparent that clustering based on genome-wide expression divides the tumors into their assigned WHO classification with 100% accuracy. Tumor samples from TC, LCNEC and SCLC clusters with their respective subtype indicating similarities of gene expression shared by these tumors. The length of the branches indicates the relatedness of neuroendocrine tumors. Three distinct groups of tumors can be identified by this display. The sample, which contains features of LCNEC and SCLC clusters between LCNEC and SCLC with a shorter distance to SCLC. Thus, the data support the molecular classification that predicted morphological classification of human pulmonary neuroendocrine tumors. The data indicates that WHO proposed morphological classification of pulmonary neuroendocrine tumors correspond to a significant similarity of their molecular profiles.

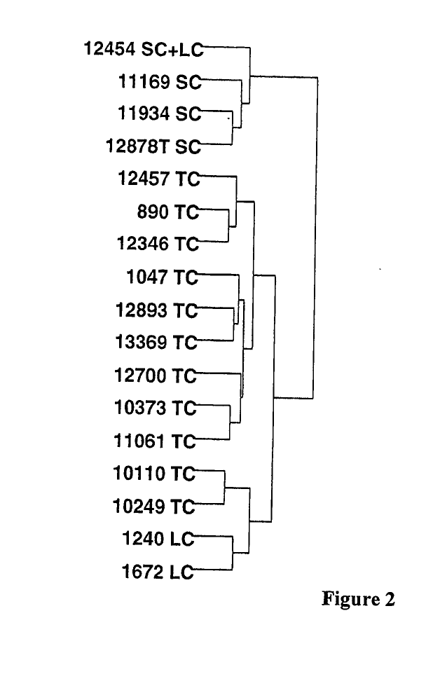

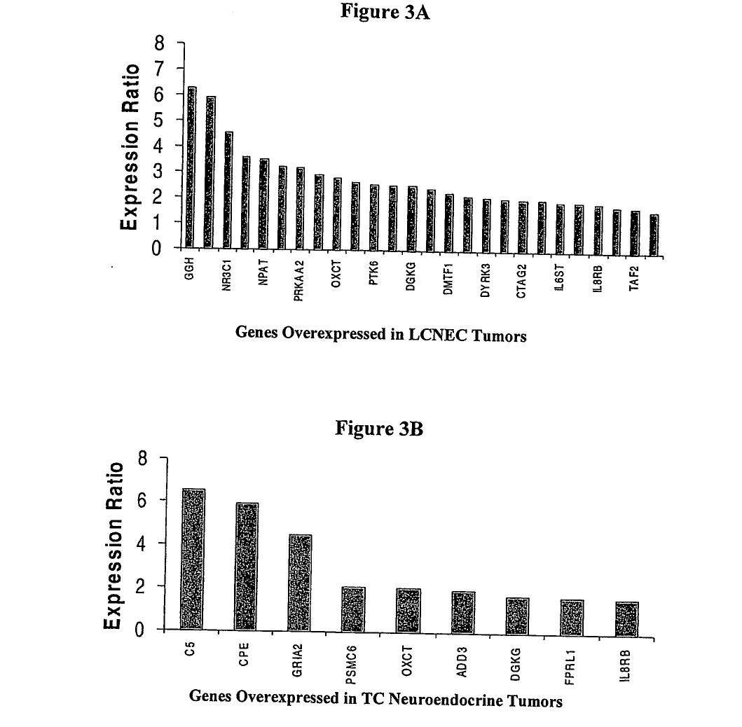

The Class Comparison Tool is used to select genes differentially expressed among each tumor type at an assigned statistical significance level. The F-test, which measures levels of variance in gene expression among each sample, is used to compare the defined classes of tumors using BRB ArrayTool. This analysis results in the identification of a set of 198 genes that have statistically significant variance (p<0.004, Table 2). Having selected these 198 genes, another hierarchical clustering can be created by enforcing the classification of 17 tumors (FIG. 2). The results show that the tumors cluster together in 3 groups in complete agreement with the pre-assigned morphological classification. Samples from LCNEC cluster closer to TC than to SCLC and the tumor that contained features of small and large neuroendocrine cells clustered with SCLC which confirms the molecular relatedness identified by genome-wide expression in clinical behavior of these tumors. The results show that most of the 198 selected genes could be assigned to major functional groups that have been previously implicated in cancer development (Table 3). In particular, decreased expression of genes that oppose cell survival pathway, such as BCL2 antagonist-killer, BAK1, and caspase 4, are common in all 3 types of neuroendocrine tumors, whereas TC and LCNEC have an additional >2.5-fold decrease in expression of BAS and TNF receptor-interacting kinase, RIPK1. These features indicate that these tumors lack opposing effects on BCL2, as contrasted to overexpression of BCL2, which leads to survival advantage in certain types of lymphomas (Cleary, M. L. et al. (1986) “CLONING AND STRUCTURAL ANALYSIS OF cDNAS FOR BCL-2 AND A HYBRID BCL-2/IMMUNOGLOBULIN TRANSCRIPT RESULTING FROM THE T(14; 18) TRANSLOCATION,” Cell. 47(1): 19-28) (FIG. 3).

TABLE 2
Genes Having Statistically Significant Variance in Expression in Neuroendocrine Tumor
Cells

| | | | | |
|---|---|---|---|---|
| Unique | | Gene Symbol | Incyte | UG |
| ID No. | Description | (Map) | Clone ID No. | Cluster |

Cluster #1

| | | | | |
|---|---|---|---|---|
| 166807 | glutamate receptor, lonotropic, | GRIA2 | IncytePD: 1505977 | Hs.89582 |
| | AMPA 2 | [4q32-q33] | | |
| | Neuronal Marker, TM Receptor | | | |
| 159877 | carboxypeptidase E | CPE | IncytePD: 2153373 | Hs.75360 |
| | Secreted Lys Neuronal M | [4q32.3] | | |
| 161598 | origin recognition complex, subunit | ORC4L | IncytePD: 2728840 | Hs.55055 |
| | 4 (yeast homolog)-like | [2q22-q23] | | |
| 167158 | complement component 5 | C5 | IncytePD: 1712663 | Hs.1281 |
| | Infl. Resp. VP. Extracellular | [9q32-q34] | | |

Cluster #2

| | | | | |
|---|---|---|---|---|
| 167153 | gamma-glutamyl hydrolase | GGH | IncytePD: 1997967 | Hs.78619 |
| | (conjugase, | [8q12.1] | | |
| | folylpolygammaglutamyl hydrolase) | | | |
| | Protease, Lys | | | |
| 160605 | P311 protein | P311 | IncytePD: 1555545 | Hs.142827 |
| | Invasion marker, Adhesion | [5q21.3] | | |
| | Plaques | | | |
| 169429 | nuclear receptor subfamily 3, | NR3C1 | IncytePD: 629077 | Hs.75772 |
| | group C, member 1 | [5q31] | | |
| | Glucocort. Rec/TF | | | |
| 165192 | synaptojanin2 | SYNJ2 | IncytePD: 3954785 | Hs.61289 |
| | IP3 5-Phosphatase | [6q25-26] | | |
| 165784 | adducin 3 (gamma) | ADD3 | IncytePD: 1481225 | Hs.324470 |
| | Cytoschel | [10q24.2-q24.3] | | |
| 163031 | KIAA0751 gene product | KIAA0751 | IncytePD: 2369544 | Hs.153610 |
| | | [8q23.1] | | |
| 166328 | proteasome (prosome, macropain) | PSMC6 | IncytePD: 1488021 | Hs.79357 |
| | 26S subunit, ATPase, 6 | [12q15] | | |
| | Proteasome | | | |
| 168061 | formyltetrahydrofolate | FTHFD | IncytePD: 2104145 | Hs.9520 |
| | dehydrogenase | [3q21.3] | | |
| | NADPH Sx, Folic Acid One-carbon | | | |
| | meth | | | |
| 168141 | diacylglycerol kinase, gamma | DGKG | IncytePD: 2568547 | Hs.89462 |
| | (90 kD) | [3q27-q28] | | |
| 165076 | PI-3-kinase-related kinase SMG-1 | SMG1 | IncytePD: 4253663 | Hs.110613 |
| | RNA Survellance | [16p12.3] | | |
| 167103 | TAF2 RNA polymerase II, TATA | TAF2 | IncytePD: 998069 | Hs.122752 |
| | box binding protein (TBP)- | [8q24.12] | | |
| | associated factor, 150 kD | | | |
| | TATA Box TF | | | |
| 169391 | eukaryotic translation initiation | EIF2S1 | IncytePD: 1224219 | Hs.151777 |
| | factor 2, subunit 1 (alpha, 35 kD) | [14q23.3] | | |
| | polysome | | | |
| 166789 | zinc finger protein 202 | ZNF202 | IncytePD: 1997937 | Hs.9443 |
| | Transcriptional Repressor | [11q23.3] | | |
| 167316 | solute carrier family 24 | SLC24A1 | IncytePD: 2200079 | Hs.173092 |
| | (sodium/potassium/calcium | [15q22] | | |
| | exchanger), member 1 | | | |
| | Sodium/potassium/calcium | | | |
| | exchanger | | | |
| 168700 | formyl peptide receptor-like 1 | FPRL1 | IncytePD: 523635 | Hs.99855 |
| | Integram | [19q13.3-13.4] | | |
| | Membr/Migration/Expressed in | | | |
| | Lung | | | |
| 165576 | interleukin 6 signal transducer | IL6ST | IncytePD: 2172334 | Hs.82065 |
| | (gp130, oncostatin M receptor) | [5q11] | | |
| 168276 | integrin, beta-like 1 (with EGF-like | ITGBL1 | IncytePD: 1258790 | Hs.82582 |
| | repeat domains) | [13q33] | | |
| 169180 | interleukin 8 receptor, beta | IL8RB | IncytePD: 561992 | Hs.846 |
| | | [2q35] | | |
| 160957 | protein kinase, AMP-activated, | PRKAA2 | IncytePD: 2507648 | Hs.2329 |
| | alpha 2 catalytic subunit | [1p31] | | |
| 160617 | colony stimulating factor 2 | CSF2RB | IncytePD: 1561352 | Hs.285401 |
| | receptor, beta, low-affinity | [22q13.1] | | |
| | (granulocyte-macrophage) | | | |
| 160429 | PTK6 protein tyrosine kinase 6 | PTK6 | IncytePD: 3255437 | Hs.51133 |
| | Non-Receptor, Sensitizes to EGF | [20q13.3] | | |
| 160237 | nuclear protein, ataxia- | NPAT | IncytePD: 2308525 | Hs.89385 |
| | telanglectasia locus | [11q22-q23] | | |
| | Osteogenesis Imperfecta | | | |
| 167125 | tumor necrosis factor receptor | TNFRSF6 | IncytePD: 2205246 | Hs.82359 |
| | superfamily, member 6 | [10q24.1] | | |
| 164652 | platelet-derived growth factor | PDGFRB | IncytePD: 1821971 | Hs.76144 |
| | receptor, beta polypeptide | [5q31-q32] | | |
| 161117 | ATP-binding cassette, sub-family | ABCG2 | IncytePD: 1501080 | Hs.194720 |
| | G (WHITE), member 2 | [4q22] | | |
| | Multidrug Resistance | | | |
| 161896 | collagen, type XV, alpha 1 | COL15A1 | IncytePD: 4287342 | Hs.83164 |
| | | [9q21-q22] | | |
| 159813 | protein tyrosine phosphatase, non- | PTPN12 | IncytePD: 1382374 | Hs.62 |
| | receptor type 12 | [7q11.23] | | |
| | PEST Dom; p-c-Abl, Ctx. Cell | | | |
| | shape/motility | | | |
| 164573 | cyclin D binding Myb-like | DMTF1 | lncytePD: 1637517 | Hs.5671 |
| | transcription factor 1 | [7q21] | | |
| | Not reported to be Expressed in | | | |
| | Lung | | | |
| 169384 | solute carrier family 22 (organic | SLC22A1LS | IncytePD: 3842669 | Hs.300076 |
| | cation transporter), member 1-like | [11p15.5] | | |
| | antisense | | | |
| | Organic-Cation Transporter-Like 2- | | | |
| | Antisense | | | |
| 165393 | ESTs, Weakly similar to 2109260A | | IncytePD: 3202075 | Hs.351699 |
| | B cell growth factor [H. sapiens] | | | |
| 168169 | 3-oxoacid CoA transferase | OXCT | IncytePD: 1685342 | Hs.177584 |
| | mitochondrial matrix coenzyme A | [5p13] | | |
| | from succinyl-CoA to acetoacetate | | | |
| 165617 | prolactin receptor | PRLR | IncytePD: 3427560 | Hs.1906 |
| | | [5p14-p13] | | |
| 169432 | interleukin 13 receptor, alpha 2 | IL13RA2 | IncytePD: 3360476 | Hs.25954 |
| | | [Xq13.1-q28] | | |
| 166812 | myelin protein zero-like 1 | MPZL1 | IncytePD: 2057323 | Hs.287832 |
| | extracellular membrane face | [1q23.2] | | |
| 168428 | runt-related transcription factor 3 | RUNX3 | IncytePD: 885297 | Hs.170019 |
| | | [1p36] | | |
| 167180 | S100 calcium-binding protein A4 | S100A4 | IncytePD: 1222317 | Hs.81256 |
| | (calcium protein, calvasculin, | [1q21] | | |
| | metastasin, murine placental | | | |
| | homolog) | | | |
| | cell cycle progression, Associated | | | |
| | with mets | | | |
| 161533 | cleavage stimulation factor, 3′ pre- | CSTF2 | IncytePD: 4016254 | Hs.693 |
| | RNA, subunit 2, 64 kD | [Xq21.33] | | |
| | RNA processing/modification | | | |
| 165588 | small nuclear RNA activating | SNAPC4 | IncytePD: 2224902 | Hs.113265 |
| | complex, polypeptide 4, 190 kD | [9q34.3] | | |
| 164799 | epithelial membrane protein 3 | EMP3 | IncytePD: 780992 | Hs.9999 |
| | cell-cell interactions. Promotes | [19q13.3] | | |
| | Apoptosis | | | |
| 161709 | hypothetical protein FLJ11560 | FLJ11560 | IncytePD: 1990361 | Hs.301696 |
| | | [9p12] | | |
| 164868 | guanylate binding protein 2, | GBP2 | IncytePD: 1610993 | Hs.171862 |
| | interferon-inducible | [1pter-p13.2] | | |
| | GTP-ase | | | |
| 160233 | dual-specificity tyrosine-(Y)- | DYRK3 | IncytePD: 614679 | Hs.38018 |
| | phosphorylation regulated kinase 3 | [1q32] | | |
| | Cell growth, P-histones, | | | |
| | Transcription | | | |
| 165400 | hypothetical brain protein my040 | MY040 | IncytePD: 2048144 | Hs.124854 |
| | Overexp Lung neuroendocrine | [7q35-q36] | | |
| | tumors | | | |
| 165957 | pancreatic lipase-related protein 2 | PNLIPRP2 | IncytePD: 885032 | Hs.143113 |
| | Hydrolyse | [10q26.12] | | |
| 160054 | GTP-binding protein homologous | SEC4L | IncytePD: 1824556 | Hs.302498 |
| | to Saccharomyces cerevisiae | [17q25.3] | | |
| | SEC4 | | | |
| | Sec vesicles SC | | | |
| 162475 | cancer/testis antigen 2 | CTAG2 | IncytePD: 849425 | Hs.87225 |
| | melanomas, non-small-cell lung | [Xq28] | | |
| | carcinomas, bladder, Prostate, H/N | | | |
| 169182 | testis-specific ankyrin motif | LOC56311 | IncytePD: 2013272 | Hs.73073 |
| | containing protein | [7q31] | | |
| 162912 | nectin 3 | DKFZP566B084 | IncytePD: 2680168 | Hs.21201 |
| | PVRL1; may be a membrane | [3q13] | | |
| | glycoprotein | | | |
| 163475 | hypothetical protein | FLJ20485 | IncytePD: 2299818 | Hs.98806 |
| | 7q22.1 102-113 | [7q22.1] | | |
| 164927 | heterogeneous nuclear | HNRPA0 | IncytePD: 637639 | Hs.77492 |
| | ribonucleoprotein A0 | [5q31] | | |
| | RNA processing/modification | | | |
| 160630 | homeo box D9 | HOXD9 | IncytePD: 2956581 | Hs.236646 |
| | RNA processing/modification | [2q31-q37] | | |
| 160367 | v-jun avian sarcoma virus 17 | JUN | IncytePD: 1969563 | Hs.78465 |
| | oncogene homolog | [1p32-p31] | | |
| | Associated with transl in Tumors | | | |
| 163762 | ESTs | [17] | IncytePD: 1743234 | Hs.120854 |
| 162247 | very large G protein-coupled | VLGR1 | IncytePD: 942207 | Hs.153692 |
| | receptor 1 | [5q13] | | |
| | transports Ca2+ during excitation- | | | |
| | contraction | | | |
| 167219 | pumilio (Drosophila) homolog 1 | PUM1 | IncytePD: 3333130 | Hs.153834 |
| | | [1p35.2] | | |

Cluster #3

| | | | | |
|---|---|---|---|---|
| 165171 | keratin 18 | KRT18 | IncytePD: 1435374 | Hs.65114 |
| | | [12q13] | | |
| 165052 | CDC20 (cell division cycle 20, S. cerevisiae, | CDC20 | IncytePD: 2469592 | Hs.82906 |
| | homolog) | [9q13-q21] | | |
| | Cell cycle, microtubule-dependent | | | |
| | processes | | | |
| 167948 | pim-1 oncogene | PIM1 | IncytePD: 2679117 | Hs.81170 |
| | S.T kinase Hematop Cells | [6p21.2] | | |
| 161954 | ATPase, H+ transporting, | ATP6F | IncytePD: 5017148 | Hs.7476 |
| | lysosomal (vacuolar proton pump) | [1p32.3] | | |
| | 21 kD | | | |
| | Vacuolar H Transporter | | | |
| 162391 | polymerase (DNA directed), | POLE3 | IncytePD: 961082 | Hs.108112 |
| | epsilon 3 (p17 subunit) | [9q33] | | |
| | DNA Replication | | | |
| 166635 | keratin 5 (epidermolysis bullosa | KRT5 | IncytePD: 3432534 | Hs.195850 |
| | simplex, Dowling- | [12q12-q13] | | |
| | Meara/Kobner/weber-Cockayne | | | |
| | types) | | | |
| 160035 | flap structure-specific | FEN1 | IncytePD: 2050085 | Hs.4756 |
| | endonuclease 1 | [11q12] | | |
| | DNA Repair/UV rad protection | | | |
| 161774 | calcium and integrin binding | SIP2-28 | IncytePD: 4626895 | Hs.10803 |
| | protein (DNA-dependent protein | [15q25.3-q26] | | |
| | kinase interacting protein) | | | |
| 162207 | membrane protein of cholinergic | VATI | IncytePD: 2060308 | Hs.157236 |
| | synaptic vesicles | [17q21] | | |
| | vesicular transport | | | |
| 161163 | guanylate kinase 1 | GUK1 | IncytePD: 2506427 | Hs.3764 |
| | Sx GTP/GMP | [1q32-q41] | | |
| 161223 | CD27-binding (Siva) protein | SIVA | IncytePD: 2356635 | Hs.112058 |
| | tumor necrosis receptor (TFNR) | [22] | | |
| | superfamily | | | |
| 161211 | capping protein (actin filament), | CAPG | IncytePD: 2508570 | Hs.82422 |
| | gelsolin-like | [2cen-q24] | | |
| 161948 | claudin 11 (oligodendrocyte | CLDN11 | IncytePD: 4144001 | Hs.31595 |
| | transmembrane protein) | [3q26.2-q26.3] | | |
| 161391 | interleukin 17F | IL17F | IncytePD: 1610083 | Hs.272295 |
| | | [6p12] | | |
| 162571 | phosphofructokinase, liver | PFKL | IncytePD: 885601 | Hs.155455 |
| | | [21q22.3] | | |
| 164504 | cathepsin C | CTSC | IncytePD: 1822716 | Hs.10029 |
| | Lys Prot Degr | [11q14.1-q14.3] | | |
| 160565 | aminoacylase 1 | ACY1 | IncytePD: 1812955 | Hs.334707 |
| | L-aa Sx salvage path | [3p21.1] | | |
| 169551 | glycogen synthase kinase 3 beta | GSK3B | IncytePD: 2057908 | Hs.78802 |
| | target of Akt, llk1, Reg jun, myb, | [3q13.3] | | |
| | etc. | | | |
| 166914 | methyltransferase-like 1 | METTL1 | IncytePD: 1603584 | Hs.42957 |
| | S-adenosylmethionine-binding mo | [12q13] | | |
| 167738 | cytochrome P450, subfamily | CYP27B1 | IncytePD: 1749727 | Hs.199270 |
| | XXVIIB (25-hydroxyvitamin D-1- | [12q13.1-q13.3] | | |
| | alpha-hydroxylase), polypeptide 1 | | | |
| | drug metabolism and synthesis of | | | |
| | cholesterol, steroids | | | |
| 160938 | GrpE-like protein cochaperone | HMGE | IncytePD: 2074154 | Hs.151903 |
| | cooperates with mitochondrial | [4p16] | | |
| | hsp70 I | | | |
| 162734 | wingless-type MMTV integration | WNT7A | IncytePD: 2622566 | Hs.72290 |
| | site family, member 7A | [3p25] | | |
| | Regulates Steroid responses | | | |
| 165813 | caspase 4, apoptosis-related | CASP4 | IncytePD: 2304121 | Hs.74122 |
| | cysteine protease | [11q22.2-q22.3] | | |
| 159898 | pituitary tumor-transforming 1 | PTTG1 | IncytePD: 1748705 | Hs.252587 |
| | | [5q35.1] | | |
| 161244 | ADP-ribosylation factor 4-like | ARF4L | IncytePD: 2852403 | Hs.183153 |
| | GTP-binding proteins. ARF4L is c | [17q12-q21] | | |
| 160715 | cell division cycle 34 | CDC34 | IncytePD: 1857493 | Hs.76932 |
| | | [19p13.3] | | |
| 163787 | pyrroline-5-carboxylate reductase 1 | PYCR1 | IncytePD: 1702266 | Hs.79217 |
| | Proline Sx | [17q24] | | |
| 160127 | phosphoglycerate mutase 1 (brain) | PGAM1 | IncytePD: 3032691 | Hs.181013 |
| | | [10q25.3] | | |
| 160323 | 5-aminoimidazole-4-carboxamide | ATIC | IncytePD: 2056149 | Hs.90280 |
| | ribonucleotide | [2q35] | | |
| | formyltransferase/IMP | | | |
| | cyclohydrolase | | | |
| | Purine BioSx | | | |
| 164850 | interleukin-1 receptor-associated | IRAK1 | IncytePD: 1872067 | Hs.182018 |
| | kinase 1 | [Xq28] | | |
| 165583 | 7-dehydrocholesterol reductase | DHCR7 | IncytePD: 3518380 | Hs.11806 |
| | | [11q13.2-q13.5] | | |
| 165039 | thymidine kinase 1, soluble | TK1 | IncytePD: 2055926 | Hs.105097 |
| | two forms have been identified in | [17q23.2-q25.3] | | |
| | animal cells | | | |
| 167964 | cyclin-dependent kinase inhibitor | CDKN2A | IncytePD: 2740235 | Hs.1174 |
| | 2A (melanoma, p16, inhibits | [9p21 | | |
| | CDK4) | | | |
| 167223 | guanine nucleotide binding protein | GNB1 | IncytePD: 3562795 | Hs.215595 |
| | (G protein), beta polypeptide 1 | [1p36.21-36.33] | | |
| | Ras GTPase, Contains 7 wd | | | |
| | repeats | | | |
| 167931 | cleavage stimulation factor, 3′ pre- | CSTF1 | IncytePD: 1635008 | Hs.172865 |
| | RNA, subunit 1, 50 kD | [20q13.2] | | |
| | RNA, transducin-like repeats | | | |
| 163690 | hexabrachion (tenascin C, | HXB | IncytePD: 1453450 | Hs.289114 |
| | cytotactin) | [9q33] | | |
| 161955 | contactin 2 (axonal) | CNTN2 | IncytePD: 4014715 | Hs.2998 |
| | | [1q32.1] | | |
| 160275 | structure specific recognition | SSRP1 | IncytePD: 2055773 | Hs.79162 |
| | protein 1 | [11q12 | | |
| 168110 | TAF12 RNA polymerase II, TATA | TAF12 | IncytePD: 1297269 | Hs.82037 |
| | box binding protein (TBP)- | [1p35.1] | | |
| | associated factor, 20 kD | | | |
| 160102 | protein disulfide isomerase related | ERP70 | IncytePD: 1824957 | Hs.93659 |
| | protein (calcium-binding protein, | [10] | | |
| | intestinal-related) | | | |
| | Sevretion; ER | | | |
| 167116 | nucleoside phosphorylase | NP | IncytePD: 2453436 | Hs.75514 |
| | adenosine deaminase (ADA) | [14q13.1] | | |
| | serves a key role in purine | | | |
| | catabolism; Def = SCID | | | |
| 160802 | prohibitin | PHB | IncytePD: 1625169 | Hs.75323 |
| | Tumor suppressor, Blocks DNA | [17q21] | | |
| | Sx; Breast CA | | | |
| 161643 | ADP-ribosylation factor-like 7 | ARL7 | IncytePD: 3115514 | Hs.111554 |
| | GTP-binding protein | [2q37.2] | | |
| 162343 | LIM domain kinase 2 | LIMK2 | IncytePD: 958513 | Hs.278027 |
| | Rho-induced reorganization of the | [22q12.2] | | |
| | actin cytoskeleton | | | |
| 162727 | protein tyrosine kinase 9-like (A6- | PTK9L | IncytePD: 3999291 | Hs.6780 |
| | related protein) | [3p21.1] | | |
| 160262 | DEAD/H (Asp-Glu-Ala-Asp/His) | DDX28 | IncytePD: 2663948 | Hs.155049 |
| | box polypeptide 28 | [16q22.1] | | |
| | probable atp-binding rna helicase | | | |
| 165790 | surfeit 1 | SURF1 | IncytePD: 1921567 | Hs.3196 |
| | Mit. Resp Enz | [9q33-q34] | | |
| 168638 | histone deacetylase 7A | HDAC7A | IncytePD: 1968721 | Hs.275438 |
| | | [12q13.1] | | |
| 168079 | epithelial membrane protein 1 | EMP1 | IncytePD: 1624024 | Hs.79368 |
| | cell-cell interactions. Promotes | [12p12.3] | | |
| | Apoptosis | | | |
| 160999 | Rho-specific guanine nucleotide | P114-RHO-GEF | IncytePD: 1734113 | Hs.6150 |
| | exchange factor p114 | [19p13.3] | | |
| | cell growth and motility; Dbl, PH | | | |
| | dom | | | |
| 161790 | KIAA0469 gene product | KIAA0469 | IncytePD: 2674277 | Hs.7764 |
| | | [1p36.23] | | |
| 169691 | ubiquitin carrier protein | E2-EPF | IncytePD: 2057823 | Hs.174070 |
| | E2 enzyme activity | [17p12-p11] | | |
| 163682 | diptheria toxin resistance protein | DPH2L2 | IncytePD: 1810821 | Hs.324830 |
| | required for diphthamide | [1p34] | | |
| | biosynthesis (Saccharomyces)-like 2 | | | |
| 168266 | proteasome (prosome, macropain) | PSME3 | IncytePD: 1308112 | Hs.152978 |
| | activator subunit 3 (PA28 gamma; | [17q12-q21] | | |
| | Ki) | | | |
| 161374 | polymerase (DNA-directed), alpha | POLA2 | IncytePD: 3179113 | Hs.81942 |
| | (70 kD) | [11q13.1] | | |
| | RNA Processing | | | |
| 164646 | galactose-4-epimerase, UDP- | GALE | IncytePD: 1807294 | Hs.76057 |
| | Rate-lim for Sx glycoproteins and | [1p36-p35] | | |
| | glycolipids | | | |
| 162150 | apolipoprotein L | APOL1 | IncytePD: 2056987 | Hs.114309 |
| | | [22q13.1] | | |
| 164206 | type I transmembrane protein Fn14 | FN14 | IncytePD: 1402615 | Hs.10086 |
| | similar to murine Fgfrp2 | [16p13.3] | | |
| 162623 | BCL2-antagonist/killer 1 | BAK1 | IncytePD: 2055687 | Hs.93213 |
| | | [6p21.3] | | |
| 162244 | Rho GDP dissociation inhibitor | ARHGDIA | IncytePD: 2055640 | Hs.159161 |
| | (GDI) alpha | [17q25.3] | | |
| 164586 | inosine triphosphatase (nucleoside | ITPA | IncytePD: 1931265 | Hs.6817 |
| | triphosphate pyrophosphatase) | [20p] | | |
| | Ins Phos phosphatase | | | |
| 165483 | PDGFA associated protein 1 | PDAP1 | IncytePD: 3032825 | Hs.278426 |
| | Enhances PDGFA | [7q22.1] | | |
| 166195 | adenine phosphoribosyltransferase | APRT | IncytePD: 2751387 | Hs.28914 |
| | Sx AMP purine/pyrimidine Met | [16q24] | | |
| 166960 | Apg12 (autophagy 12, S. cerevisiae)- | APG12L | IncytePD: 2058537 | Hs.264482 |
| | like | [5q21-q22] | | |
| 167505 | thiosulfate sulfurtransferase | TST | IncytePD: 1988239 | Hs.351863 |
| | (rhodanese) | [22q13.1] | | |
| | Mitoch detox cyanide | | | |
| 168642 | suppression of tumorigenicity 14 | ST14 | IncytePD: 478960 | Hs.56937 |
| | (colon carcinoma, matriptase, | [11q24-q25] | | |
| | epithin) | | | |
| | Protease ECM | | | |
| 167170 | GS2 gene | DXS1283E | IncytePD: 1567995 | Hs.264 |
| | | [Xp22.3] | | |
| 161754 | actin, gamma 2, smooth muscle, | ACTG2 | IncytePD: 3381870 | Hs.78045 |
| | enteric | [2p13.1] | | |
| 166010 | receptor (TNFRSF)-interacting | RIPK1 | IncytePD: 2180031 | Hs.296327 |
| | serine-threonine kinase 1 | [6p25.3] | | |
| 161794 | secretory carrier membrane protein 2 | SCAMP2 | IncytePD: 3123858 | Hs.238030 |
| | Vesic Traff, Secretpry path | [15q23-q25] | | |
| 167591 | catechol-O-methyltransferase | COMT | IncytePD: 605019 | Hs.240013 |
| | Sx dopamine, epinephrine, and | [22q11.21] | | |
| | norepinephrine | | | |
| 162587 | polymerase (RNA) II (DNA | POLR2D | IncytePD: 696002 | Hs.194638 |
| | directed) polypeptide D | [2q21] | | |
| | RNA Processing | | | |
| 169071 | capping protein (actin filament) | CAPZB | IncytePD: 1853163 | Hs.333417 |
| | muscle Z-line, beta | [1p36.1] | | |
| 160467 | polymerase (DNA directed), delta | POLD2 | IncytePD: 2056172 | Hs.74598 |
| | 2, regulatory subunit (50 kD) | [7p13] | | |
| | RNA Processing | | | |
| 162176 | C2f protein | C2F | IncytePD: 5096975 | Hs.12045 |
| | | [12p13] | | |
| 167706 | GDP-mannose pyrophosphorylase B | GMPPB | IncytePD: 1486983 | Hs.28077 |
| | N-linked oligosaccharides | [3p21.31] | | |
| 160803 | phenylalanine-tRNA synthetase- | FARSL | IncytePD: 1808260 | Hs.23111 |
| | like | [19p13.2] | | |
| | Reg. in tumors and cell cycle | | | |
| 169254 | polymerase (DNA directed), mu | POLM | IncytePD: 771715 | Hs.46964 |
| | RNA Processing | [7p13] | | |
| 167351 | myosin-binding protein H | MYBPH | IncytePD: 3010959 | Hs.927 |
| | | [1q32.1] | | |
| 163276 | ESTs, Weakly similar to I37356 | [7] | IncytePD: 2383065 | Hs.25892 |
| | epithelial microtubule-associated | | | |
| | protein, 115K [H. sapiens] | | | |
| 167135 | excision repair cross- | ERCC1 | IncytePD: 2054529 | Hs.59544 |
| | complementing rodent repair | [19q13.2-q13.3] | | |
| | deficiency, complementation group | | | |
| | 1 (includes overlapping antisense | | | |
| | sequence) | | | |
| 160478 | G5b protein | G5B | IncytePD: 1942845 | Hs.73527 |
| | | [6p21.3] | | |
| 162631 | transcriptional adaptor 3 (ADA3, | TADA3L | IncytePD: 3990209 | Hs.158196 |
| | yeast homolog)-like (PCAF histone | [3p25.2] | | |
| | acetylase complex) | | | |
| | PCAF histone acetilase complex | | | |
| 163921 | glucosamine-6-phosphate | GNPI | IncytePD: 1653911 | Hs.278500 |
| | isomerase | [5q21] | | |
| | Hydrolase | | | |
| 160098 | mitochondrial ribosomal protein | MRPL49 | IncytePD: 1755793 | Hs.75859 |
| | L49 | [11q13] | | |
| 161058 | multiple endocrine neoplasia I | MEN1 | IncytePD: 1693847 | Hs.24297 |
| | | [11q13] | | |
| 160038 | BCL2-antagonist of cell death | BAD | IncytePD: 3967780 | Hs.76366 |
| | | [11q13.1] | | |
| 162220 | FK506-binding protein 1A (12 kD) | FKBP1A | IncytePD: 4059193 | Hs.349972 |
| | Interacts with TGF beta | [20p13] | | |
| 161026 | Xq28, 2000 bp sequence contg. | HSXQ28ORF | IncytePD: 1669254 | Hs.6487 |
| | ORF | [Xq28] | | |
| | 3′ eDNA Repair xonuclease activity | | | |
| 167607 | heat shock protein 75 | TRAP1 | IncytePD: 1960722 | Hs.182366 |
| | HSP90 fam, Binds to TNFR | [16p13.3] | | |
| 167713 | likely ortholog of maternal | KIAA0175 | IncytePD: 3805046 | Hs.184339 |
| | embryonic leucine zipper kinase | [9p11.2] | | |
| | regulation of fatty acid synthesis | | | |
| 165648 | dual specificity phosphatase 4 | DUSP4 | IncytePD: 740878 | Hs.2359 |
| | negatively regulate MAPK. Anti- | [8p12-p11] | | |
| | oncogene | | | |
| 161574 | frequently rearranged in advanced | FRAT2 | IncytePD: 3871545 | Hs.140720 |
| | T-cell lymphomas 2 | [10q23-q24.1] | | |
| | prevent gsk-3-dependent | | | |
| | phosphorylation | | | |
| 161650 | KIAA0415 gene product | KIAA0415 | IncytePD: 2798872 | Hs.229950 |
| | | [7p22.2] | | |
| 168386 | nucleolar and coiled-body | NOLC1 | IncytePD: 1431819 | Hs.75337 |
| | phosphprotein 1 | [10] | | |
| 159906 | H2A histone family, member X | H2AFX | IncytePD: 1704168 | Hs.147097 |
| | | [11q23.2-q23.3] | | |
| 167906 | RAE1 (RNA export 1, S. pombe) | RAE1 | IncytePD: 2914719 | Hs.196209 |
| | homolog | [20q13.31] | | |
| | RNA export from the N | | | |
| 160486 | deltex (Drosophila) homolog 2 | DTX2 | IncytePD: 1691161 | Hs.89135 |
| | collagen type iii | [7q11.23] | | |
| 160678 | v-maf musculoaponeurotic | MAFG | IncytePD: 2956906 | Hs.252229 |
| | fibrosarcoma (avian) oncogene | [17q25] | | |
| | family, protein G | | | |
| | transcriptional regulator | | | |
| 159889 | fusion, derived from t(12;16) | FUS | IncytePD: 3036508 | Hs.99969 |
| | malignant liposarcoma | [16p11.2] | | |
| | DNA Sx atp-independent | | | |
| | annealing of complementary | | | |
| | single-stranded dnas | | | |
| 167553 | ligase I, DNA, ATP-dependent | LIG1 | IncytePD: 1841920 | Hs.1770 |
| | DNA excision repair process | [19q13.2-q13.3] | | |
| 163824 | uracil-DNA glycosylase | UNG | IncytePD: 1405652 | Hs.78853 |
| | DNA Base-excision repair | [12q23-q24.1] | | |
| 161012 | GCN1 (general control of amino- | GCN1L1 | IncytePD: 1699149 | Hs.75354 |
| | acid synthesis 1, yeast)-like 1 | [12q24.2] | | |
| 162006 | regenerating islet-derived 1 beta | REG1B | IncytePD: 2374294 | Hs.4158 |
| | (pancreatic stone protein, | [2p12] | | |
| | pancreatic thread protein) | | | |
| | brain and pancreas regeneration | | | |
| 161454 | serine protease inhibitor, Kunitz | SPINT1 | IncytePD: 2722572 | Hs.233950 |
| | type 1 | [15q13.3] | | |
| | Secreted S/Protease; proteolytic | | | |
| | activation of HGF | | | |
| 162510 | calcium/calmodulin-dependent | CAMKK2 | IncytePD: 557451 | Hs.108708 |
| | protein kinase kinase 2, beta | [12] | | |
| | S/T Protein kinase | | | |
| 163306 | Bloom syndrome | BLM | IncytePD: 2923082 | Hs.36820 |
| | DNA Repair | [15q26.1] | | |
| 160242 | RNA, U transporter 1 | RNUT1 | IncytePD: 1562658 | Hs.21577 |
| 164106 | glutamate rich WD repeat protein | GRWD | IncytePD: 1561867 | Hs.218842 |
| | GRWD | [19q13.33] | | |
| | RNA stability | | | |
| 165799 | MAD (mothers against | MADH3 | IncytePD: 1858365 | Hs.211578 |
| | decapentaplegic, Drosophila) | [15q21-q22] | | |
| | homolog 3 | | | |
| | TF, activated by tgf-beta | | | |
| 166574 | small nuclear RNA activating | SNAPC2 | IncytePD: 1445203 | Hs.78403 |
| | complex, polypeptide 2, 45 kD | [19p13.3-p13.2] | | |
| | RNA Processing | | | |
| 160441 | lymphotoxin beta receptor (TNFR | LTBR | IncytePD: 899102 | Hs.1116 |
| | superfamily, member 3) | [12p13] | | |
| | TNF family of receptors | | | |
| 168453 | transforming, acidic coiled-coil | TACC3 | IncytePD: 2056642 | Hs.104019 |
| | containing protein 3 | [4p16.3] | | |
| | Upregulated in Tumors | | | |
| 164244 | proteasome (prosome, macropain) | PSMC4 | IncytePD: 2806778 | Hs.211594 |
| | 26S subunit, ATPase, 4 | [19q13.11-q13.13] | | |
| 169564 | SWI/SNF related, matrix | SMARCD2 | IncytePD: 1890919 | Hs.250581 |
| | associated, actin dependent | [17q23-q24] | | |
| | regulator of chromatin, subfamily d, | | | |
| | member 2 | | | |
| | TF | | | |
| 161178 | basigin (OK blood group) | BSG | IncytePD: 2182907 | Hs.74631 |
| | Induces MMTP; p-regulated in | [19p13.3] | | |
| | gliomas | | | |
| 165614 | junction plakoglobin | JUP | IncytePD: 820580 | Hs.2340 |
| | | [17q21] | | |
| 168987 | HMT1 (hnRNP methyltransferase, | HRMT1L2 | IncytePD: 2888814 | Hs.20521 |
| | S. cerevisiae)-like 2 | [19q13.3] | | |
| | Protein methylation | | | |
| 167987 | ectonucleoside triphosphate | ENTPD1 | IncytePD: 1672749 | Hs.205353 |
| | diphosphohydrolase 1 | [10q24] | | |
| | ATP hydrolysis, Plt aggregation | | | |
| 163726 | complement component 3 | C3 | IncytePD: 1513989 | Hs.284394 |
| | | [19p13.3-p13.2] | | |
| 164642 | tyrosyl-tRNA synthetase | YARS | IncytePD: 1559756 | Hs.239307 |
| | | [1p34.3] | | |
| 160303 | Ets2 repressor factor | ERF | IncytePD: 2057547 | Hs.333069 |
| | | [19q13] | | |
| 161635 | G protein-coupled receptor | TYMSTR | IncytePD: 2610374 | Hs.34526 |
| | | [3p21] | | |
| 159859 | nuclear autoantigen | GS2NA | IncytePD: 1339241 | Hs.183105 |
| | wd REPEAT PROTEIN | [14q13-q21] | | |
| 161051 | MAP/microtubule affinity-regulating | MARK3 | IncytePD: 2395018 | Hs.172766 |
| | kinase 3 | [14q32.3] | | |
| | S/T Protein kinase | | | |
| 161835 | peroxisome biogenesis factor 10 | PEX10 | IncytePD: 3115936 | Hs.247220 |
| | | [1p36.11-1p36.33] | | |
| 165571 | annexin A3 | ANXA3 | IncytePD: 1920650 | Hs.1378 |
| | calcium-dependent phospholipid- | [4q13-q22] | | |
| | binding | | | |
| 164286 | nuclear factor of kappa light | NFKBIE | IncytePD: 2748942 | Hs.91640 |
| | polypeptide gene enhancer in B- | [6p21.1] | | |
| | cells inhibitor, epsilon | | | |
| 165786 | hyaluronoglucosaminidase 2 | HYAL2 | IncytePD: 1240748 | Hs.76873 |
| | Degrades glycosaminoglycans of | [3p21.3] | | |
| | the extracellular matrix | | | |
| 161620 | H4 histone family, member E | H4FE | IncytePD: 3728255 | Hs.278483 |
| | | [6p22-p21.3] | | |
| 168302 | Tax interaction protein 1 | TIP-1 | IncytePD: 1997792 | Hs.12956 |
| | 1 pdz/dhr domain | [17p13] | | |
| 160887 | pescadillo (zebrafish) homolog 1, | PES1 | IncytePD: 2758740 | Hs.13501 |
| | containing BRCT domain | [22q12.1] | | |
| | embrional dev | | | |
| 162419 | RAE1 (RNA export 1, S. pombe) | RAE1 | IncytePD: 588157 | Hs.196209 |
| | homolog | [20q13.31] | | |
| 169625 | replication factor C (activator 1) 4 | RFC4 | IncytePD: 1773638 | Hs.35120 |
| | (37 kD) | [3q27] | | |
| | DNA Sx/Repair | | | |
| 163425 | transcription elongation factor A | TCEA2 | IncytePD: 818568 | Hs.80598 |
| | (SII), 2 | [20] | | |
| 166359 | tubulin, beta polypeptide | TUBB | IncytePD: 3334367 | Hs.336780 |
| | Testis-specific | [6p21.3] | | |
| 161947 | translocase of inner mitochondrial | TIM17B | IncytePD: 1727491 | Hs.19105 |
| | membrane 17 homolog B (yeast) | [Xp11.23] | | |
| | Integral Mitoch. Expr. In | | | |
| | Neuroendocr Lung CA | | | |
| 162236 | KIAA0670 protein/acinus | KIAA0670 | IncytePD: 1968610 | Hs.227133 |
| | | [14q11.1] | | |
| 168426 | glioma pathogenesis-related | RTVP1 | IncytePD: 477045 | Hs.64639 |
| | protein | [12q15] | | |

Characteristics Of The Gene Expression Patterns In Pulmonary Neuroendocrine Tumors. The present invention permits investigation of whether expression of genes significantly altered in neuroendocrine tumors correlates with clinical behavior of these tumors. The results show that most of 198 selected genes could be assigned to major functional groups that have been previously implicated on cancer development (Table 3). In particular, decreased expression of genes that oppose cell survival pathway, such as BCL2 antagonist-killer, BAKI, and caspase 4, are common in all 3 types of neuroendocrine tumors, whereas TC and LCNEC have an additional >2.5-fold decrease in expression of BAD and TNF receptor-interacting kinase, RIPK1. These features indicate that these tumors lack opposing effects on BCL2, as contrasted to overexpression of BCL2, which leads to survival advantage in certain types of lymphomas (Cleary, M. L. et al. (1 986) “CLONING AND STRUCTURAL ANALYSIS OF cDNAS FOR BCL-2 AND A HYBRID BCL-2/IMMUNOGLOBULIN TRANSCRIPT RESULTING FROM THE T(14; 18) TRANSLOCATION,” Cell. 47(1):19-28).

Genes involved in regulation of cell-cell and extracellular matrix interactions, claudin 11 (CLDN11), contractin-2, (CNTN2), keratin 5 and 18 (KRT 5 and 18), calcium and integrin binding protein (SIP2-28), and junction plakoglobulin (JUP) are also suppressed in TC and LCNEC tumors, and, to a lesser degree, in SCLC. The dominant group of genes is involved in transcriptional regulation and DNA synthesis and repair. Decrease in expression of Bloom (BLM) is shared by TC and LCNEC, whereas DNA excision repair (ERCC1) and DNA ligase-1 (LIG) are suppressed in all tumor types. Other groups of genes manifesting decreased expression in all tumors are genes involved in cell cycle control (CDC34, p16/CDK inhibitor 2A), suppressor of MAPK pathway (dual specificity phosphatase, DUSP4), antioncogenes, such as epithin (ST14), and prohibitin, (PHB). Decreased expression of genes involved in microtubular assembly, beta tubulin polypepetide B (TUBB) in conjunction with overexpression of ATP-binding cassette protein (ABCG2) and gamma glutamyl hydrolase (GGH), could confer well-known resistance of these tumors to chemotherapy, specifically to taxol-related drugs. Decreased expression of genes associated with the ubiquitin pathway, such as proteasome subunit 26S (PSMC4), and proteasome activator subunit 3 (PSME3), correlates with potential resistance to newly developed proteasome inhibitors. The decrease in expression of these genes can affect NFκB activity, drug resistance and other functions in these tumors.

Only a fraction of genes identified herein is significantly over-expressed. Expression of a neuroendocrine peptide processing enzyme, carboxypeptidase E (CPE), inotropic glutamate receptor (GRIA2) and a complement component 5 are increased 4-6-fold in TC. In addition, TC has a modest increase in expression of the IL8 receptor B, IL8RB (1.61-fold), and that of the interleukin 6 signal transducer chain common to several interleukin receptors, gp130 (Oncostatin M, IL6ST), which is elevated at a mean of 1.34-fold in the 11 samples from TC. In contrast, LCNEC, have over 20 genes whose expression is above 1.9-fold or higher (FIGS. 3A and 3B). These gene products are increased specifically in LCNEC and included colony stimulating factor receptor (CSF2R), IL 13 receptor (IL13RA2), IL-8 receptor beta (IL8RB) as well as the IL 6 signal transducer, gp130 (Oncostatin M, IL6ST) and gamma-glutamyl hydrolase (GGH), which has been associated with drug resistance. In addition, LCNEC have a six-fold over-expression of a neuronal marker, P311, recently identified as a marker of aggressive gliomas. P311 may have a role in defining a metastatic/invasive potential in LCNEC. In contrast to LCNEC, analysis of SCLC shows only modest increased in 25 genes, none of which exceeded 1.5-fold increase. The lack of detection of over-expressed genes in SCLC reported herein could reflect a qualitative change in oncogenic mutations, such as p21^{ras}, p53 and others which are found in SCLC (Wistuba, I. I. et al. (2001) “MOLECULAR GENETICS OF SMALL CELL LUNG CARCINOMA,” Semin. Oncol. 28: 3-13) or due to limited number of samples used.

TABLE 3

| | |
|---|---|
| | Expression of Genes |
| | in Large Cell (LC), |
| | Small Cell (SC) and |
| Unique ID No. of | Typical Carcinoma |
| Gene | (TC) Cells |

| | | | | |
|---|---|---|---|---|
| Gene Family | (LOH) | LC | SC | TC |

Apoptosis

| | | | | |
|---|---|---|---|---|
| 167125 | Yes | 3.23 | 0.88 | 1.36 |
| 162623 | Yes | 0.23 | 0.51 | 0.13 |
| 160038 | Yes | 0.47 | 1.04 | 0.32 |
| 165813 | | 0.59 | 0.75 | 0.28 |
| 168079 | | 0.46 | 0.93 | 0.25 |
| 164799 | Yes | 1.2 | 0.73 | 0.64 |
| 160441 | | 0.37 | 0.49 | 0.18 |
| 161223 | | 0.2 | 0.71 | 0.11 |
| 166010 | | 0.45 | 0.99 | 0.28 |
| 167607 | | 0.4 | 0.81 | 0.23 |
| 166960 | | 0.17 | 0.37 | 0.09 |

Cell-Cell And ECM Interactions

| | | | | |
|---|---|---|---|---|
| 168700 | Yes | 1.91 | 0.82 | 1.69 |
| 168276 | | 1.61 | 0.63 | 1.21 |
| 162912 | | 0.82 | 0.7 | 1.27 |
| 161896 | | 2.12 | 0.75 | 1.04 |
| 159813 | | 1.99 | 0.83 | 1.22 |
| 166812 | | 0.93 | 0.78 | 0.78 |
| 165171 | | 0.3 | 0.16 | 0.05 |
| 166635 | | 0.18 | 0.63 | 0.11 |
| 161774 | Yes | 0.2 | 0.57 | 0.11 |
| 161211 | | 0.27 | 0.64 | 0.12 |
| 161948 | | 0.19 | 0.56 | 0.09 |
| 162734 | | 0.73 | 1.01 | 0.32 |
| 163690 | | 0.42 | 0.82 | 0.23 |
| 161955 | | 0.17 | 0.38 | 0.09 |
| 164206 | | 0.26 | 0.53 | 0.11 |
| 168642 | | 0.55 | 0.96 | 0.3 |
| 160486 | | 0.37 | 0.72 | 0.19 |
| 161178 | Yes | 0.52 | 1.05 | 0.36 |
| 165614 | Yes | 0.32 | 0.82 | 0.2 |
| 167987 | Yes | 0.58 | 1.03 | 0.32 |
| 165786 | | 0.56 | 0.94 | 0.35 |
| 164504 | | | | |

DNA Synthesis and Repair

| | | | | |
|---|---|---|---|---|
| 163306 | | 0.57 | 0.98 | 0.35 |
| 167135 | Yes | 0.34 | 0.63 | 0.2 |
| 160035 | | 0.21 | 0.72 | 0.11 |
| 160262 | | 0.19 | 0.58 | 0.12 |
| 161026 | | 0.54 | 0.78 | 0.28 |
| 159889 | | 0.33 | 0.79 | 0.22 |
| 167553 | Yes | 0.34 | 0.67 | 0.23 |
| 163824 | | 0.39 | 0.79 | 0.24 |
| 169625 | | 0.98 | 0.88 | 0.44 |

Cell Cycle

| | | | | |
|---|---|---|---|---|
| 167964 | | 0.15 | 0.33 | 0.08 |
| 160715 | Yes | 0.33 | 0.94 | 0.17 |
| 167180 | | 1.54 | 1.37 | 1.17 |
| 165052 | | 0.18 | 0.6 | 0.08 |
| 162391 | | 0.17 | 0.6 | 0.11 |
| 162631 | | 0.43 | 1.06 | 0.38 |
| 168638 | | 0.21 | 0.58 | 0.14 |

Anti-Oncogenes

| | | | | |
|---|---|---|---|---|
| 161058 | Yes | 0.72 | 1.25 | 0.39 |
| 165648 | | 0.31 | 0.6 | 0.19 |
| 169551 | | 0.47 | 0.8 | 0.26 |
| 160802 | | 0.16 | 0.44 | 0.09 |
| 161574 | Yes | 0.6 | 1.05 | 0.4 |

Oncogenes

| | | | | |
|---|---|---|---|---|
| 160429 | | 2.54 | 0.71 | 0.94 |
| 167948 | Yes | 0.61 | 1.16 | 0.28 |
| 159898 | Yes | 0.28 | 0.42 | 0.09 |
| 165799 | Yes | 0.53 | 0.67 | 0.27 |

Cytoskeleton/Migration

| | | | | |
|---|---|---|---|---|
| 160999 | Yes | 0.42 | 0.91 | 0.24 |
| 161754 | | 0.53 | 1.11 | 0.35 |
| 169071 | Yes | 0.3 | 0.72 | 0.21 |
| 167351 | | 0.39 | 0.69 | 0.26 |
| 162343 | | 0.33 | 0.67 | 0.17 |
| 162727 | Yes | 0.2 | 0.45 | 0.11 |
| 165784 | Yes | 1.46 | 0.69 | 1.96 |
| 160605 | | 5.94 | 0.84 | 1.06 |

Proteasome

| | | | | |
|---|---|---|---|---|
| 166328 | | 1.14 | 0.72 | 2.12 |
| 169691 | Yes | 0.15 | 0.34 | 0.09 |
| 168266 | Yes | 0.2 | 0.45 | 0.1 |
| 164244 | Yes | 0.43 | 0.67 | 0.22 |

Drug Resistance

| | | | | |
|---|---|---|---|---|
| 161117 | | 2.52 | 0.75 | 1.12 |
| 167738 | | 0.32 | 0.64 | 0.18 |
| 167505 | | 0.39 | 0.77 | 0.21 |
| 166359 | Yes | 0.46 | 0.64 | 0.28 |
| 167153 | | 6.27 | 1 | 1.31 |
| 168061 | | 1.32 | 0.64 | 1.23 |

Growth Factors/Receptors And Signal Transduction Enzymes

| | | | |
|---|---|---|---|
| 165576 | 1.93 | 0.66 | 1.34 |
| 169180 | 1.88 | 0.86 | 1.61 |
| 160617 | 3.57 | 0.86 | 0.93 |
| 164652 | 2.63 | 0.97 | 1.18 |
| 165617 | 2.9 | 0.73 | 1.32 |
| 169432 | 2.04 | 0.65 | 1.04 |
| 161391 | 0.43 | 0.83 | 0.25 |
| 164850 | 0.2 | 0.45 | 0.09 |
| 165483 | 0.33 | 0.98 | 0.23 |
| 162006 | 0.29 | 0.71 | 0.2 |
| 161454 | 0.58 | 0.99 | 0.39 |
| 168453 | 0.35 | 0.59 | 0.18 |
| 162220 | 0.34 | 0.76 | 0.25 |
| 160233 | 2.07 | 0.97 | 1.13 |

Neuronal Markers

| | | | | |
|---|---|---|---|---|
| 166807 | | | | |
| 159877 | | 1.39 | 0.93 | 5.89 |
| 162207 | Yes | 0.17 | 0.58 | 0.13 |
| 161948 | | 0.19 | 0.56 | 0.09 |
| 159898 | Yes | 0.28 | 0.42 | 0.09 |
| 160127 | Yes | 0.14 | 0.44 | 0.1 |
| 161955 | | 0.17 | 0.38 | 0.09 |
| 167591 | | 0.18 | 0.46 | 0.14 |
| 162006 | | 0.29 | 0.71 | 0.2 |
| 160887 | | 0.89 | 1.4 | 0.56 |
| 162247 | | | | |
| 165400 | | 1.7 | 0.76 | 0.82 |

RNA Synthesis, Processing and Transcription Factors

| | | | | |
|---|---|---|---|---|
| 161598 | | 0.82 | 0.96 | 2.59 |
| 169429 | | 4.52 | 0.8 | 1.18 |
| 165076 | | 0.96 | 0.81 | 1.53 |
| 167103 | | 1.7 | 0.72 | 1.34 |
| 169391 | Yes | 0.98 | 0.66 | 1.15 |
| 166789 | Yes | 1.76 | 0.75 | 1.07 |
| 168428 | Yes | | | |
| 165588 | | 1.11 | 0.8 | 0.57 |
| 164927 | | 0.51 | 1.65 | 1.4 |
| 160630 | Yes | 0.53 | 1.15 | 1.35 |
| 160367 | | 0.58 | 1.26 | 0.92 |
| 167931 | | 0.38 | 0.99 | 0.35 |
| 161533 | | 1.59 | 0.67 | 0.48 |
| 168110 | Yes | 0.35 | 0.8 | 0.21 |
| 161374 | Yes | 0.34 | 0.89 | 0.19 |
| 162587 | | 0.28 | 0.63 | 0.17 |
| 160467 | Yes | 0.17 | 0.44 | 0.12 |
| 160803 | Yes | 0.3 | 0.71 | 0.18 |
| 169254 | Yes | 0.29 | 0.6 | 0.16 |
| 160678 | | 0.48 | 0.94 | 0.29 |
| 160242 | | 0.59 | 0.83 | 0.31 |
| 164106 | Yes | 0.48 | 0.61 | 0.24 |
| 166574 | Yes | 0.47 | 0.89 | 0.25 |
| 169564 | | 0.25 | 0.48 | 0.15 |
| 164642 | | 0.69 | 0.92 | 0.27 |
| 162419 | | 0.59 | 1.03 | 0.44 |
| 163425 | | 0.95 | 0.86 | 0.44 |
| 160303 | Yes | 0.62 | 1.45 | 0.46 |
| 164573 | Yes | 2.23 | 0.82 | 1.37 |

Molecular Signature Of The Subtypes Of Pulmonary Neuroendocrine Tumors. The expression profile of genes significantly altered in neuroendocrine tumors was examined to determine whether such information could be used to differentiate among each subtype of pulmonary neuroendocrine tumors. To establish a signature list for each tumor type, the relative expression ratio between TC, LCNEC and SCLC is employed. Table 4 shows the extent of expression of such a signature list, and provides the ratio of expression. In Table 4, TC/SC denotes genes exhibiting higher levels of expression in TC cells than in SC cells; SC/TC denotes genes exhibiting higher levels of expression in SC cells than in TC cells. Data for TC/LC, LC/TC, SC/LC, and LC/SC are similarly provided. This form of statistical analysis is independent of the reference value and, therefore, can be used for future studies. Using a ratio of 1.9 or higher, it is found that TC had 15 genes whose expression distinguished these tumors from SCLC, and 12 from LCNEC. In contrast, 134 genes are higher in SCLC than in TC and 97 higher than in LCNEC (Table 4). The difference between expression of genes in LCNEC from SCLC is encompassed within 34 genes. Thus, cDNA microarray analysis derived expression profile obtained using a cell line as a reference can be used to develop a molecular signature algorithm which may be useful for differential diagnosis of these tumors.

TABLE 4
Molecular Signature of Neuroendocrine Tumors

| | | | |
|---|---|---|---|
| Unique ID | Observed | | Observed |
| No. of Gene | Expression | Ratio | Expression |

TC/SC

| | TC | SC | TC/SC | Normal Cells |
|---|---|---|---|---|
| 159877 | 5.89 | 0.93 | 6.33 | |
| 167158 | 6.52 | 1.16 | 5.62 | |
| 166807 | 4.46 | 0.81 | 5.51 | |
| 163031 | 3.15 | 1.02 | 3.09 | 1.06 |
| 166328 | 2.12 | 0.72 | 2.94 | |
| 165784 | 1.96 | 0.69 | 2.84 | |
| 161598 | 2.59 | 0.96 | 2.70 | |
| 165393 | 1.98 | 0.96 | 2.10 | |
| 168700 | 1.69 | 0.82 | 2.06 | |
| 165192 | 1.56 | 0.76 | 2.05 | |
| 165576 | 1.34 | 0.66 | 2.03 | |
| 168061 | 1.23 | 0.64 | 1.92 | |
| 168276 | 1.21 | 0.63 | 1.92 | |
| 165076 | 1.53 | 0.81 | 1.89 | |
| 169180 | 1.61 | 0.86 | 1.87 | |

SC/TC

| | SC | TC | SC/TC | Normal Cells |
|---|---|---|---|---|
| 165052 | 0.60 | 0.08 | 7.50 | 0.50 |
| 161163 | 0.53 | 0.08 | 6.63 | 0.40 |
| 160035 | 0.72 | 0.11 | 6.55 | 0.50 |
| 161223 | 0.71 | 0.11 | 6.45 | 0.40 |
| 161948 | 0.56 | 0.09 | 6.22 | 0.22 |
| 166635 | 0.63 | 0.11 | 5.73 | 0.40 |
| 165583 | 0.28 | 0.05 | 5.60 | 0.20 |
| 160715 | 0.94 | 0.17 | 5.53 | 0.67 |
| 162391 | 0.60 | 0.11 | 5.45 | 0.35 |
| 161244 | 0.38 | 0.07 | 5.43 | 0.20 |
| 161211 | 0.64 | 0.12 | 5.33 | 0.35 |
| 161774 | 0.57 | 0.11 | 5.18 | 0.40 |
| 166195 | 0.56 | 0.11 | 5.09 | 0.30 |
| 164850 | 0.45 | 0.09 | 5.00 | 0.38 |
| 160802 | 0.44 | 0.09 | 4.89 | |
| 161643 | 1.16 | 0.24 | 4.83 | 0.80 |
| 160262 | 0.58 | 0.12 | 4.83 | |
| 164206 | 0.53 | 0.11 | 4.82 | 0.40 |
| 164586 | 0.48 | 0.10 | 4.80 | 0.35 |
| 165039 | 0.19 | 0.04 | 4.75 | 0.10 |
| 161374 | 0.89 | 0.19 | 4.68 | 0.55 |
| 159898 | 0.42 | 0.09 | 4.67 | 0.26 |
| 160102 | 1.07 | 0.23 | 4.65 | |
| 164646 | 0.69 | 0.15 | 4.60 | 0.42 |
| 163787 | 0.81 | 0.18 | 4.50 | 0.50 |
| 168266 | 0.45 | 0.10 | 4.50 | |
| 161790 | 0.45 | 0.10 | 4.50 | |
| 162207 | 0.58 | 0.13 | 4.46 | 0.55 |
| 160127 | 0.44 | 0.10 | 4.40 | 0.40 |
| 160323 | 0.43 | 0.10 | 4.30 | 0.30 |
| 165483 | 0.98 | 0.23 | 4.26 | 0.73 |
| 161955 | 0.38 | 0.09 | 4.22 | |
| 167948 | 1.16 | 0.28 | 4.14 | 1.86 |
| 168638 | 0.58 | 0.14 | 4.14 | |
| 167964 | 0.33 | 0.08 | 4.13 | 0.23 |
| 166960 | 0.37 | 0.09 | 4.11 | 0.25 |
| 161954 | 0.78 | 0.19 | 4.11 | 0.20 |
| 165614 | 0.82 | 0.20 | 4.10 | 0.50 |
| 162727 | 0.45 | 0.11 | 4.09 | 0.25 |
| 167116 | 0.32 | 0.08 | 4.00 | |
| 160803 | 0.71 | 0.18 | 3.94 | 0.50 |
| 162343 | 0.67 | 0.17 | 3.94 | 0.62 |
| 163682 | 0.59 | 0.15 | 3.93 | |
| 162623 | 0.51 | 0.13 | 3.92 | 0.35 |
| 166914 | 0.61 | 0.16 | 3.81 | |
| 168110 | 0.80 | 0.21 | 3.81 | |
| 160999 | 0.91 | 0.24 | 3.79 | 0.60 |
| 160486 | 0.72 | 0.19 | 3.79 | 0.50 |
| 160275 | 0.53 | 0.14 | 3.79 | |
| 169691 | 0.34 | 0.09 | 3.78 | |
| 165790 | 0.45 | 0.12 | 3.75 | 0.30 |
| 169254 | 0.60 | 0.16 | 3.75 | |
| 168079 | 0.93 | 0.25 | 3.72 | 0.56 |
| 162587 | 0.63 | 0.17 | 3.71 | 0.55 |
| 162244 | 0.74 | 0.20 | 3.70 | 0.70 |
| 167505 | 0.77 | 0.21 | 3.67 | |
| 160467 | 0.44 | 0.12 | 3.67 | 0.30 |
| 161012 | 0.73 | 0.20 | 3.65 | 0.55 |
| 159889 | 0.79 | 0.22 | 3.59 | 0.55 |
| 163690 | 0.82 | 0.23 | 3.57 | 0.50 |
| 166574 | 0.89 | 0.25 | 3.56 | 0.62 |
| 167738 | 0.64 | 0.18 | 3.56 | 0.51 |
| 167706 | 0.64 | 0.18 | 3.56 | |
| 162006 | 0.71 | 0.20 | 3.55 | 0.31 |
| 166010 | 0.99 | 0.28 | 3.54 | 0.55 |
| 167607 | 0.81 | 0.23 | 3.52 | 0.82 |
| 159906 | 0.62 | 0.18 | 3.44 | 0.30 |
| 162150 | 1.10 | 0.32 | 3.44 | 0.60 |
| 169071 | 0.72 | 0.21 | 3.43 | |
| 162178 | 0.24 | 0.07 | 3.43 | 0.20 |
| 164642 | 0.92 | 0.27 | 3.41 | 0.40 |
| 167170 | 0.88 | 0.26 | 3.38 | 0.52 |
| 168386 | 0.81 | 0.24 | 3.38 | |
| 167223 | 0.87 | 0.26 | 3.35 | 0.65 |
| 161391 | 0.83 | 0.25 | 3.32 | 0.70 |
| 167906 | 0.63 | 0.19 | 3.32 | |
| 160565 | 0.56 | 0.17 | 3.29 | 0.56 |
| 163824 | 0.79 | 0.24 | 3.29 | |
| 167591 | 0.46 | 0.14 | 3.29 | |
| 168453 | 0.59 | 0.18 | 3.28 | |
| 161794 | 0.95 | 0.29 | 3.28 | 0.74 |
| 163726 | 1.21 | 0.37 | 3.27 | 0.90 |
| 160038 | 1.04 | 0.32 | 3.25 | 0.63 |
| 160678 | 0.94 | 0.29 | 3.24 | |
| 167987 | 1.03 | 0.32 | 3.22 | |
| 164504 | 0.77 | 0.24 | 3.21 | 0.80 |
| 161058 | 1.25 | 0.39 | 3.21 | |
| 168642 | 0.96 | 0.30 | 3.20 | |
| 169564 | 0.48 | 0.15 | 3.20 | |
| 165171 | 0.16 | 0.05 | 3.20 | |
| 161754 | 1.11 | 0.35 | 3.17 | 0.60 |
| 165648 | 0.60 | 0.19 | 3.16 | 0.48 |
| 162734 | 1.01 | 0.32 | 3.16 | 0.65 |
| 160303 | 1.45 | 0.46 | 3.15 | 1.30 |
| 167135 | 0.63 | 0.20 | 3.15 | |
| 160098 | 0.91 | 0.29 | 3.14 | 0.50 |
| 169551 | 0.80 | 0.26 | 3.08 | |
| 164244 | 0.67 | 0.22 | 3.05 | |
| 162220 | 0.76 | 0.25 | 3.04 | 0.60 |
| 164286 | 0.94 | 0.31 | 3.03 | |
| 161635 | 1.06 | 0.35 | 3.03 | 0.80 |
| 167713 | 0.77 | 0.26 | 2.96 | |
| 163276 | 0.47 | 0.16 | 2.94 | |
| 161178 | 1.05 | 0.36 | 2.92 | 0.60 |
| 167553 | 0.67 | 0.23 | 2.91 | |
| 163921 | 0.52 | 0.18 | 2.89 | 0.55 |
| 167931 | 0.99 | 0.35 | 2.83 | |
| 160938 | 0.82 | 0.29 | 2.83 | 0.50 |
| 163306 | 0.98 | 0.35 | 2.80 | 0.50 |
| 161650 | 1.23 | 0.44 | 2.80 | |
| 162631 | 1.06 | 0.38 | 2.79 | |
| 161026 | 0.78 | 0.28 | 2.79 | |
| 162571 | 1.11 | 0.40 | 2.78 | 0.80 |
| 160478 | 1.07 | 0.39 | 2.74 | |
| 160441 | 0.49 | 0.18 | 2.72 | 0.42 |
| 165786 | 0.95 | 0.35 | 2.71 | 0.60 |
| 165571 | 0.84 | 0.31 | 2.71 | 0.80 |
| 161620 | 0.84 | 0.31 | 2.71 | 0.80 |
| 165813 | 0.75 | 0.28 | 2.68 | 0.70 |
| 160242 | 0.83 | 0.31 | 2.68 | |
| 168302 | 0.88 | 0.33 | 2.67 | |
| 167351 | 0.69 | 0.26 | 2.65 | 0.40 |
| 168987 | 0.79 | 0.30 | 2.63 | |
| 161574 | 1.05 | 0.40 | 2.63 | |
| 162510 | 0.91 | 0.35 | 2.60 | 0.72 |
| 164106 | 0.61 | 0.24 | 2.54 | 0.50 |
| 161454 | 0.99 | 0.39 | 2.54 | 0.60 |
| 160887 | 1.40 | 0.56 | 2.50 | 1.24 |
| 165799 | 0.67 | 0.27 | 2.48 | 0.55 |
| 162419 | 1.03 | 0.44 | 2.34 | 0.80 |
| 166359 | 0.64 | 0.28 | 2.29 | |
| 169625 | 0.88 | 0.44 | 2.00 | |
| 168426 | 1.09 | 0.55 | 1.98 | |
| 163425 | 0.86 | 0.44 | 1.95 | 0.80 |

TC/LC

| | TC | LC | TC/LC | Normal Cells |
|---|---|---|---|---|
| 167158 | 6.52 | 0.87 | 7.49 | |
| 159877 | 5.89 | 1.39 | 4.24 | |
| 166807 | 4.46 | 1.11 | 4.02 | |
| 161598 | 2.59 | 0.82 | 3.16 | |
| 164927 | 1.40 | 0.51 | 2.75 | |
| 163031 | 3.15 | 1.22 | 2.58 | |
| 160630 | 1.35 | 0.53 | 2.55 | |
| 162247 | 1.40 | 0.67 | 2.09 | |
| 167219 | 1.16 | 0.57 | 2.04 | |
| 163475 | 1.17 | 0.60 | 1.95 | |
| 163762 | 1.04 | 0.54 | 1.93 | |
| 166328 | 2.12 | 1.14 | 1.86 | |

LC/TC

| | LC | TC | LC/TC | Normal Cells |
|---|---|---|---|---|
| 165400 | 1.70 | 0.82 | 2.07 | |
| 164850 | 0.20 | 0.09 | 2.22 | |
| 164868 | 2.39 | 1.16 | 2.06 | |
| 161533 | 1.59 | 0.48 | 3.31 | |
| 160957 | 3.20 | 1.16 | 2.76 | |
| 169429 | 4.52 | 1.18 | 3.83 | |
| 169432 | 2.04 | 1.04 | 1.96 | |
| 165583 | 0.10 | 0.05 | 2.00 | 0.20 |
| 165617 | 2.90 | 1.32 | 2.20 | |
| 168987 | 0.60 | 0.30 | 2.00 | |
| 161709 | 1.89 | 0.79 | 2.39 | |
| 169625 | 0.98 | 0.44 | 2.23 | |
| 165799 | 0.53 | 0.27 | 1.96 | 0.55 |
| 161896 | 2.12 | 1.04 | 2.04 | |
| 165813 | 0.59 | 0.28 | 2.11 | 0.70 |
| 162571 | 1.32 | 0.40 | 3.30 | 0.80 |
| 161948 | 0.19 | 0.09 | 2.11 | 0.22 |
| 167116 | 0.18 | 0.08 | 2.25 | |
| 167125 | 3.23 | 1.36 | 2.38 | |
| 167153 | 6.27 | 1.31 | 4.79 | |
| 162734 | 0.73 | 0.32 | 2.28 | 0.60 |
| 163425 | 0.95 | 0.44 | 2.16 | 0.80 |
| 164106 | 0.48 | 0.24 | 2.00 | 0.50 |
| 160237 | 3.50 | 1.38 | 2.54 | |
| 164206 | 0.26 | 0.11 | 2.36 | |
| 164244 | 0.43 | 0.22 | 1.95 | |
| 168266 | 0.20 | 0.10 | 2.00 | |
| 160429 | 2.54 | 0.94 | 2.70 | 0.94 |
| 159898 | 0.28 | 0.09 | 3.11 | 0.25 |
| 160441 | 0.37 | 0.18 | 2.06 | 0.42 |
| 167713 | 0.64 | 0.26 | 2.46 | |
| 165052 | 0.18 | 0.08 | 2.25 | 0.50 |
| 159906 | 0.42 | 0.18 | 2.33 | 0.30 |
| 161117 | 2.52 | 1.12 | 2.25 | 1.12 |
| 161163 | 0.18 | 0.08 | 2.25 | 0.35 |
| 160565 | 0.45 | 0.17 | 2.65 | 0.50 |
| 164504 | 0.51 | 0.24 | 2.13 | 0.80 |
| 165171 | 0.30 | 0.05 | 6.00 | |
| 161211 | 0.27 | 0.12 | 2.25 | 0.35 |
| 160605 | 5.94 | 1.06 | 5.60 | 0.78 |
| 160617 | 3.57 | 0.93 | 3.84 | 0.90 |
| 167906 | 0.40 | 0.19 | 2.11 | 0.80 |
| 167948 | 0.61 | 0.28 | 2.18 | |
| 164642 | 0.69 | 0.27 | 2.56 | 0.45 |
| 164646 | 0.39 | 0.15 | 2.60 | 0.42 |
| 164652 | 2.63 | 1.18 | 2.23 | |

SC/LC

| | SC | LC | SC/LC | Normal Cells |
|---|---|---|---|---|
| 161244 | 0.38 | 0.10 | 3.80 | 0.20 |
| 161223 | 0.71 | 0.20 | 3.55 | 0.40 |
| 162391 | 0.60 | 0.17 | 3.53 | 0.35 |
| 166635 | 0.63 | 0.18 | 3.50 | 0.40 |
| 160035 | 0.72 | 0.21 | 3.43 | 0.50 |
| 162207 | 0.58 | 0.17 | 3.41 | 0.55 |
| 165052 | 0.60 | 0.18 | 3.33 | 0.50 |
| 161954 | 0.78 | 0.24 | 3.25 | 0.20 |
| 164927 | 1.65 | 0.51 | 3.24 | |
| 160127 | 0.44 | 0.14 | 3.14 | 0.47 |
| 160262 | 0.58 | 0.19 | 3.05 | |
| 161643 | 1.16 | 0.39 | 2.97 | 0.80 |
| 165483 | 0.98 | 0.33 | 2.97 | 0.73 |
| 166195 | 0.56 | 0.19 | 2.95 | 0.30 |
| 161948 | 0.56 | 0.19 | 2.95 | 0.22 |
| 161163 | 0.53 | 0.18 | 2.94 | 0.35 |
| 167223 | 0.87 | 0.30 | 2.90 | 0.65 |
| 161774 | 0.57 | 0.20 | 2.85 | 0.45 |
| 160715 | 0.94 | 0.33 | 2.85 | 0.67 |
| 164586 | 0.48 | 0.17 | 2.82 | 0.35 |
| 161790 | 0.45 | 0.16 | 2.81 | |
| 165583 | 0.28 | 0.10 | 2.80 | 0.20 |
| 168638 | 0.58 | 0.21 | 2.76 | 0.58 |
| 160802 | 0.44 | 0.16 | 2.75 | |
| 160102 | 1.07 | 0.39 | 2.74 | |
| 165039 | 0.19 | 0.07 | 2.71 | 0.10 |
| 163762 | 1.44 | 0.54 | 2.67 | |
| 161374 | 0.89 | 0.34 | 2.62 | 0.55 |
| 163787 | 0.81 | 0.31 | 2.61 | 0.50 |
| 161012 | 0.73 | 0.28 | 2.61 | 0.55 |
| 167931 | 0.99 | 0.38 | 2.61 | |
| 160467 | 0.44 | 0.17 | 2.59 | 0.30 |
| 165614 | 0.82 | 0.32 | 2.56 | 0.50 |
| 167591 | 0.46 | 0.18 | 2.56 | |
| 165790 | 0.45 | 0.18 | 2.50 | 0.30 |
| 162244 | 0.74 | 0.30 | 2.47 | 0.70 |
| 162631 | 1.06 | 0.43 | 2.47 | |
| 161635 | 1.06 | 0.43 | 2.47 | 0.80 |
| 162006 | 0.71 | 0.29 | 2.45 | 0.31 |
| 162247 | 1.62 | 0.67 | 2.42 | |
| 169071 | 0.72 | 0.30 | 2.40 | |
| 159889 | 0.79 | 0.33 | 2.39 | 0.55 |
| 160323 | 0.43 | 0.18 | 2.39 | 0.30 |
| 161211 | 0.64 | 0.27 | 2.37 | 0.35 |
| 160803 | 0.71 | 0.30 | 2.37 | 0.55 |
| 160303 | 1.45 | 0.62 | 2.34 | 1.00 |
| 161794 | 0.95 | 0.41 | 2.32 | 0.70 |
| 168110 | 0.80 | 0.35 | 2.29 | |
| 167706 | 0.64 | 0.28 | 2.29 | |
| 169691 | 0.34 | 0.15 | 2.27 | |
| 168386 | 0.81 | 0.36 | 2.25 | |
| 162587 | 0.63 | 0.28 | 2.25 | |
| 168266 | 0.45 | 0.20 | 2.25 | |
| 164850 | 0.45 | 0.20 | 2.25 | 0.38 |
| 162727 | 0.45 | 0.20 | 2.25 | 0.25 |
| 162220 | 0.76 | 0.34 | 2.24 | 0.60 |
| 161955 | 0.38 | 0.17 | 2.24 | |
| 162623 | 0.51 | 0.23 | 2.22 | 0.36 |
| 160038 | 1.04 | 0.47 | 2.21 | |
| 167964 | 0.33 | 0.15 | 2.20 | |
| 166010 | 0.99 | 0.45 | 2.20 | 0.55 |
| 167170 | 0.88 | 0.40 | 2.20 | 0.52 |
| 167219 | 1.25 | 0.57 | 2.19 | |
| 163682 | 0.59 | 0.27 | 2.19 | |
| 162178 | 0.24 | 0.11 | 2.18 | 0.20 |
| 166960 | 0.37 | 0.17 | 2.18 | 0.25 |
| 160367 | 1.26 | 0.58 | 2.17 | |
| 160630 | 1.15 | 0.53 | 2.17 | |
| 160999 | 0.91 | 0.42 | 2.17 | 0.60 |
| 160275 | 0.53 | 0.25 | 2.12 | |
| 161754 | 1.11 | 0.53 | 2.09 | 0.60 |
| 163921 | 0.52 | 0.25 | 2.08 | 0.55 |
| 169254 | 0.60 | 0.29 | 2.07 | 0.28 |
| 164206 | 0.53 | 0.26 | 2.04 | 0.40 |
| 166914 | 0.61 | 0.30 | 2.03 | |
| 162343 | 0.67 | 0.33 | 2.03 | 0.62 |
| 163824 | 0.79 | 0.39 | 2.03 | 0.65 |
| 167607 | 0.81 | 0.40 | 2.03 | |
| 160098 | 0.91 | 0.45 | 2.02 | 0.50 |
| 168079 | 0.93 | 0.46 | 2.02 | 0.56 |
| 161178 | 1.05 | 0.52 | 2.02 | 0.60 |
| 160938 | 0.82 | 0.41 | 2.00 | 0.50 |
| 167738 | 0.64 | 0.32 | 2.00 | 0.51 |
| 167505 | 0.77 | 0.39 | 1.97 | |
| 159859 | 1.44 | 0.73 | 1.97 | 0.90 |
| 167553 | 0.67 | 0.34 | 1.97 | |
| 162150 | 1.10 | 0.56 | 1.96 | |
| 160678 | 0.94 | 0.48 | 1.96 | |
| 163690 | 0.82 | 0.42 | 1.95 | 0.50 |
| 160486 | 0.72 | 0.37 | 1.95 | 0.50 |
| 160478 | 1.07 | 0.55 | 1.95 | |
| 165648 | 0.60 | 0.31 | 1.94 | |
| 161391 | 0.83 | 0.43 | 1.93 | 0.70 |
| 169564 | 0.48 | 0.25 | 1.92 | |
| 167948 | 1.16 | 0.61 | 1.90 | |
| 166574 | 0.89 | 0.47 | 1.89 | |
| 167135 | 0.63 | 0.34 | 1.85 | |

LC/SC

| | LC | SC | LC/SC | Normal Cells |
|---|---|---|---|---|
| 165393 | 2.66 | 0.96 | 2.77 | |
| 168700 | 1.91 | 0.82 | 2.33 | |
| 169384 | 2.28 | 0.77 | 2.96 | |
| 165400 | 1.70 | 0.76 | 2.24 | |
| 161533 | 1.59 | 0.67 | 2.37 | 1.00 |
| 160957 | 3.20 | 0.77 | 4.16 | |
| 169429 | 4.52 | 0.80 | 5.65 | |
| 169432 | 2.04 | 0.65 | 3.14 | |
| 165576 | 1.93 | 0.66 | 2.92 | |
| 165617 | 2.90 | 0.73 | 3.97 | |
| 161709 | 1.89 | 0.95 | 1.99 | |
| 165784 | 1.46 | 0.69 | 2.12 | |
| 162475 | 2.00 | 1.06 | 1.89 | |
| 161896 | 2.12 | 0.75 | 2.83 | |
| 167103 | 1.70 | 0.72 | 2.36 | |
| 167125 | 3.23 | 0.88 | 3.67 | |
| 167153 | 6.27 | 1.00 | 6.27 | |
| 167316 | 1.94 | 0.88 | 2.20 | |
| 166789 | 1.76 | 0.75 | 2.35 | |
| 168061 | 1.32 | 0.64 | 2.06 | |
| 160233 | 2.07 | 0.97 | 2.13 | |
| 160237 | 3.50 | 0.92 | 3.80 | |
| 168141 | 2.51 | 0.95 | 2.64 | |
| 168169 | 2.78 | 1.17 | 2.38 | |
| 168276 | 1.61 | 0.63 | 2.56 | |
| 159813 | 1.99 | 0.83 | 2.40 | |
| 160429 | 2.54 | 0.71 | 3.58 | 0.90 |
| 161117 | 2.52 | 0.75 | 3.36 | |
| 165171 | 0.30 | 0.16 | 1.88 | |
| 164573 | 2.23 | 0.82 | 2.72 | |
| 160605 | 5.94 | 0.84 | 7.07 | 0.78 |
| 160617 | 3.57 | 0.86 | 4.15 | 0.90 |
| 169180 | 1.88 | 0.86 | 2.19 | |
| 164652 | 2.63 | 0.97 | 2.71 | |

Correlation Between Gene Expression Profiles And Genomic Imbalance. To compare the results obtained from cDNA array expression in accordance with the present invention with previously available information on genomic imbalances in neuroendocrine tumors, a search of the literature for published data on comparative genomic hybridization (CGH) and loss of heterozygosity (LOH) in neuroendocrine tumors was conducted. It was found that, among 198 genes identified by the Class Comparison (F-test) analysis, over ninety percent of genes with significant changes in LCNEC, and over 80% of genes from SCLC and TC, had previously been reported to have chromosomal imbalances by gain or loss (CGH) or to be associated with LOH (Table 5). Loss of chromosomal material by LOH closely correlated with genes whose expression significantly decreased in our analysis. Deletions of several genes, such as cyclin-dependent kinase inhibitor (CDKN2A, 9p21) and multiple endocrine neoplasia 1 (MEN1, 11q13) have been studied extensively in pulmonary neuroendocrine tumors (Oliveira, A. M. et al. (2001) “FAMILIAL PULMONARY CARCINOID TUMORS,” Cancer 91:2104-2109; Debelenko, L. V. et al. (2000) “MENi gene mutation analysis of high-grade neuroendocrine lung carcinoma,” Genes Chromosomes Cancer. 28:58-65). However, several genes whose expression has been found to be decreased herein were previously reported to have a gain of chromosomal material by CGH. These include BAK, excision repair cross-complement (ERCC1), DNA ligase (LIG1), tubulin beta (TUBB) and others (Table 2).

Of interest, none of the genes which encode for growth factor/receptors identified herein have been reported by LOH. However, loss of genetic material by CGH in these genes has been reported. The potential loss of repressor activity in the promoter regions of these genes may result in their over-expression as detected herein. In sum, the expression profiling of significantly altered genes derived from microarray data reported herein closely correlates with chromosomal imbalances reported by LOH but not by CGH.

### EXAMPLE 3

### Analysis of Gene Expression Profiles

Analysis of clusters of differentially expressed mRNAs from 9,984 human transcripts assigned to each subtype of neuroendocrine tumors identified multiple genes (198 genes with a probability of 0.004) exhibiting differential expression. This highly selected group of genes contained valuable information which correlated with biological behavior of these tumors. The identified genes are involved in regulation of apoptosis, cell-cell and cell-matrix interactions, cell cycle, DNA synthesis and repair, drug resistance, RNA synthesis and processing, receptors and growth factors. Previous studies using microarray analysis of lymphomas (Dodson, J. M. et al. (2002) “QUANTITATIVE ASSESSMENT OF FILTER-BASED cDNA MICROARRAYS: GENE EXPRESSION PROFILES OF HUMAN T-LYMPHOMA CELL LINES,” Bioinformatics 18:953-960; Ramaswamy, S. et al. (2001) MULTICLASS CANCER DIAGNOSIS USING TUMOR GENE EXPRESSION SIGNATURES,” Proc Natl Acad Sci U S A. 98(26):15149-15154), gastrointestinal (Hippo, Y. et al. (2002) “GLOBAL GENE EXPRESSION ANALYSIS OF GASTRIC CANCER BY OLIGONUCLEOTIDE MICROARRAYS,” Cancer Res. 62(l):233-240; Selaru, F. M. et al (2002) “ARTIFICIAL NEURAL NETWORKS DISTINGUISH AMONG SUBTYPES OF NEOPLASTIC COLORECTAL LESIONS,” Gastroenterology 122:606-613), ovarian (Ramaswamy, S. et al. (2001) MULTICLASS CANCER DIAGNOSIS USING TUMOR GENE EXPRESSION SIGNATURES,” Proc Nati Acad Sci USA. 98(26): 15149-15154), and other types of human tumors found that over-expression of specific genes is a prominent feature that facilitated the molecular classification of these tumors. In contrast, a significant decrease in expression in the majority of the selected genes was found. One of the major survival pathways is regulated by protection of the mitochondrial membrane by BCL2 which is frequently over-expressed in tumor cells (Cleary, M. L. et al. (1986) “CLONING AND STRUCTURAL ANALYSIS OF cDNAS FOR BCL-2 AND A HYBRID BCL-2/IMMUNOGLOBULIN TRANSCRIPT RESULTING FROM THE T(14; 18) TRANSLOCATION,” Cell. 47(1): 19-28). Decreased expression of BCL2 antagonists, BAD and BAK1 was observed in samples from TC and LCNEC. This feature may provide survival advantage without the need for over-expression of BCL2 as occurs in certain types of lymphomas. BAD and BAK1 are located on chromosomes 11q13 and 6p21, respectively, which are in the regions of loss of heterozygosity (LOH) in neuroendocrine tumors (Hofmann, W. K. (2002) “RELATION BETWEEN RESISTANCE OF PHILADELPHIA-CHROMOSOME-POSITIVE ACUTE LYMPHOBLASTIC LEUKAEMIA TO THE TYROSINE KINASE INHIBITOR STI571 AND GENE-EXPRESSION PROFILES: A GENE-EXPRESSION STUDY,” Lancet 359:481-486). Expression of BAK was further suppressed in TC and LCNEC below the level expected for LOH which suggests an additional regulatory mechanism. Interestingly, gain of chromosomal material in 6p21 was reported in LCNEC by CGH (Michelland, S. et al. (1999) “COMPARISON OF CHROMOSOMAL IMBALANCES IN NEUROENDOCRINE AND NON-SMALL-CELL LUNG CARCINOMAS,” Cancer Genet Cytogenet 114:22-30). Suppression of other apoptosis-promoting genes, such as caspase 4 (CASP4), may also provide survival advantage and has not been previously reported in Neuroendocrine tumors. Loss of expression of many genes which regulate cell-cell and cell-matrix interactions as well as DNA and RNA synthesis and repair were apparent in all tumor types (Table 2). Table 2 shows representative deregulated genes classified by function. Genes selected by F-test with probability of <0.004 were genes assigned to functional categories and compared with the published comparative genomic hybridization (CGH) results (Michelland, S. et al. (1999) “COMPARISON OF CHROMOSOMAL IMBALANCES IN NEUROENDOCRINE AND NON-SMALL-CELL LUNG CARCINOMAS,” Cancer Genet Cytogenet 114:22-30; Lui, W.-O. et al. (2001) “HIGH LEVEL AMPLIFICATION OF 1P32-33 AND 2p22-24 IN SMALL CELL LUNG CARCINOMAS” Intl. J. Oncol. 19:451-457; Ullmann, R., et al. (2001) “CHROMOSOMAL ABERRATIONS IN A SERIES OF LARGE-CELL NEUROENDOCRINE CARCINOMAS: UNEXPECTED DIVERGENCE FROM SMALL-CELL CARCINOMA OF THE LUNG,” Hum Pathol. 32:1059-63; Walch, A. K. et al. (1998) “TYPICAL AND ATYPICAL CARCINOID TUMORS OF THE LUNG ARE CHARACTERIZED BY 11Q DELETIONS AS DETECTED BY COMPARATIVE GENOMIC HYBRIDIZATION” Am J Pathol. 153:1089-98).

In the table, SC denotes small cell; LC denotes large cell neuroendocrine carcinoma; and TC denotes typical carcinoid.

Most studies performed to-date compare tumor samples with cDNA from normal tissues of an individual patient, pooled normal tissues or pooled cell lines as reference. To illustrate the invention, RNA from a single human cell line derived from normal bronchial epithelium, BEAS-2B (Amstad, P. et al. (1988) “NEOPLASTIC TRANSFORMATION OF A HUMAN BRONCHIAL EPITHELIAL CELL LINE BY A RECOMBINANT RETROVIRUS ENCODING VIRAL HARVEY RAS,” Mol Carcinog. 1988 1:151-60), was used as a reference RNA. This cell line has minimal chromosomal rearrangements in early passages and neuroendocrine tumor features (Lee, B. H et al. (1998) “IN VITRO CHROMOSOME ABERRATION ASSAY USING HUMAN BRONCHIAL EPITHELIAL CELLS,” J. Toxicol Environ. Health A. 55:325-9). Thus, the data indicate that accurate classification of neuroendocrine tumors can be achieved by comparing gene expression profiles of tumors to a single cell line derived from the same cell type. This method is applicable to analysis of tumor-derived gene expression profiles from other organs, such as brain, where availability of normal tissue is limited.

In addition to suppression of the apoptotic pathway, only LCNEC tumors had increased expression (2-6-fold) of several receptors and growth factors. Increased expression of PDGFRB in conjunction with suppression of PDGFA-associated protein, which can down regulate the activity of PDGFA, could result in additional proliferative signal and contribute to the aggressive behavior of this tumor. In addition, high expression of an adhesion plaque-associated protein, P311, which has been recently identified as a glioblastoma invasion gene (Mariani, L. et al. (200 1) “IDENTIFICATION AND VALIDATION OF P311 AS A GLIOBLASTOMA INVASION GENE USING LASER CAPTURE MICRODISSECTION,” Cancer Res 61:4190-4196) was detected.

The lack of a similar pattern of gene expression in SCLC may result from the small number of samples examined or may result from different transforming mechanisms since oncogenic mutations (p21^{ras}, p53 and others) but not over-expressions are associated with SCLC (Wistuba, I. I. et al. (2001) “MOLECULAR GENETICS OF SMALL CELL LUNG CARCINOMA,” Semin Oncol 28: 3-13). Functional analysis of genes whose expression significantly altered in pulmonary neuroendocrine tumors provides insight into the underlying biological mechanism, leading to survival and slow progression of TC whereas LCNEC and SCLC have an aggressive behavior.

Many studies have identified genes whose expression is significantly suppressed in neuroendocrine tumors. High incidence of LOH at 3p, 5q, 11q, and 17p (Ohnuki, Y. et al. (1996) “CHROMOSOMAL CHANGES AND PROGRESSIVE TUMORIGENESIS OF HUMAN BRONCHIAL EPITHELIAL CELL LINES,” Cancer Genet. Cytogenet. 92:99-110), except for chromosome 13q, correlates with significant decrease in expression of genes assigned to these locations, including MENI (11q13). The data adds to previously reported studies and confirms that expression profiling of lung neuroendocrine tumors provides accurate tumor classification. The molecular signature of relative abundance of gene expression derived by comparing mean gene expression of each 3 tumor subtypes is independent of the reference RNA and is of particular interest because of its clinical relevance. These results indicate that gene expression profiling of pulmonary neuroendocrine tumors provides a diagnostic tool for tumor classification, particularly when histopathology interpretation is ambiguous.

In summary, light microscopy-based classification of pulmonary neuroendocrine tumors is often difficult. To search for molecular markers of neuroendocrine tumors, cDNA microarrays of 9,984 human transcripts were used to identify classification-associated genes at a global genomic scale. Laser-capture microdissection was used to harvest tumor cells from frozen sections. The gene expression profiles in primary pulmonary neuroendocrine tumors from 17 surgical specimens (11 Typical Carcinoids,(TC), 3 Small Cell lung cancers (SCLC), 2 Large Cell Neuroendocrine tumors (LNEC), and one sample which had features of SCLC and LNEC) were compared. The BRB ArrayTool (National Cancer Institute, NIH; http://linus.nci.nih.gov/BRB-ArrayTools.html) was employed to analyze gene expression patterns. An unsupervised, hierarchical clustering algorithm used to analyze these 17 tumors based only on similarities in gene expression resulted in a precise classification of each tumor type. The Class Comparison Tool used to compare each tumor type identified 198 statistically significant genes (p<0.004) that accurately discriminated between 3 pre-defined tumor types. Analysis of these genes revealed that deletions were more frequent than were amplifications in pulmonary neuroendocrine tumors. Using comparative analysis of gene expression variance, a molecular signature for each tumor type was identified. The signature genes included decreased expression of pro-apoptotic genes, cell-cell and cell matrix interacting components, cell cycle control and DNA repair, and anti-oncogenes. In particular, decreased expression of the BCL2 antagonist, BAK1, was found in all tumor types, whereas BAD was decreased in LCNEC and TC tumors. Over-expression of several growth factors and receptors (CSF2RB, PDGFRB, IL13RA2, and IL6ST (gpI30)) was detected only in LCNEC tumors, and increased expression of IL-8Rβ was shared by TC tumor cells. High expression of a neuronal marker, P311, previously reported to promote invasive phenotype in brain tumors, was detected in LCNEC, and a peptide processing enzyme, Carboxypeptidase E (CPE), was found in TC. The analysis indicates that functional genomic comparison of expression profiles can accurately classify pulmonary neuroendocrine tumors and will therefore facilitate the development of new therapies for patients having these malignancies.

Table 5 lists genes that are differentially expressed in different neuroendocrine tumors.

TABLE 5
Genes Differentially Expressed In Small Cell Lung Cancer (SCLC)
Neuroendocrine Tumor Cells Relative To Large Cell Neuroendocrine
Carcinoma (LCNEC) Neuroendocrine Tumor Cells

| | | |
|---|---|---|
| IncytePD: 523635 | IncytePD: 1734113 | IncytePD: 2074154 |
| IncytePD: 561992 | IncytePD: 1743234 | IncytePD: 2104145 |
| IncytePD: 605019 | IncytePD: 1749727 | IncytePD: 2172334 |
| IncytePD: 614679 | IncytePD: 1755793 | IncytePD: 2180031 |
| IncytePD: 629077 | IncytePD: 1808260 | IncytePD: 2182907 |
| IncytePD: 637639 | IncytePD: 1810821 | IncytePD: 2200079 |
| IncytePD: 696002 | IncytePD: 1821971 | IncytePD: 2205246 |
| IncytePD: 740878 | IncytePD: 1824957 | IncytePD: 2308525 |
| IncytePD: 771715 | IncytePD: 1841920 | IncytePD: 2356635 |
| IncytePD: 820580 | IncytePD: 1853163 | IncytePD: 2374294 |
| IncytePD: 849425 | IncytePD: 1857493 | IncytePD: 2469592 |
| IncytePD: 942207 | IncytePD: 1872067 | IncytePD: 2506427 |
| IncytePD: 958513 | IncytePD: 1890919 | IncytePD: 2507648 |
| IncytePD: 961082 | IncytePD: 1921567 | IncytePD: 2508570 |
| IncytePD: 998069 | IncytePD: 1931265 | IncytePD: 2568547 |
| IncytePD: 1258790 | IncytePD: 1942845 | IncytePD: 2610374 |
| IncytePD: 1297269 | IncytePD: 1960722 | IncytePD: 2663948 |
| IncytePD: 1308112 | IncytePD: 1968721 | IncytePD: 2674277 |
| IncytePD: 1339241 | IncytePD: 1988239 | IncytePD: 3038508 |
| IncytePD: 1382374 | IncytePD: 1990361 | IncytePD: 3115514 |
| IncytePD: 1402615 | IncytePD: 1997937 | IncytePD: 3123858 |
| IncytePD: 1405652 | IncytePD: 1997967 | IncytePD: 3179113 |
| IncytePD: 1431819 | IncytePD: 2048144 | IncytePD: 3202075 |
| IncytePD: 1435374 | IncytePD: 2050085 | IncytePD: 3255437 |
| IncytePD: 1445203 | IncytePD: 2054529 | IncytePD: 3333130 |
| IncytePD: 1453450 | IncytePD: 2055640 | IncytePD: 3360476 |
| IncytePD: 1481225 | IncytePD: 2055687 | IncytePD: 3381870 |
| IncytePD: 1486983 | IncytePD: 2055773 | IncytePD: 3427560 |
| IncytePD: 1501080 | IncytePD: 2055926 | IncytePD: 3432534 |
| IncytePD: 1555545 | IncytePD: 2056149 | IncytePD: 3518380 |
| IncytePD: 1561352 | IncytePD: 2056172 | IncytePD: 3562795 |
| IncytePD: 1567995 | IncytePD: 2056987 | IncytePD: 3842669 |
| IncytePD: 1603584 | IncytePD: 2057547 | IncytePD: 3967780 |
| IncytePD: 1610083 | IncytePD: 2057823 | IncytePD: 3990209 |
| IncytePD: 1624024 | IncytePD: 2058537 | IncytePD: 3999291 |
| IncytePD: 1625169 | IncytePD: 2060308 | IncytePD: 4014715 |
| IncytePD: 1635008 | IncytePD: 2679117 | IncytePD: 4016254 |
| IncytePD: 1637517 | IncytePD: 2740235 | IncytePD: 4059193 |
| IncytePD: 1653911 | IncytePD: 2751387 | IncytePD: 4144001 |
| IncytePD: 1685342 | IncytePD: 2852403 | IncytePD: 4287342 |
| IncytePD: 1691161 | IncytePD: 2956581 | IncytePD: 4626895 |
| IncytePD: 1699149 | IncytePD: 2956906 | IncytePD: 5017148 |
| IncytePD: 1702266 | IncytePD: 3032691 | IncytePD: 5096975 |
| IncytePD: 1969563 | IncytePD: 3032825 | |

Genes Differentially Expressed In Small Cell Lung Cancer (SCLC)
Neuroendocrine Tumor Cells Relative To Typical Carcinoid (TC)
Neuroendocrine Tumor Cells

| | | |
|---|---|---|
| IncytePD: 477045 | IncytePD: 1748705 | IncytePD: 2453436 |
| IncytePD: 478960 | IncytePD: 1749727 | IncytePD: 2469592 |
| IncytePD: 523635 | IncytePD: 1755793 | IncytePD: 2506427 |
| IncytePD: 557451 | IncytePD: 1773638 | IncytePD: 2508570 |
| IncytePD: 561992 | IncytePD: 1807294 | IncytePD: 2610374 |
| IncytePD: 588157 | IncytePD: 1808260 | IncytePD: 2622566 |
| IncytePD: 605019 | IncytePD: 1810821 | IncytePD: 2663948 |
| IncytePD: 696002 | IncytePD: 1812955 | IncytePD: 2674277 |
| IncytePD: 740878 | IncytePD: 1822716 | IncytePD: 2679117 |
| IncytePD: 771715 | IncytePD: 1824957 | IncytePD: 2722572 |
| IncytePD: 818568 | IncytePD: 1841920 | IncytePD: 2728840 |
| IncytePD: 820580 | IncytePD: 1853163 | IncytePD: 2740235 |
| IncytePD: 885601 | IncytePD: 1857493 | IncytePD: 2748942 |
| IncytePD: 899102 | IncytePD: 1858365 | IncytePD: 2751387 |
| IncytePD: 958513 | IncytePD: 1872067 | IncytePD: 2758740 |
| IncytePD: 961082 | IncytePD: 1890919 | IncytePD: 2798872 |
| IncytePD: 1240748 | IncytePD: 1920650 | IncytePD: 2806778 |
| IncytePD: 1258790 | IncytePD: 1921567 | IncytePD: 2852403 |
| IncytePD: 1297269 | IncytePD: 1931265 | IncytePD: 2888814 |
| IncytePD: 1308112 | IncytePD: 1942845 | IncytePD: 2914719 |
| IncytePD: 1402615 | IncytePD: 1960722 | IncytePD: 2923082 |
| IncytePD: 1405652 | IncytePD: 1968721 | IncytePD: 2956906 |
| IncytePD: 1431819 | IncytePD: 1988239 | IncytePD: 3010959 |
| IncytePD: 1435374 | IncytePD: 1997792 | IncytePD: 3032691 |
| IncytePD: 1445203 | IncytePD: 2050085 | IncytePD: 3032825 |
| IncytePD: 1453450 | IncytePD: 2054529 | IncytePD: 3038508 |
| IncytePD: 1481225 | IncytePD: 2055640 | IncytePD: 3115514 |
| IncytePD: 1486983 | IncytePD: 2055687 | IncytePD: 3123858 |
| IncytePD: 1488021 | IncytePD: 2055773 | IncytePD: 3179113 |
| IncytePD: 1505977 | IncytePD: 2055926 | IncytePD: 3202075 |
| IncytePD: 1513989 | IncytePD: 2056149 | IncytePD: 3334367 |
| IncytePD: 1559756 | IncytePD: 2056172 | IncytePD: 3381870 |
| IncytePD: 1561867 | IncytePD: 2056642 | IncytePD: 3432534 |
| IncytePD: 1562658 | IncytePD: 2056987 | IncytePD: 3518380 |
| IncytePD: 1567995 | IncytePD: 2057547 | IncytePD: 3562795 |
| IncytePD: 1603584 | IncytePD: 2057823 | IncytePD: 3728255 |
| IncytePD: 1610083 | IncytePD: 2057908 | IncytePD: 3805046 |
| IncytePD: 1624024 | IncytePD: 2058537 | IncytePD: 3871545 |
| IncytePD: 1625169 | IncytePD: 2060308 | IncytePD: 3954785 |
| IncytePD: 1635008 | IncytePD: 2074154 | IncytePD: 3967780 |
| IncytePD: 1653911 | IncytePD: 2104145 | IncytePD: 3990209 |
| IncytePD: 1669254 | IncytePD: 2153373 | IncytePD: 3999291 |
| IncytePD: 1672749 | IncytePD: 2172334 | IncytePD: 4014715 |
| IncytePD: 1691161 | IncytePD: 2180031 | IncytePD: 4059193 |
| IncytePD: 1693847 | IncytePD: 2182907 | IncytePD: 4144001 |
| IncytePD: 1699149 | IncytePD: 2304121 | IncytePD: 4253663 |
| IncytePD: 1702266 | IncytePD: 2356635 | IncytePD: 4626895 |
| IncytePD: 1704168 | IncytePD: 2369544 | IncytePD: 5017148 |
| IncytePD: 1712663 | IncytePD: 2374294 | IncytePD: 5096975 |
| IncytePD: 1734113 | IncytePD: 2383065 | |

Genes Differentially Expressed In Large Cell Neuroendocrine
Carcinoma (LCNEC) Neuroendocrine Tumor Cells Relative To
Typical Carcinoid (TC) Neuroendocrine Tumor Cells

| | | |
|---|---|---|
| IncytePD: 629077 | IncytePD: 1748705 | IncytePD: 2507648 |
| IncytePD: 637639 | IncytePD: 1773638 | IncytePD: 2508570 |
| IncytePD: 818568 | IncytePD: 1807294 | IncytePD: 2622566 |
| IncytePD: 885601 | IncytePD: 1812955 | IncytePD: 2679117 |
| IncytePD: 899102 | IncytePD: 1821971 | IncytePD: 2728840 |
| IncytePD: 942207 | IncytePD: 1822716 | IncytePD: 2806778 |
| IncytePD: 1308112 | IncytePD: 1858365 | IncytePD: 2888814 |
| IncytePD: 1402615 | IncytePD: 1872067 | IncytePD: 2914719 |
| IncytePD: 1435374 | IncytePD: 1990361 | IncytePD: 2956581 |
| IncytePD: 1488021 | IncytePD: 1997967 | IncytePD: 3255437 |
| IncytePD: 1501080 | IncytePD: 2048144 | IncytePD: 3333130 |
| IncytePD: 1505977 | IncytePD: 2153373 | IncytePD: 3360476 |
| IncytePD: 1555545 | IncytepD: 2205246 | IncytePD: 3427560 |
| IncytePD: 1559756 | IncytePD: 2299818 | IncytePD: 3518380 |
| IncytePD: 1561352 | IncytePD: 2304121 | IncytePD: 3805046 |
| IncytePD: 1561867 | IncytePD: 2308525 | IncytePD: 4016254 |
| IncytePD: 1610993 | IncytePD: 2369544 | IncytePD: 4144001 |
| IncytePD: 1704168 | IncytePD: 2453436 | IncytePD: 4287342 |
| IncytePD: 1712663 | IncytePD: 2469592 | |
| IncytePD: 1743234 | IncytePD: 2506427 | |

The methods employed in the present invention can be similarly employed to facilitate the diagnosis of other tumor types, for example, adenocarcinomas, which are distinct from neuroendocrine tumors and exhibit significant differences in gene expression (Garber, M. E. et al. (2001) “DIVERSITY OF GENE EXPRESSION IN ADENOCARCINOMA OF THE LUNG” Proc. Natl. Acad. Sci. (U.S.A.) 98:13784-13789; Bhattacharjee, A. et al. (2001) “CLASSIFICATION OF HUMAN LUNG CARCINOMAS BY mRNA EXPRESSION PROFILING REVEALS DISTINCT ADENOCARCINOMA SUBCLASSES” Proc. Natl. Acad. Sci. (U.S.A.) 98:13790-13795). cDNA microarrays that can be used to identify profiles of genes expressed in adenocarcinomas are disclosed by Miura, K. et al. (2002) (“LASER CAPTURE MICRODISSECTION AND MICROARRAY EXPRESSION ANALYSIS OF LUNG ADENOCARCINOMA REVEALS TOBACCO SMOKING-AND PROGNOSIS-RELATED MOLECULAR PROFILES,” Canc. Res. 62:3244-3250).

### EXAMPLE 4

### Analysis of Gene Expression Profiles

As indicated above, DNA microarray technology (Schena, M. et al. (1995) “QUANTITATIVE MONITORING OF GENE EXPRESSION PATTERNS WITH A COMPLEMENTARY DNA MICROARRAY,” Science 270:467-470; DeRisi, J. et al. (1996) “USE OF A cDNA MICROARRAY TO ANALYSE GENE EXPRESSION PATTERNS IN HUMAN CANCER,” Nat Genet 14:457-460) provides a powerful tool to analyze genome-wide changes in gene expression. Applications of this technology to human lung cancers facilitate the identification of gene expression profiles and biomarkers associated with adenocarcinoma (Miura, K. et al. (2002) “Laser capture microdissection and microarray expression analysis of lung adenocarcinoma reveals tobacco smoking- and prognosis-related molecular profiles,” Cancer Res 62:3244-3250; Sugita, M. et al., (2002) “COMBINED USE OF OLIGONUCLEOTIDE AND TISSUE MICROARRAYS IDENTIFIES CANCER/TESTIS ANTIGENS AS BIOMARKERS IN LUNG CARCINOMA,” Cancer Res 62:3971-3979; Bhattacharjee, A. et al. (2001) “CLASSIFICATION OF HUMAN LUNG CARCINOMAS BY mRNA EXPRESSION PROFILING REVEALS DISTINCT ADENOCARCINOMA SUBCLASSES,” Proc Natl Acad Sci USA 2001; 98:13790-13795) and NSCLC (Heighway, J. et al. (2002) “EXPRESSION PROFILING OF PRIMARY NON-SMALL CELL LUNG CANCER FOR TARGET IDENTIFICATION,” Oncogene 2002; 21:7749-7763; Kikuchi, T. et al. (2003) “EXPRESSION PROFILES OF NON-SMALL CELL LUNG CANCERS ON cDNA MICROARRAYS: IDENTIFICATION OF GENES FOR PREDICTION OFLYMPH-NODE METASTASIS AND SENSITIVITY TO ANTI-CANCER DRUGS,” Oncogene 22:2192-2205). These studies lead to the identification of molecular markers with a potential for better diagnosis, more accurate prediction of prognosis, and selection of effective treatment modalities.

To identify expression profiles and biomarkers for pulmonary NET, laser capture microdissection (LCM) (Emmert-Buck, M. R. et al. (1996) “LASER CAPTURE MICRODISSECTION” Science 1996; 274:9981001; Bonner, R. F. et al. (1997) “LASER CAPTURE MICRODISSECTION: MOLECULAR ANALYSIS OF TISSUE,” Science 278:1481,1483) and cDNA microarrays (Schena, M. et al. (1995) “QUANTITATIVE MONITORING OF GENE EXPRESSION PATTERNS WITH A COMPLEMENTARY DNA MICROARRAY,” Science 270:467-470; DeRisi, J. et al. (1996) “USE OF A cDNA MICROARRAY TO ANALYSE GENE EXPRESSION PATTERNS IN HUMAN CANCER,” Nat Genet 14:457-460) on 17 cases of primary pulmonary NET including TC (n=11), LCNEC (n=2), SCLC (n=3) and one case of LCNEC combined with SCLC are conducted. The resultant clustering of expression profiles corresponding to the subtype pulmonary NET are verified by real-time RT-PCR analysis and matched completely with the histological classification. Of 48 classifier genes identified, two are subjected to protein expression analysis by in situ immunohistochemistry (IHC) on 55 pulmonary NET cases, which result in the identification of carboxypeptidase E (CPE) and γ-glutamyl hydrolase (GGH) as diagnostic biomarkers to differentiate low- and intermediate-grades TC and AC from high-grade LCNEC and SCLC. Kaplan-Meier survival analysis reveals that the protein expressions of these two biomarkers can serve as prognosis indicators for pulmonary NET patients.

Materials and Methods

Tissue samples. Fresh frozen tissues of 17 primary pulmonary NET were collected from hospitals over an 11-year period. Tissues were flash-frozen after surgery and stored at −80° C. until used. Histopathological classification of these tumors was based on the 1999 WHO/LASLSC classification of “Histological Typing of Lung and Pleural Tumors” (see, Travis, W. D. et al. (1998) “REPRODUCIBILITY OF NEUROENDOCRINE LUNG TUMOR CLASSIFICATION,” Hum Pathol. 29:272-279). The tissues were used for microarray and IHC. A total of 68 cases (29 TCs, five ACs, nine LCNECs, and 25 SCLCs) were used for IHC and 55 cases generated informative data. Fifty-four of 55 cases have clinical survival data and are used for Kaplan-Meier survival analysis.

Laser capture microdissection. Frozen tissue (0.5×0.5×0.5 cm) is embedded in OCT in a cryomold, and immersed immediately in dry ice-cold 2-methylbutane at −50° C. Tissue sections (8 μm) are mounted on Silane-coated slides and kept at −80° C. until use. The slides are fixed by immersion in 70% ethanol, stained with H&E and air-dried for 10 min after xylene treatment.

The PixCell™ LCM system was used for LCM (Emmert-Buck, M. R. et al. (1996) “LASER CAPTURE MICRODISSECTION” Science 1996; 274:9981001; Bonner, R. F. et al. (1997) “LASER CAPTURE MICRODISSECTION: MOLECULAR ANALYSIS OF TISSUE,” Science 278:1481,1483). Tumor cells are fused to transfer film by thermal adhesion after laser pulse and transferred into tubes for RNA extraction. Total RNA is extracted using Micro RNA isolation kit (Strategene, La Jolla, Calif.) according to the manufacturer's instructions. RNA quality is evaluated by spectrophotometry and gel electrophoresis. Purified RNA is dissolved into 11 μl of DEPC-treated water and used for amplification. The amplified RNA is subjected to cDNA microarray analysis (Schena, M. et al. (1995) “QUANTITATIVE MONITORING OF GENE EXPRESSION PATTERNS WITH A COMPLEMENTARY DNA MICROARRAY,” Science 270:467-470; DeRisi, J. et al (1996) “USE OF A cDNA MICROARRAY TO ANALYSE GENE EXPRESSION PATTERNS IN HUMAN CANCER,” Nat Genet 14:457-460).

Tissue Culture. A cell line derived from normal bronchial epithelium, BEAS-2B, is cultured in a serum-free medium, LHC-9, and harvested at passage 30. Total RNA is isolated from cultured cells using Micro RNA isolation kit (Strategene) according to the manufacturer's instructions.

RNA amplification. RNA amplification was performed as described by Luo, L. et al. (1999) (“GENE EXPRESSION PROFILES OF LASER-CAPTURED ADJACENT NEURONAL SUBTYPES,” Nat Med 1999; 5:117-122). Briefly, oligo (dT) primers with T7 promoter sequence (SEQ ID NO: 1) is used to synthesize the first strand of cDNA. After the second strand of cDNA synthesis, RNA is amplified by using T7 RNA polymerase on the cDNA templates. Two rounds of amplification starting with 1 μg of total RNA generate 40-60 μg of amplified RNA, which is used for microarray analysis.

Microarray, Hybridization, and Analysis. cDNA microarrays with 9,984 human genes per slide are provided by the Advanced Technology Center (National Cancer Institute, Bethesda, MD). Six of 17 samples are hybridized with two slides to work out microarray labeling and hybridization procedures for consensus expression data (>95% Pearson Coefficient Correlation between two slides hybridized with the same samples). The remaining samples are conducted under the same labeling and hybridization conditions. RNA (8 μg), amplified from the BEAS-2B cell line (passage 30), is labeled with Cy5-dUTP as a reference. Amplified RNA (4 μg each) from tumors is labeled with Cy3-dUTP by using Superscript II (Invitrogen, Carlsbad, Calif.). Briefly, RNA is incubated with Cy3-dUTP (or Cy5-dUTP) (Perkin Elmer Life Sciences, Boston, Mass.) at 42° C. for 1 h to synthesize the first strand cDNA. The reaction is stopped by the addition of 5 μl 0.5M EDTA and the RNA is degraded by the addition of 10 μl 1N NaOH and then incubation at 65° C. for 60 min. After neutralizing, the samples are purified by Microcon 30 (Millipore Corp., Bedford, Mass.). Each pair of labeled samples is hybridized to DNA on slides at 65° C. for 16 h. After washing, the slides are scanned with a GenePix 4000A scanner (Axon Instruments, Inc., Foster City, Calif.). Hierarchical clustering and gene selection are performed by using BRB-ArrayTools V 3.0 (National Cancer Institute, Bethesda Md., http://linus.nci.nih.gov/brb).

Real-time PCR. Total RNA is purified from LCM cells, using the Stratagene Absolutely RNA™ microprep kit. Samples are treated by DNase I to eliminate DNA contamination. Primers are designed, using Primer Express Software V 1.5 (Applied Biosystmes Inc., Foster City, Calif.) based on sequences from GenBank and purchased from Biosource International (Camarillo, Calif.). Final probe concentration was 200 nM for each gene. Endogenous 1 8s RNA (Applied Biosystems) is used as an internal reference. Reverse transcription is completed with the RT-EZ RNA kit (Applied Biosystems) according to the manufacturer's instructions. Samples are run in triplicate and monitored on the ABI PRISM 7700.

Immunohistochemistry. Immunohistochemistry is performed by the avidin-biotin peroxidase complex (ABC) method (Vectastain Elite ABC kit, Vector, Calif.). Briefly, slides are deparaffinized, and rehydrated through xylene and alcohol in Coplin jars. Endogenous peroxidase is blocked with 3% H₂O₂ in phosphate-buffered saline (PBS) for 20 min. All washes are in PBS at room temperature if not mentioned. After two washes, Heat Induced Epitope Retrieval (HIER) is performed in a citrate buffer (pH: 6.0) in a Biocare Medical chamber (Walnut Creek, Calif.). Slides are rinsed, enclosed with a PAP pen, placed in the humid chamber, and incubated first with Protein Block (normal GOAT serum diluted in PBS containing 1% BSA, 0.09% sodium azide, 0.1% Tween-20 [BioGenex, CA]), and then with primary antibody: GGH (rabbit polyclonal, Dr. Thomas J. Ryan, Wadsworth Center, N.Y. State Dept. of Health, Albany, N.Y., 1:1000 diluted by Universal blocking reagent [BioGenex]) and CPE (rabbit polyclonal, Dr. Lloyd Fricker, Albert Einstein College of Medicine, NY, 1:500 dilution) for 1 h. After three washes, slides are incubated for 30 min with biotinylated goat anti rabbit IgG (Vector, 1:250 dilution). After three washes, the slides are incubated for 45 min with the ABC reagent (Vector). Slides are washed twice, placed in Tris-HCl buffer (pH 7.5) for 5 min, developed with liquid DAB (DAKO, Calif.) for 3 min, washed with H₂O twice, and finally counterstained lightly with Mayer's hematoxyline for 5 sec, dehydrated, cleared, and mounted with resinous mounting medium. Signal intensity and distribution are based on the publication (Gillett, C. et al. (1994) “AMPLIFICATION AND OVEREXPRESSION OF CYCLIN D1 IN BREAST CANCER DETECTED BY IMMUNOHISTOCHEMICAL STAINING,” Cancer Res 54: 1812-1817; Beasley, M. B. et al. (2003) “The P16/CYCLIN D1/RB PATHWAY IN NEUROENDOCRINE TUMORS OF THE LUNG,” Hum Pathol. 34:136-142) and scored blindly by three pathologists as follows: distribution score (DS) is graded as 0, absent; 1, <10%; 2, 10% to 50%; 3, 51% to 90%; or 4, >90%. Intensity score (IS) is graded as ISO, no signal; IS1, weak; IS2, medium; or IS3, strong. The combined total score is determined as total score (TS)=distribution (DS)+intensity (IS) (TS0, sum 0; TS1, sum 1 to 3; TS2, sum 4 to 5; TS3, sum 6 to 7). TS0 and TS 1 are considered negative, whereas TS2 and TS3 are considered positive, respectively.

Statistics. Binomial distributions are used to compute p-values between positive and negative immunohistochemical stains of anti-CPE or anti-GGH antibodies to tissue sections. Kaplan-Meier survival is calculated in the statistic software SPSS 9.0 for Windows. A p-value less than 0.05 or 0.01 is used as significant or very significant statistical indicator, respectively.

Results

Microarray Analysis and Expression Classification of Pulmonary NET.

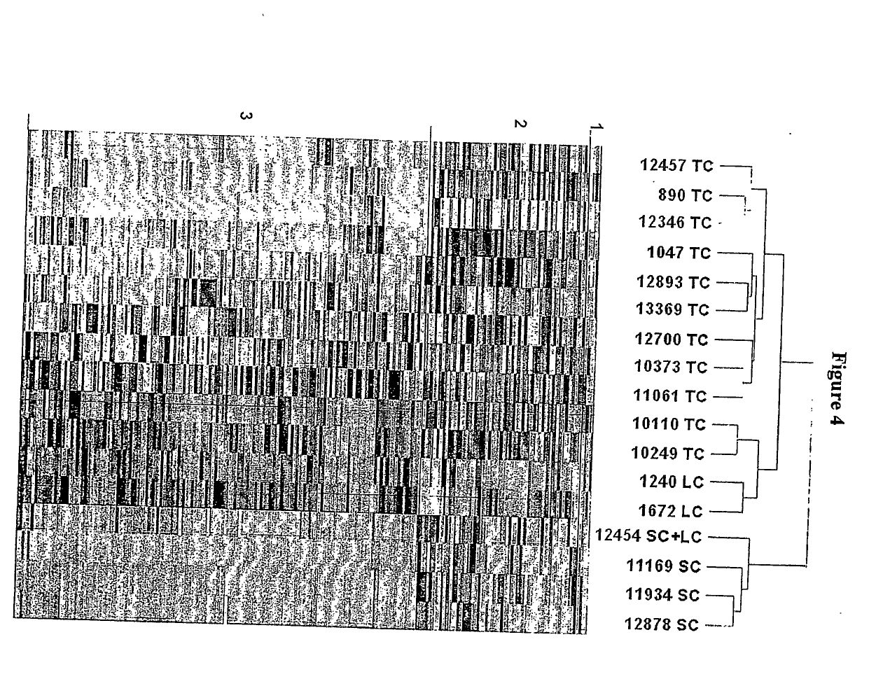

Homogeneous cancer cells are collected from pulmonary NET tissue sections by LCM avoiding contamination with other cells to conduct microarray analysis of gene expression. LCM is performed on 15-18 frozen sections per sample to maximize the number of homogeneous cells from each of 17 available fresh frozen pulmary NET (11 TC, two LCNEC, three SCLC, and one combined SCLC and LCNEC). High quality total RNA (>1 μg/sample) is purified from the dissected cells and subjected to two rounds of RNA amplification by T7 RNA polymerase (Luo, L. et al. (1999) “GENE EXPRESSION PROFILES OF LASER-CAPTURED ADJACENT NEURONAL SUBTYPES” Nature Medicine 5:117-2216) for microarray analasis. cDNA microarrays of 9,984 genes are hybridized by Cy3-labeled cDNA from 4 μg tumor RNA and Cy5-labeled reference cDNA from 8 μg RNA of the normal bronchial epithelial cell line BEAS-2B (Reddel, R. R. et al. (1988) “TRANSFORMATION OF HUMAN BRONCHIAL EPITHELIAL CELLS BY INFECTION WITH SV40 OR ADENOVIRUS-12 SV40 HYBRID VIRUS, OR TRANSFECTION VIA STRONTIUM PHOSPHATE CCOPRECIPITATION WITH A PLASMID CONTAINING SV 40 EARLY REGION GENES,” Cancer Res 48:1904-1909) for all 17 samples. Hierarachical clustering analysis on expression levels of 9,984 genes without prior knowledge of sample identity reveals the sample clusters matching histological classification. An F-test is then conducted by use of the BRB array tool to measure variance in gene expression in each sample among three defined subtypes. Based on arbitrary criteria of 2-fold changes and p-value <0.004, 198 genes are identified (Table 6) that also clustered the 17 tumors into groups in agreement with the morphological classification (FIG. 4).

TABLE 6
Cluster Genes, Using Average Linkage and EuclideanDistance,
and Cutting Tree at Three Clusters

| | | | | | |
|---|---|---|---|---|---|
| | Unique | | | | |
| No. | ID | Gene Symbol | Map | Clone Incyte PD No. | UG Cluster |

Cluster #1

| | | | | | |
|---|---|---|---|---|---|
| 1 | 166807 | GRIA2 | 4q32-q33 | 1505977 | Hs.89582 |
| 2 | 159877 | CPE | 4q32.3 | 2153373 | Hs.75360 |
| 3 | 161598 | ORC4L | 2q22-q23 | 2728840 | Hs.55055 |
| 4 | 167158 | C5 | 9q32-q34 | 1712663 | Hs.1281 |

Cluster #2

| | | | | | |
|---|---|---|---|---|---|
| 5 | 167153 | GGH | 8q12.1 | 1997967 | Hs.78619 |
| 6 | 160605 | P311 | 5q21.3 | 1555545 | Hs.142827 |
| 7 | 169429 | NR3C1 | 5q31 | 629077 | Hs.75772 |
| 8 | 165192 | SYNJ2 | 6q25-26 | 3954785 | Hs.61289 |
| 9 | 165784 | ADD3 | 10q24.2-q24.3 | 1481225 | Hs.324470 |
| 10 | 163031 | KIM0751 | 8q23.1 | 2369544 | Hs.153610 |
| 11 | 166328 | PSMC6 | 12q15 | 1488021 | Hs.79357 |
| 12 | 168061 | FTHFD | 3q21.3 | 2104145 | Hs.9520 |
| 13 | 168141 | DGKG | 3q27-q28 | 2568547 | Hs.89462 |
| 14 | 165076 | SMG1 | 16p12.3 | 4253663 | Hs.110613 |
| 15 | 167103 | TAF2 | 8q24.12 | 998069 | Hs.122752 |
| 16 | 169391 | EIF2S1 | 14q23.3 | 1224219 | Hs.151777 |
| 17 | 166789 | ZNF202 | 11q23.3 | 1997937 | Hs.9443 |
| 18 | 167316 | SLC24A1 | 15q22 | 2200079 | Hs.173092 |
| 19 | 168700 | FPRL 1 | 19q13.3-q13.4 | 523635 | Hs.99855 |
| 20 | 165576 | IL6ST | 5q11 | 2172334 | Hs.82065 |
| 21 | 168276 | ITGBL 1 | 13q33 | 1258790 | Hs.82582 |
| 22 | 169180 | IL8RB | 2q35 | 561992 | Hs.846 |
| 23 | 160957 | PRKM2 | 1p31 | 2507648 | Hs.2329 |
| 24 | 160617 | CSF2RB | 22q13.1 | 1561352 | Hs.285401 |
| 25 | 160429 | PTK6 | 20q13.3 | 3255437 | Hs.51133 |
| 26 | 160237 | NPAT | 11q22-q23 | 2308525 | Hs.89385 |
| 27 | 167125 | TNFRSF6 | 10q24.1 | 2205246 | Hs.82359 |
| 28 | 164652 | PDGFRB | 5q31-q32 | 1821971 | Hs.76144 |
| 29 | 161117 | ABCG2 | 4q22 | 1501080 | Hs.194720 |
| 30 | 161896 | COL 15A1 | 9q21-q22 | 4287342 | Hs.83164 |
| 31 | 159813 | PTPN12 | 7q11.23 | 1382374 | Hs.62 |
| 32 | 164573 | DMTF1 | 7q21 | 1637517 | Hs.5671 |
| 33 | 169384 | SLC22A1LS | 11p15.5 | 3842669 | Hs.300076 |
| 34 | 165393 | | | 3202075 | Hs.351699 |
| 35 | 168169 | OXCT | 5p13 | 1685342 | Hs.177584 |
| 36 | 165617 | PRLR | 5p14-p13 | 3427560 | Hs.1906 |
| 37 | 169432 | IL 13RA2 | Xq13.1-q28 | 3360476 | Hs.25954 |
| 38 | 166812 | MPZL 1 | 1q23.2 | 2057323 | Hs.287832 |
| 39 | 168428 | RU NX3 | 1p36 | 885297 | Hs.170019 |
| 40 | 167180 | S100A4 | 1q21 | 1222317 | Hs.81256 |
| 41 | 161533 | CSTF2 | Xq21.33 | 4016254 | Hs.693 |
| 42 | 165588 | SNAPC4 | 9q34.3 | 2224902 | Hs.113265 |
| 43 | 164799 | EMP3 | 19q13.3 | 780992 | Hs.9999 |
| 44 | 161709 | FLJ11560 | 9p12 | 1990361 | Hs.301696 |
| 45 | 164868 | GBP2 | 1pter-p13.2 | 1610993 | Hs.171862 |
| 46 | 160233 | DYRK3 | 1q32 | 614679 | Hs.38018 |
| 47 | 165400 | MY040 | 7q35-q36 | 2048144 | Hs.124854 |
| 48 | 165957 | PNLIPRP2 | 10q26.12 | 885032 | Hs.143113 |
| 49 | 160054 | SEC4L | 17q25.3 | 1824556 | Hs.302498 |
| 50 | 162475 | CTAG2 | Xq28 | 849425 | Hs.87225 |
| 51 | 169182 | LOC56311 | 7q31 | 2013272 | Hs.73073 |
| 52 | 162912 | DKFZP566B084 | 3q13 | 2680168 | Hs.21201 |
| 53 | 163475 | FLJ20485 | 7q22.1 | 2299818 | Hs.98806 |
| 54 | 164927 | HNRPAO | 5q31 | 637639 | Hs.77492 |
| 55 | 160630 | HOXD9 | 2q31-q37 | 2956581 | Hs.236646 |
| 56 | 160367 | JUN | 1p32-p31 | 1969563 | Hs.78465 |
| 57 | 163762 | | 17 | 1743234 | Hs.120854 |
| 58 | 162247 | VLGR1 | 5q13 | 942207 | Hs.153692 |
| 59 | 167219 | PUM1 | 1p35.2 | 3333130 | Hs.153834 |

Cluster #3

| | | | | | |
|---|---|---|---|---|---|
| 60 | 165171 | KRT18 | 12q13 | 1435374 | Hs.65114 |
| 61 | 165052 | CDC20 | 9q13-q21 | 2469592 | Hs.82906 |
| 62 | 167948 | PIM1 | 6p21.2 | 2679117 | Hs.81170 |
| 63 | 161954 | ATP6F | 1p32.3 | 5017148 | Hs.7476 |
| 64 | 162391 | POLE3 | 9q33 | 961082 | Hs.108112 |
| 65 | 166635 | KRT5 | 12q12-q13 | 3432534 | Hs.195850 |
| 66 | 160035 | FEN1 | 11q12 | 2050085 | Hs.4756 |
| 67 | 161774 | SIP2-28 | 15q25.3-q26 | 4626895 | Hs.10803 |
| 68 | 162207 | VATI | 17q21 | 2060308 | Hs.157236 |
| 69 | 161163 | GUK1 | 1q32-q41 | 2506427 | Hs.3764 |
| 70 | 161223 | SIVA | 22 | 2356635 | Hs.112058 |
| 71 | 161211 | CAPG | 2cen-q24 | 2508570 | Hs.82422 |
| 72 | 161948 | CLDN11 | 3q26.2-q26.3 | 4144001 | Hs.31595 |
| 73 | 161391 | IL17F | 6p12 | 1610083 | Hs.272295 |
| 74 | 162571 | PFKL | 21q22.3 | 885601 | Hs.155455 |
| 75 | 164504 | CTSC | 11q14.1-q14.3 | 1822716 | Hs.10029 |
| 76 | 160565 | ACY1 | 3p21.1 | 1812955 | Hs.334707 |
| 77 | 169551 | GSK3B | 3q13.3 | 2057908 | Hs.78802 |
| 78 | 166914 | METTL 1 | 12q13 | 1603584 | Hs.42957 |
| 79 | 167738 | CYP27B 1 | 12q13.1-q13.3 | 1749727 | Hs.199270 |
| 80 | 160938 | HMGE | 4p16 | 2074154 | Hs.151903 |
| 81 | 162734 | WNT7 A | 3p25 | 2622566 | Hs.72290 |
| 82 | 165813 | CASP4 | 11q22.2-q22.3 | 2304121 | Hs.74122 |
| 83 | 159898 | PTTG1 | 5q35.1 | 1748705 | Hs.252587 |
| 84 | 161244 | ARF4L | 17q12-q21 | 2852403 | Hs.183153 |
| 85 | 160715 | CDC34 | 19p13.3 | 1857493 | Hs.76932 |
| 86 | 163787 | PYCR1 | 17q24 | 1702266 | Hs.79217 |
| 87 | 160127 | PGAM1 | 10q25.3 | 3032691 | Hs.181013 |
| 88 | 160323 | ATIC | 2q35 | 2056149 | Hs.90280 |
| 89 | 164850 | IRAK1 | Xq28 | 1872067 | Hs.182018 |
| 90 | 165583 | DHCR7 | 11q13.2-q13.5 | 3518380 | Hs.11806 |
| 91 | 165039 | TK1 | 17q23.2-q25.3 | 2055926 | Hs.105097 |
| 92 | 167964 | CDKN2A | 9p21 | 2740235 | Hs.1174 |
| 93 | 167223 | GNB1 | 1p36.21-36.33 | 3562795 | Hs.215595 |
| 94 | 167931 | CSTF1 | 20q13.2 | 1635008 | Hs.172865 |
| 95 | 163690 | HXB | 9q33 | 1453450 | Hs.289114 |
| 96 | 161955 | CNTN2 | 1q32.1 | 4014715 | Hs.2998 |
| 97 | 160275 | SSRP1 | 11q12 | 2055773 | Hs.79162 |
| 98 | 168110 | TAF12 | 1p35.1 | 1297269 | Hs.82037 |
| 99 | 160102 | ERP70 | 10 | 1824957 | Hs.93659 |
| 100 | 167116 | NP | 14q13.1 | 2453436 | Hs.75514 |
| 101 | 160802 | PHB | 17q21 | 1625169 | Hs.75323 |
| 102 | 161643 | ARL7 | 2q37.2 | 3115514 | Hs.111554 |
| 103 | 162343 | LIMK2 | 22q12.2 | 958513 | Hs.278027 |
| 104 | 162727 | PTK9L | 3p21.1 | 3999291 | Hs.6780 |
| 105 | 160262 | DDX28 | 16q22.1 | 2663948 | Hs.155049 |
| 106 | 165790 | SURF1 | 9q33-q34 | 1921567 | Hs.3196 |
| 107 | 168638 | HDAC7A | 12q13.1 | 1968721 | Hs.275438 |
| 108 | 168079 | EMP1 | 12p12.3 | 1624024 | Hs.79368 |
| 109 | 160999 | P114-RHO-GEF | 19p13.3 | 1734113 | Hs.6150 |
| 110 | 161790 | KIM0469 | 1p36.23 | 2674277 | Hs.7764 |
| 111 | 169691 | E2-EPF | 17p12-p11 | 2057823 | Hs.174070 |
| 112 | 163682 | DPH2L2 | 1p34 | 1810821 | Hs.324830 |
| 113 | 168266 | PSME3 | 17q12-q21 | 1308112 | Hs.152978 |
| 114 | 161374 | POLA2 | 11q13.1 | 3179113 | Hs.81942 |
| 115 | 164646 | GALE | 1p36-p35 | 1807294 | Hs.76057 |
| 116 | 162150 | APOL 1 | 22q13.1 | 2056987 | Hs.114309 |
| 117 | 164206 | FN14 | 16p13.3 | 1402615 | Hs.10086 |
| 118 | 162623 | BAK1 | 6p21.3 | 2055687 | Hs.93213 |
| 119 | 162244 | ARHGDIA | 17q25.3 | 2055640 | Hs.159161 |
| 120 | 164586 | ITPA | 20p | 1931265 | Hs.6817 |
| 121 | 165483 | PDAP1 | 7q22.1 | 3032825 | Hs.278426 |
| 122 | 166195 | APRT | 16q24 | 2751387 | Hs.28914 |
| 123 | 166960 | APG12L | 5q21-q22 | 2058537 | Hs.264482 |
| 124 | 167505 | TST | 22q13.1 | 1988239 | Hs.351863 |
| 125 | 168642 | ST14 | 11q24-q25 | 478960 | Hs.56937 |
| 126 | 167170 | DXS1283E | Xp22.3 | 1567995 | Hs.264 |
| 127 | 161754 | ACTG2 | 2p13.1 | 3381870 | Hs.78045 |
| 128 | 166010 | RIPK1 | 6p25.3 | 2180031 | Hs.296327 |
| 129 | 161794 | SCAMP2 | 15q23-q25 | 3123858 | Hs.238030 |
| 130 | 167591 | COMT | 22q11.21 | 605019 | Hs.240013 |
| 131 | 162587 | POLR2D | 2q21 | 696002 | Hs.194638 |
| 132 | 169071 | CAPZB | 1p36.1 | 1853163 | Hs.333417 |
| 133 | 160467 | POLD2 | 7p13 | 2056172 | Hs.74598 |
| 134 | 162178 | C2F | 12p13 | 5096975 | Hs.12045 |
| 135 | 167706 | GMPPB | 3p21.31 | 1486983 | Hs.28077 |
| 136 | 160803 | FARSL | 19p13.2 | 1808260 | Hs.23111 |
| 137 | 169254 | POLM | 7p13 | 771715 | Hs.46964 |
| 138 | 167351 | MYBPH | 1q32.1 | 3010959 | Hs.927 |
| 139 | 163276 | | 7 | 2383065 | Hs.25892 |
| 140 | 167135 | ERCC1 | 19q13.2-q13.3 | 2054529 | Hs.59544 |
| 141 | 160478 | G5B | 6p21.3 | 1942845 | Hs.73527 |
| 142 | 162631 | T ADA3L | 3p25.2 | 3990209 | Hs.158196 |
| 143 | 163921 | GNPI | 5q21 | 1653911 | Hs.278500 |
| 144 | 160098 | MRPL49 | 11q13 | 1755793 | Hs.75859 |
| 145 | 161058 | MEN1 | 11q13 | 1693847 | Hs.24297 |
| 146 | 160038 | BAD | 11q13.1 | 3967780 | Hs.76366 |
| 147 | 162220 | FKBP1A | 20p13 | 4059193 | Hs.349972 |
| 148 | 161026 | HSXQ280RF | Xq28 | 1669254 | Hs.6487 |
| 149 | 167607 | TRAP1 | 16p13.3 | 1960722 | Hs.182366 |
| 150 | 167713 | KIM0175 | 9p11.2 | 3805046 | Hs.184339 |
| 151 | 165648 | DUSP4 | 8p12-p11 | 740878 | Hs.2359 |
| 152 | 161574 | FRAT2 | 1 Oq23-q24.1 | 3871545 | Hs.140720 |
| 153 | 161650 | KIM0415 | 7p22.2 | 2798872 | Hs.229950 |
| 154 | 168386 | NOLC1 | 10 | 1431819 | Hs.75337 |
| 155 | 159906 | H2AFX | 11q23.2-q23.3 | 1704168 | Hs.147097 |
| 156 | 167906 | RAE1 | 20q13.31 | 2914719 | Hs.196209 |
| 157 | 160486 | DTX2 | 7q11.23 | 1691161 | Hs.89135 |
| 158 | 160678 | MAFG | 17q25 | 2956906 | Hs.252229 |
| 159 | 159889 | FUS | 16p11.2 | 3038508 | Hs.99969 |
| 160 | 167553 | LIG1 | 19q13.2-q13.3 | 1841920 | Hs.1770 |
| 161 | 163824 | UNG | 12q23-q24.1 | 1405652 | Hs.78853 |
| 162 | 161012 | GCN1 L 1 | 12q24.2 | 1699149 | Hs.75354 |
| 163 | 162006 | REG1B | 2p12 | 2374294 | Hs.4158 |
| 164 | 161454 | SPINT1 | 15q13.3 | 2722572 | Hs.233950 |
| 165 | 162510 | CAMKK2 | 12 | 557451 | Hs.108708 |
| 166 | 163306 | BLM | 15q26.1 | 2923082 | Hs.36820 |
| 167 | 160242 | RN UT1 | | 1562658 | Hs.21577 |
| 168 | 164106 | GRWD | 19q13.33 | 1561867 | Hs.218842 |
| 169 | 165799 | MADH3 | 15q21-q22 | 1858365 | Hs.211578 |
| 170 | 166574 | SNAPC2 | 19p13.3-p13.2 | 1445203 | Hs.78403 |
| 171 | 160441 | LTBR | 12p13 | 899102 | Hs.1116 |
| 172 | 168453 | TACC3 | 4p16.3 | 2056642 | Hs.104019 |
| | | | 19q13.11- | | |
| 173 | 164244 | PSMC4 | q13.13 | 2806778 | Hs.211594 |
| 174 | 169564 | SMARCD2 | 17q23-q24 | 1890919 | Hs.250581 |
| 175 | 161178 | BSG | 19p13.3 | 2182907 | Hs.74631 |
| 176 | 165614 | JUP | 17g21 | 820580 | Hs.2340 |
| 177 | 168987 | HRMT1L2 | 19q13.3 | 2888814 | Hs.20521 |
| 178 | 167987 | ENTPD1 | 10q24 | 1672749 | Hs.205353 |
| 179 | 163726 | C3 | 19p13.3-p13.2 | 1513989 | Hs.284394 |
| 180 | 164642 | YARS | 1p34.3 | 1559756 | Hs.239307 |
| 181 | 160303 | ERF | 19q13 | 2057547 | Hs.333069 |
| 182 | 161635 | TYMSTR | 3p21 | 2610374 | Hs.34526 |
| 183 | 159859 | GS2NA | 14g13-q21 | 1339241 | Hs.183105 |
| 184 | 161051 | MARK3 | 14q32.3 | 2395018 | Hs.172766 |
| | | | 1p36.11- | | |
| 185 | 161835 | PEX10 | 1p36.33 | 3115936 | Hs.247220 |
| 186 | 165571 | ANXA3 | 4q13-q22 | 1920650 | Hs.1378 |
| 187 | 164286 | NFKBIE | 6p21.1 | 2748942 | Hs.91640 |
| 188 | 165786 | HY AL2 | 3p21.3 | 1240748 | Hs.76873 |
| 189 | 161620 | H4FE | 6p22-p21.3 | 3728255 | Hs.278483 |
| 190 | 168302 | TIP-1 | 17p13 | 1997792 | Hs.12956 |
| 191 | 160887 | PES1 | 22q12.1 | 2758740 | Hs.13501 |
| 192 | 162419 | RAE1 | 20q13.31 | 588157 | Hs.196209 |
| 193 | 169625 | RFC4 | 3q27 | 1773638 | Hs.35120 |
| 194 | 163425 | TCEA2 | 20 | 818568 | Hs.80598 |
| 195 | 166359 | TUBB | 6p21.3 | 3334367 | Hs.336780 |
| 196 | 161947 | TIM17B | Xp11.23 | 1727491 | Hs.19105 |
| 197 | 162236 | KIM0670 | 14q11.1 | 1968610 | Hs.227133 |
| 198 | 168426 | RTVP1 | 12q15 | 477045 | Hs.64639 |

Classifier genes for pulmonary NET grades. To identify the classifier genes for each tumor subtype independent of the reference cell line, BEAS-2B, two-by-two comparisons are conducted on relative expression ratios in the 198 genes between three tumor subtypes. Of 198 genes, 178 show at least a 2.5-fold or highter differential expression between at least one pair of the comparisons including TC/LCNEC, TC/SCLC, LCNEC/TC, LCNEC/SCLC, SCLC/TC, and SCLC/LCNEC. Using the criteria that the expression of a gene in any one subtype is higher than those in the other two, 48 genes are identified including five in TC, seven in LCNEC and 36 in SCLC. Each group of the classifier genes can distinguish one tumor subtype from the other two. Table 7 lists the expression ratios of 48 classifier genes along with major function, chromosome location, known cytogenetic alteration and UniGene Cluster number.

TABLE 7
Expression Ratios of 48 Classifier Genes between TC, LCNEC (LC) and SCLC (SC)

| | Gene | Expression | | | | Cytogenetic | |
|---|---|---|---|---|---|---|---|
| No. | symbol | Ratio | | Function | Map | Alteration | UG cluster |
| | | TC/SC | TC/LC | | | | |
| 1 | C5 | 5.6 | 7.5 | Immune | 9q32-q34 | | Hs.1281 |
| 2 | CPE | 6.3 | 4.2 | Blosynthesis | 4q32.3 | Yes | Hs.75360 |
| 3 | GRIA2 | 5.5 | 4.0 | Receptor | 4q32-q33 | Yes | Hs.89582 |
| 4 | RIMS2 | 3.1 | 2.6 | Synaptic exocytosis | 8q23.1 | | Hs.153610 |
| 5 | ORC4L | 2.7 | 3.2 | DNA replication | 2q22-q23 | Yes | Hs.55055 |
| | | LC/TC | LC/SC | | | | |
| 1 | CSF2RB | 3.8 | 4.2 | Receptor | 22q13.1 | Yes | Hs.285401 |
| 2 | GGH | 4.8 | 6.3 | Drug resistance | 8q12.1 | Yes | Hs.78619 |
| 3 | NPAT | 2.5 | 3.8 | Cell cycle | 11q22-q23 | | Hs.89385 |
| 4 | NR3C1 | 3.8 | 5.7 | Transcription factor | 5q31 | Yes | Hs.75772 |
| 5 | P311 | 5.6 | 7.1 | Transformation | 5q22.2 | Yes | Hs.413760 |
| 6 | PRKAA2 | 2.8 | 4.2 | Metabolism | 1p31 | | Hs.2329 |
| 7 | PTK6 | 2.7 | 3.6 | Oncogene | 20q13.3 | Yes | Hs.51133 |
| | | SC/TC | SC/LC | | | | |
| 1 | APRT | 5.1 | 2.9 | Metabolism | 16q24 | | Hs.28914 |
| 2 | ARF4L | 5.4 | 3.8 | Protein secretion | 17q12-q21 | | Hs.183153 |
| 3 | ARHGDIA | 3.7 | 2.5 | RAS gene family | 17q25.3 | | Hs.159161 |
| 4 | ARL7 | 4.8 | 3.0 | Endocytosis | 2q37.2 | | Hs.111554 |
| 5 | A TP6F | 4.1 | 3.3 | Proton transport | 1p32.3 | | Hs.7476 |
| 6 | CDC20 | 7.5 | 3.3 | Cell Cycle, G 1 | 1p34.1 | Yes | Hs.82906 |
| 7 | CDC34 | 5.5 | 2.8 | Cell Cycle, G2 | 19p13.3 | Yes | Hs.423615 |
| 8 | CLDN11 | 6.2 | 2.9 | Tight junction | 3q26.2-q26.3 | Yes | Hs.31595 |
| 9 | COMT | 3.3 | 2.6 | Neurotransmission | 22q11.21 | Yes | Hs.2400 13 |
| 10 | CSTF1 | 2.8 | 2.6 | Polyadenylation | 20q13.2 | | Hs.172865 |
| 11 | DDX28 | 4.8 | 3.1 | RNA helicase | 16q22.1 | Yes | Hs.155049 |
| 12 | DHCR7 | 5.6 | 2.8 | Metabolism | 11q12-q13 | | Hs.11806 |
| 13 | ERP70 | 4.7 | 2.7 | Metabolism | 7q35 | | Hs.93659 |
| 14 | FEN 1 | 6.5 | 3.4 | Endonuclease | 11q12 | Yes | Hs.4756 |
| 15 | GCN1L1 | 3.7 | 2.6 | Translation | 12q24.2 | | Hs.75354 |
| 16 | GNB1 | 3.3 | 2.9 | Signal transduction | 1p36.33 | | Hs.215595 |
| 17 | GUK1 | 6.6 | 2.9 | Signal transduction | 1q32-q41 | | Hs.3764 |
| 18 | HDAC7A | 4.1 | 2.8 | Cell cycle, chromatin | 12q13.1 | | Hs.275438 |
| 19 | ITPA | 4.8 | 2.8 | Metabolism | 20p | | Hs.6817 |
| 20 | JUP | 4.1 | 2.6 | Cell adhesion | 17q21 | Yes | Hs.2340 |
| 21 | KIAA0469 | 4.5 | 2.8 | | 1p36.23 | | Hs.7764 |
| 22 | KRT5 | 5.7 | 3.5 | Intermediate filaments | 12q12-q13 | Yes | Hs.433845 |
| 23 | PDAP1 | 4.3 | 3.0 | Growth factor | 7q22.1 | Yes | Hs.278426 |
| 24 | PGAM1 | 4.4 | 3.1 | Metabolism | IOq25.3 | Yes | Hs.181013 |
| 25 | PHB | 4.9 | 2.8 | Antiproliferation | 17q21 | Yes | Hs.75323 |
| 26 | POLA2 | 4.7 | 2.6 | RNA synthesis | 11q13.1 | Yes | Hs.81942 |
| 27 | POLD2 | 3.7 | 2.6 | DNA replication | 7p13 | Yes | Hs.74598 |
| 28 | POLE3 | 5.5 | 3.5 | Histone-fold | 9q33 | Yes | Hs.108112 |
| 29 | PYCR1 | 4.5 | 2.6 | Metabolism | 17q24 | | Hs.79217 |
| 30 | SIP2-28 | 5.2 | 2.9 | Receptor | 15q25.3-q26 | Yes | Hs.10803 |
| 31 | SIVA | 6.5 | 3.6 | Apoptosis | 14q32.33 | | Hs.112058 |
| 32 | SURF 1 | 3.8 | 2.5 | Neurologic disorder | 9q33-q34 | | Hs.423854 |
| 33 | TADA3L | 2.8 | 2.5 | P53 cofactor | 3p25.2 | Yes | Hs.158196 |
| 34 | TKI | 4.8 | 2.7 | Metabolism | 17q25.2-q25.3 | | Hs.105097 |
| 35 | TMSTR | 3.0 | 2.5 | Signal transduction | 3p21 | | Hs.34526 |
| 36 | VATI | 4.5 | 3.4 | Neurotransmission | 17q21 | Yes | Hs.157236 |

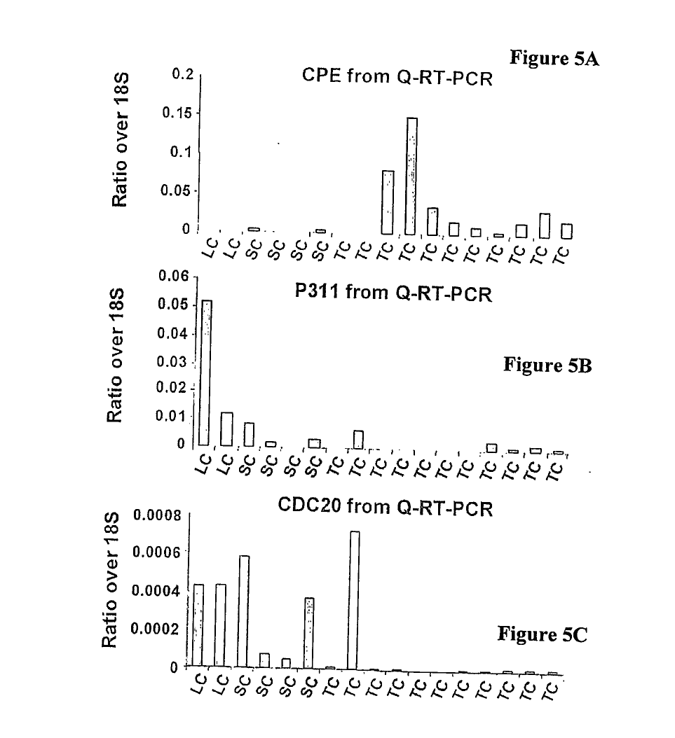

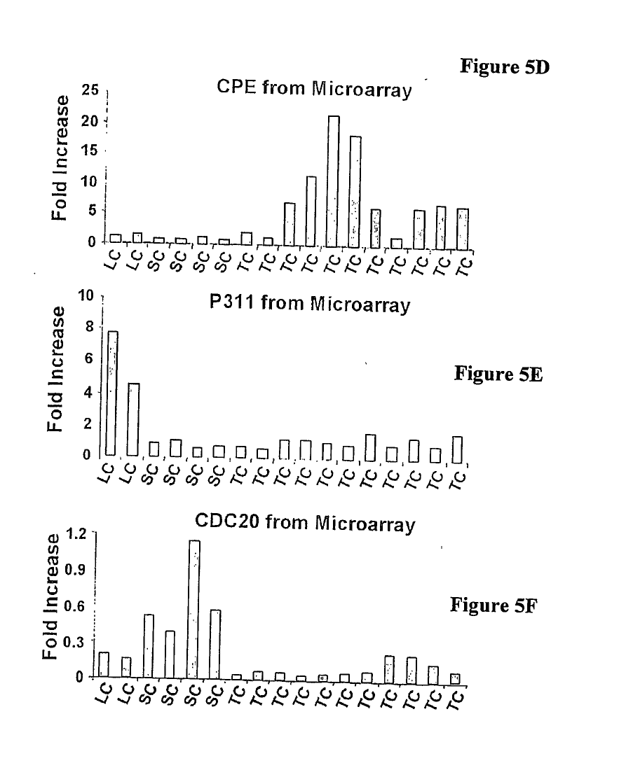

Validation of gene expression changes by real-time quantitative RT-PCR. To validate the gene expression profile and the classifier genes, real-time RT-PCR analysis are performed on three classifier genes in the 17 pulmonary NET using RNA extracted from tumor cells collected by LCM. One gene from each tumor subtype is picked based on highly differential expression for the confirmation. The expression of CPE, P311 and CDC20 detected by real-time quantitative RT-PCR in each of 17 pulmonary NET is first normalized as a ratio to the control gene 18S RNA in that tumor and then compared with the expression in the reference BEAS-2B cell line. The results show that the expression changes of these genes were highly consistent between those detected by the two methods (FIGS. 5A-5F).

Correlation of CPE and GGH protein expression to pulmonary NET grades. To initiate the identification of protein markers for analysis of archived pulmonary NET tissue sections, anti-CPE and anti-GGH antibodies are used to detect CPE and OGH expression on 68 available pulmonary NET samples including 17 used in the microarray analysis, and generated informative data on 55 cases. The images stained by anti-CPE antibody on the normal lung tissue sections, TC, LCNEC and SCLC were studied. No signal is detected in bronchial epithelial cells or pneumocytes of normal lung. Some strong staining appears in scattered neuroendocrine cells of terminal bronchiolar epithelia and in some macrophages. The TC sample displays a positive stain with strong and uniform signals on the cell membrane. The LCNEC section have a very weak and scattered anti-CPE stain, and the SCLC are completely negative. Only occasional tumor cells exhibit a weak intracytoplasmic stain. The images obtained by staining with anti-GGH antibody were also studied. Normal lung showed negative staining. TC cells also exhibited negative staining. The tumor cells have no detectable signals and mild staining can be seen only in scattered stromal cells. LCNEC cells stained positively. All tumor cells show intracytoplasmic stain, with most staining seen in the cytoplasm with a course granular staining pattern. SCLC cells show intracytoplasmic stain with course granular pattern.

Table 8 summarizes the results of anti-CPE and anti-GGH stains on the 55 pulmonary NET samples. The statistical analysis is conducted, based on the binomial distributions of positives and negatives. Of 21 cases of TC, 16 (76%) were positive to anti-CPE stain and five (24%) are negative. The difference is statistically significant (p-value <0.05). The anti-GGH stains on 21 cases of TC revealed seven positive (33%) and 14 (67%) negative, but there is no statistical significance (pvalue>0.05). Four of five (80%) AC cases are positive to the anti-CPE stain and all of the five (100%) cases are negative to the anti-GGH, but this apparent difference is not statistically significant (p-value >0.05) in light of the small sample size (n=5). Although the negative stains of anti-CPE are dominant events for LCNEC (seven negative versus one positive, 88%) the difference has no statistical significance (p-value >0.05), probably due to the small sample size. All eight cases of LCNEC are positive to anti-GGH stains (p-value <0.01). Of 21 cases of SCLC, only four (19%) are positive to anti-CPE stain (p-value <0.01). In contrast, 16 (76%) are positive to anti-GGH stain (p-value <0.05). Therefore, positive CPE stain is associated with low- and intermediate-grade TC and AC while positive GGH stain is associated with high-grade LCNEC and SCLC.

TABLE 8
Immunochemistry on 55 Pulmonary NE Tumors

| Pulmonary | Anti-CPE IHC | | | Anti-GGH IHC | | |
|---|---|---|---|---|---|---|
| NE Tumor | Positive | Negative | p-value | Positive | Negative | p-value |
| TC | 16 | 5 | 0.017 | 7 | 14 | 0.189 |
| AC | 4 | 1 | 0.625 | 0 | 5 | 0.063 |
| LCNEC | 1 | 7 | 0.070 | 8 | 0 | 0.008 |
| SCLC | 4 | 17 | 0.007 | 16 | 5 | 0.017 |
| Total | 23 | 32 | | 31 | 24 | |

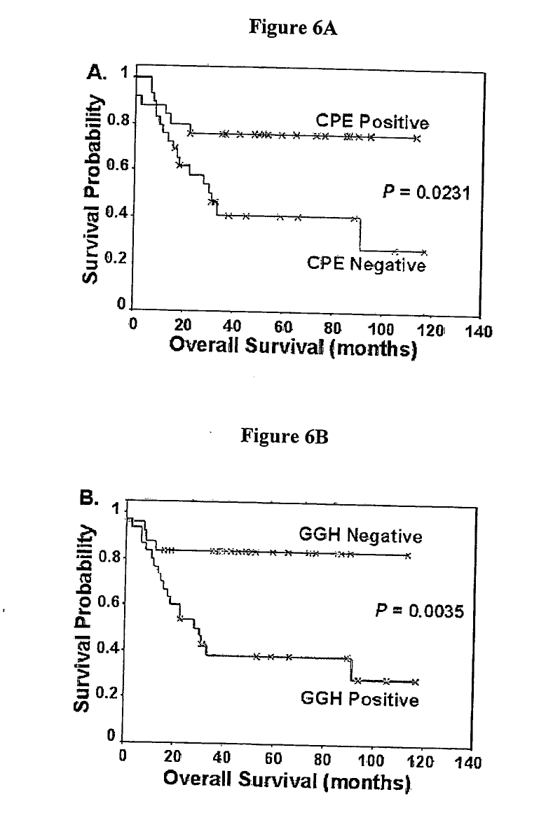

CPE and GGH protein expressions predict survival rates of the pulmonary NET patients. After the correlation of CPE and GGH expressions to pulmonary NET grades, a Kaplan-Meier survival analysis is conducted on 54 cases of the pulmonary NET patients with clinical survival data as the function of CPE or GGH stains. The 9-year survival probability for the patients with a positive CPE is 76%, significantly (p-value <0.05) higher than that with a negative CPE, 27% (FIG. 6A). In contrast, the 9-year survival probabilities for the patients with positive and negative GGH staining are 28% and 83%, respectively (FIG. 6B). The difference is statistically very significant (p-value <0.01). Thus, positive CPE and negative GGH are the good prognostic indicators for pulmonary NET patients.

In the above-described study, the expression of 9,984 genes in pulmonary NET are examined and the expression profile, 49 classifier genes and two biomarkers are identified. Homogenous cancer cells are collected by LCM from 11 cases of TC, three cases of SCLC, two cases of LCNEC and one case of combined SCLC and LCNEC. High quality RNA is extracted from the homogeneous cancer cells and subjected to T7 polymerase-based RNA amplification. cDNA microarray and unsupervised expression cluster analyses of 9,984 genes or 198 significantly (p<0.004) differentially expressed genes classified 17 cases of pulmonary NET into three groups that matched their histological classifications completely. In addition, 48 classifier genes are identified by 2-by-2 expression comparisons of 198 genes between three subtype tumors. The expression changes of representative genes are confirmed by real-time quantitative RT-PCR. Finally, based on expression profile and by IHC, it is found that positive CPE and negative GGH are more frequent events in low-grade TC and intermediate-grade AC than in high-grade LCNEC and SCLC and are good prognostic indicators for the pulmonary NET patients.

Expression clustering was developed to analyze gene expression data from DNA microarrays(Eisen, M. B. et al. (1998) “CLUSTER ANALYSIS AND DISPLAY OF GENOME-WIDE EXPRESSION PATTERNS,” Proc Natl Acad Sci USA 95:14863-14868). The analysis is based on statistical algorithms to arrange genes and tumors according to similarities in gene expression. The dendrogram is the most common output to reveal a subclass of genes and cells. In the above study, the expression pattern of 9,984 genes or selected 198 genes accurately distinguishes each subtype of 17 pulmonary NET classified by histologic characteristics. It is considered that precise LCM of the cancer cells and non-biased RNA amplification contributes to the accurate expression classification.

Luo et al.(l 999) (GENE EXPRESSION PROFILES OF LASER-CAPTURED ADJACENT NEURONAL SUBTYPES,” Nat Med 5:117-122) reported T7 polymerase-based RNA amplification (Van Gelder, R. N. et al. (1990) (“AMPLIFIED RNA SYNTHESIZED FROM LIMITED QUANTITIES OF HETEROGENEOUS cDNA,” Proc Natl Acad Sci USA 87:1663-1667) to amplify RNA isolated from LCM cells for DNA microarray study. In that case, total RNA was extracted from 1,000 neuron cells dissected by LCM and subjected to three rounds of amplification before microarray analysis, of which, the correlation of signal intensities between the same samples varied from 93% to 97% (Luo, L. et al. (1999) “GENE EXPRESSiON PROFILES OF LASER-CAPTURED ADJACENT NEURONAL SUBTYPES,” Nat Med 1999; 5:117-122). In the above-described study, total RNA is extracted from >10,000 cancer cells dissected by LCM from at least 15 sections and subjected to only two rounds of amplification. These modifications contribute to accurate clusters.

A reference sample is used as a control to normalize gene expression in test samples in cDNA microarrays. To obtain enough common RNA as a reference for all test samples is frequently difficult, particularly for a large number of primary tumors. To date, pooled normal samples or samples pooled from a portion of each test sample have been used as a reference. In this and other studies (Miura, K. et al. (2002) “LASER CAPTURE MICRODISSECTION AND MICROARRAY EXPRESSION ANALYSIS OF LUNG ADENOCARCINOMA REVEALS TOBACCO SMOKING-AND PROGNOSIS-RELATED MOLECULAR PROFILES,” Cancer Res 2002; 62:3244-3250), the RNA employed is isolated from the immortalized bronchial epithelial cell line, BEAS-2B (Reddel, R. R. et al. (1998) “TRANSFORMATION OF HUMAN BRONCHIAL EPITHELIAL CELLS BY INFECTION WITH SV40 OR ADENOVIRUS-12 SV40 HYBRID VIRUS, OR TRANSFECTION VIA STRONTIUM PHOSPHATE COPRECIPITATION WITH A PLASMID CONTAINING SV40 EARLY REGION GENES,” Cancer Res 48:1904-1909), as the reference for all test samples. Because the results demonstrated accurate classification, RNA from the cell line can be used as the reference for primary tumors. Thus, this method may be applicable to microarray analysis of gene expression of any cells where a reference sample is not easily obtained.

Using the Class Comparison analysis (or Gene Selection) of the BRB array tool, 198 genes are selected out of 9,984 genes (1.98%) for expression classification of 17 pulmonary NET. The clusters based on the 198 genes coincide well with those based on 9,984 genes. Two-by-two comparisons of 198 gene expression between the three subtypes of pulmonary NET result in the identification of 48 classifier genes of which the expression changes are able to distinguish the subtypes. The classifier genes are involved in complex regulations of apoptosis, cell-cell and cellmatrix interactions, cell cycle, DNA synthesis and repair, drug resistance, RNA synthesis and processing, and cell survival. The classifier genes provide candidates for understanding and studying pulmonary NET biology and the identification of more biomarkers.

The present invention thus provides the first report that correlates CPE and GGH expression patterns to pulmonary NET grades and prognosis. The IHC reveal patterns of CPE and GGH expression in pulmonary NET cells. Specifically, the frequency of positive staining by anti-CPE in TC (76%) is 4-fold higher than that in SCLC (19%). Although the trends of high and low frequencies of positive CPE seem apparent in AC and LCNEC, respectively, the statistical significance was not reached, perhaps due to the small sample sizes. In contrast, both LCNEC and SCLC cells displayed highly significant frequencies of positive anti-GGH stain than TC and AC cells. Significantly, the survival analysis correlates positive CPE and negative GGH on pulmonary NET cells to very good prognosis.

CPE is involved in the removal of C-terminal basic amino acids in brain and various neuroendocrine tissues. There are two types of CPE, a 50 kDa membrane-bound enzyme and a smaller soluble enzyme (Manser, E. et al. (1990) “HUMAN CARBOXYPEPTIDASE E. ISOLATION AND CHARACTERIZATION OF THE cDNA, SEQUENCE CONSERVATION, EXPRESSION AND PROCESSING IN VITRO. Biochem J 267:517-525). The former is an amphipathic and secreted enzyme (Manser, E. et al. (1991) “PROCESSING AND SECRETION OF HUMAN CARBOXYPEPTIDASE E BY C6 GLIOMA CELLS,” Biochem J 280 (Pt 3):695-701). Human CPE is located on chromosome 4p33 and no mutations are reported in lung cancers. The mutations at Ser2O2 of mouse CPE affected its expression, enzyme activity and intracellular localization (Varlamov, 0. et al. (1996) “INDUCED AND SPONTANEOUS MUTATIONS AT SER202 OF CARBOXYPEPTIDASE E. EFFECT ON ENZYME EXPRESSION, ACTIVITY, AND INTRACELLULAR ROUTING,” J Biol Chem 271:13981-13986. A mouse with Cpe/Cpe mutation results in reduced CPE enzyme activity and obesity (Naggert, J. K. et al. (1995) “HYPERPROINSULINAEMIA IN OBESE FAT/FAT MICE ASSOCIATED WITH A CARBOXYPEPTIDASE E MUTATION WHICH REDUCES ENZYME ACTIVITY,” Nat Genet 10:135-142), and as yet tumors have not been reported. The present invention shows that CPE expression is not detected in normal bronchial epithelial cells or pneumocytes; however, it is elevated in the tumor cells, suggesting that secreted CPE may be a surrogate serum marker for non-invasive diagnosis and early detection of pulmonary carcinoid tumors.

The ggh gene may be regulated at both transcriptional and posttranscriptional levels. In LCNEC cells, ggh mRNA is increased according to the microarrays, which is consistent with the increase in GGH protein based on IHC, indicating transcriptional activation. Although anti-GGH antibody detected the upregulation in three of four SCLC cases, mRNA elevation is not detected by the microarrays, suggesting an alternative posttranscriptional mechanism. The study of mechanism(s) of ggh transcription and translation is of importance, not only because it has diagnostic and prognostic value, but also because the GGH protein (as lysosomal enzyme that catalyzes the hydrolysis of folylpoly-γ-glutamates and antifolylpoly-γ-glutamates by the removal of γ-linked polyglutamates and glutamate (Wang, Y. et al. (1993) “THE PROPERTIES OF THE SECRETED GAMMA-GLUTAMYL HYDROLASES FROM H35 HEPATOMA CELLS,“Biochim Biophys Acta 1164:227-235)) are known to be implicated in methotrexate resistance in sarcoma (Waltham, M. C. et al. (1997) “GAMMA-GLUTAMYL HYDROLASE FROM HUMAN SARCOMA HT-1080 CELLS: CHARACTERIZATION AND INHIBITION BY GLUTAMINE ANTAGONISTS,” Mol Pharmacol 51:825-832; Li, W. W. (1993) “INCREASED ACTIVITY OF GAMMA-GLUTAMYL HYDROLASE IN HUMAN SARCOMA CELLLINES: A NOVEL MECHANISM OF INTRINSIC RESISTANCE TO METHOTREXATE (MTX),” Adv Exp Med Biol 338:635-638) and leukemia (Longo, G. S. et al. (1997) “GAMMA GLUTAMYL HYDROLASE AND FOLYLPOLYGLUTAMATE SYNTHETASE ACTIVITIES PREDICT POLYGLUTAMYLATION OF METHOTREXATE IN ACUTE LEUKEMIAS,” Oncol Res 9:259-263; Rots, M. G. et al. (1999) “ROLE OF FOLYLPOLYGLUTAMATE SYNTHETASE AND FOLYLPOLYGLUTAMATE HYDROLASE IN METHOTREXATE ACCUMULATION AND POLYGLUTAMYLATION IN CHILDHOOD LEUKEMIA,” Blood 93:1677-1683).

In sum, pulmonary neuroendocrine tumors are found to vary dramatically in their malignant behavior and classification based on histological examination is often challenging. In searching for molecular markers for these tumors, a cDNA microarray expression analysis is conducted. The analysis involved 9,984 genes in tumor cells isolated by laser-capture microdissection from primary tumors of typical carcinoids (TC), small cell lung cancers (SCLC), large cell neuroendocrine carcinomas (LCNEC), and a combined small cell and large cell neuroendocrine carcinoma. An unsupervised, hierarchical clustering algorithm resulted in a precise classification of each tumor subtype, according to the newly proposed, modified histological classification. Selection of genes with significant variance resulted in the identification of 198 statistically significant genes (p<0.004) that accurately discriminated between three predefined tumor subtypes. Of 198 genes, 48 classifier genes are identified. Changes in expression of three representative, differentially expressed genes were internally validated by real-time RT-PCR. In addition, expression of two classifier gene products, carboxypeptidase E (CPE) and γ-glutamyl hydrolase (GGH), are validated by immunohistochemistry. Kaplan-Meier survival analysis reveals that CPE immunostaining is a statistically significant predictor of good prognosis, whereas GGH expression correlated with poor prognosis. Thus, this molecular profiling accurately classifies pulmonary neuroendocrine tumors and permits the identification of 48 classifier genes and two novel prognostic markers.

All publications and patents mentioned in this specification are herein incorporated by reference to the same extent as if each individual publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method for determining whether a candidate cell is a neuro-endocrine tumor cell, wherein said method comprises the steps of:
(A) determining the profile of expression of a plurality of genes of said candidate cell; and
(B) comparing such determined profile of expression with the profile of expression of said genes of a small cell lung cancer cell, a large cell neuroendocrine carcinoma cell, a typical carcinoid tumor cell or an atypical carcinoid tumor cell;
to thereby determine whether said candidate cell is a neuroendocrine tumor cell.

2. The method of claim 1, wherein said method additionally permits a determination of neuroendocrine tumor cell type.

3. The method of claim 2, wherein said method determines whether said candidate cell is a small cell lung cancer (SCLC) neuroendocrine tumor cell.

4. The method of claim 1, wherein said plurality of genes includes one or more genes selected from the group consisting of C5, CPE, GRIA2, RIMS2, ORC4L, CSF2RB, GGH, NPAT, NR3C1, P311, PRKAA2, PTK6, APRT, ARF4L, ARHGD1A, ARL7, ATP6F, CDC20, CDC34, CLDN11, COMT, CSTF11, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCR1, SIP2-28, SIVA, SURF1, TADA3L, TK1, TYMSTR, and VATI.

5. The method of claim 4, wherein said plurality of genes includes one or more genes selected from the group consisting of GGH and CPE.

6. The method of claim 2, wherein said method determines whether said candidate cell is a large cell neuroendocrine carcinoma (LCNEC) neuroendocrine tumor cell.

7. The method of claim 2, wherein said method determines whether said candidate cell is a typical carcinoid (TC) neuroendocrine tumor cell.

8. The method of claim 2, wherein said method determines whether said candidate cell is an atypical carcinoid (AC) neuroendocrine tumor cell.

9. The method of claim 2, wherein said step (A) comprises incubating RNA of said candidate cell, or DNA or RNA amplified from such RNA, in the presence of a plurality of genes, or fragments or RNA transcripts thereof, under conditions sufficient to cause RNA to hybridize to complementary DNA or RNA molecules; and detecting hybridization that occurs.

10. The method of claim 9, wherein said plurality of genes, or polynucleotide fragments or RNA transcripts thereof, are distinguishably arrayed in a microarray.

11. The method of claim 10, wherein said microarray comprises arrayed genes, or polynucleotide fragments or RNA transcripts thereof, that are differentially expressed in-neuroendocrine tumor cells relative to normal cells.

12. The method of claim 10, wherein said microarray comprises arrayed genes, or polynucleotide fragments or RNA transcripts thereof, that are differentially expressed in small cell lung cancer (SCLC) neuroendocrine tumor cells relative to large cell neuroendocrine carcinoma (LCNEC) neuroendocrine tumor cells.

13. The method of claim 12, wherein said arrayed genes, or polynucleotide fragments or RNA transcripts thereof, include one or more genes selected from the group consisting of C5, CPE, GRIA2, RlMS2, ORC4L, CSF2RB, GGH, NPAT, NR3C1, P311, PRKAA2, PTK6, APRT, ARF4L, ARHGD1A, ARL7, ATP6F, CDC20, CDC34, CLDN11, COMT, CSTF1, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCRI, SIP2-28, SIVA, SURF 1, TADA3L, TK1, TYMSTR, and VATI, or a polynucleotide fragment or RNA transcript thereof.

14. The method of claim 13, wherein said arrayed genes, or polynucleotide fragments or RNA transcripts thereof, includes one or more genes selected from the group consisting of GGH and CPE, or a polynucleotide fragment or RNA transcript thereof.

15. The method of claim 10, wherein said microarray comprises arrayed genes, or polynucleotide fragments or RNA transcripts thereof, that are differentially expressed in small cell lung cancer (SCLC) neuroendocrine tumor cells relative to typical carcinoid (TC) neuroendocrine tumor cells.

16. The method of claim 10, wherein said microarray comprises arrayed genes, or polynucleotide fragments or RNA transcripts thereof, that are differentially expressed in small cell lung cancer (SCLC) neuroendocrine tumor cells relative to atypical carcinoid (AC) neuroendocrine tumor cells.

17. The method of claim 10, wherein said microarray comprises arrayed genes, or polynucleotide fragments or RNA transcripts thereof, that are differentially expressed in large cell neuroendocrine carcinoma (LCNEC) neuroendocrine tumor cells relative to typical carcinoid (TC) neuroendocrine tumor cells.

18. The method of claim 10, wherein said microarray comprises arrayed genes, or polynucleotide fragments or RNA transcripts thereof, that are differentially expressed in large cell neuroendocrine carcinoma (LCNEC) neuroendocrine tumor cells relative to atypical carcinoid (AC) neuroendocrine tumor cells.

19. The method of claim 10, wherein said microarray comprises arrayed genes, or polynucleotide fragments or RNA transcripts thereof, that are differentially expressed in typical carcinoid (TC) neuroendocrine tumor cells relative to atypical carcinoid (AC) neuroendocrine tumor cells.

20. A microarray of genes, or polynucleotide fragments or RNA transcripts thereof for distinguishing a neuroendocrine tumor cell, said microarray comprising a solid support having greater than 10 genes, or polynucleotide fragments or RNA transcripts thereof, distinguishably arrayed in spaced apart regions, wherein said microarray comprises a sufficient number of genes, or polynucleotide fragments or RNA transcripts thereof, that are differentially expressed in a small cell lung cancer (SCLC) cell, a large cell neuroendocrine carcinoma (LCNEC) neuroendocrine tumor cell, a typical carcinoid (TC) neuroendocrine tumor cell, or an atypical carcinoid (AC) neuroendocrine tumor cell, relative to a normal cell or a cell belonging to a different neuroendocrine tumor cell type, to permit said microarray to distinguish a neuroendocrine tumor cell.

21. The microarray of claim 20, wherein said microarray comprises a sufficient number of genes, or polynucleotide fragments or RNA transcripts thereof, that are differentially expressed in a neuroendocrine tumor cell relative to a normal cell to permit said microarray to distinguish between a neuroendocrine tumor cell and a normal cell.

22. The microarray of claim 20, wherein said microarray comprises a sufficient number of genes, or polynucleotide fragments or RNA transcripts thereof, that are differentially expressed in a small cell lung cancer (SCLC) neuroendocrine tumor cell relative to a large cell neuroendocrine carcinoma (LCNEC) neuroendocrine tumor cell to permit said microarray to distinguish between a small cell lung cancer (SCLC) neuroendocrine tumor cell and a large cell neuroendocrine carcinoma (LCNEC) neuroendocrine tumor cell.

23. The microarray of claim 20, wherein said microarray comprises a sufficient number of genes, or polynucleotide fragments or RNA transcripts thereof, that are differentially expressed in a small cell lung cancer (SCLC) neuroendocrine tumor cell relative to a typical carcinoid (TC) neuroendocrine tumor cell to permit said microarray to distinguish between a small cell lung cancer (SCLC) neuroendocrine tumor cell and a typical carcinoid (TC) neuroendocrine tumor cell.

24. The microarray of claim 20, wherein said microarray comprises a sufficient number of genes, or polynucleotide fragments or RNA transcripts thereof, that are differentially expressed in a small cell lung cancer (SCLC) neuroendocrine tumor cell relative to an atypical carcinoid (AC) neuroendocrine tumor cell to permit said microarray to distinguish between a small cell lung cancer (SCLC) neuroendocrine tumor cell and an atypical carcinoid (AC) neuroendocrine tumor cell.

25. The microarray of claim 20, wherein said microarray comprises a sufficient number of genes, or polynucleotide fragments or RNA transcripts thereof, that are differentially expressed in a large cell neuroendocrine carcinoma (LCNEC) neuroendocrine tumor cell relative to a typical carcinoid (TC) neuroendocrine tumor cell to permit said microarray to distinguish between a large cell neuroendocrine carcinoma (LCNEC) neuroendocrine tumor cell and a typical carcinoid (TC) neuroendocrine tumor cell.

26. The microarray of claim 20, wherein said microarray comprises a sufficient number of genes, or polynucleotide fragments or RNA transcripts thereof, that are differentially expressed in a large cell neuroendocrine darcinoma (LCNEC) neuroendocrine tumor cell relative to an atypical carcinoid (AC) neuroendocrine tumor cell to permit said microarray to distinguish between a large cell neuroendocrine carcinoma (LCNEC) neuroendocrine tumor cell and an atypical carcinoid (AC) neuroendocrine tumor cell.

27. The microarray of claim 20, wherein said microarray comprises a sufficient number of genes, or polynucleotide fragments or RNA transcripts thereof, that are differentially expressed in a typical carcinoid (TC) neuroendocrine tumor cell relative to an atypical carcinoid (AC) neuroendocrine tumor cell to permit said microarray to distinguish between a typical carcinoid (TC) neuroendocrine tumor cell and an atypical carcinoid (AC) neuroendocrine tumor cell.

28. The microarray of claim 20, wherein said genes or polynucleotide fragments or RNA transcripts thereof of said microarray include one or more genes selected from the group consisting of C5, CPE, GRIA2, RIMS2, ORC4L, CSF2RB, GGH, NPAT, NR3C1, P311, PRKAA2, PTK6, APRT, ARF4L, ARHGDIA, ARL7, ATP6F, CDC20, CDC34, CLDN11, COMT, CSTF1, DDX28, DHCR7, ERP70, FEN1, GCN1L1, GNB1, GUK1, HDAC7A, ITPA, JUP, KIAA0469, KRT5, PDAP1, PGAM1, PHB, POLA2, POLD2, POLE3, PYCRI, SIP2-28, SIVA, SURF1, TADA3L, TK1, TYMSTR, and VATI, or a polynucleotide fragment or RNA transcript thereof.

29. The method of claim 28, wherein said genes or polynucleotide fragments or RNA transcripts thereof of said microarray include one or more genes selected from the group consisting of GGH and CPE, or a polynucleotide fragment or RNA transcript thereof.


SEQUENCE LISTING (7 sequences)

SEQ ID NO: 1 - DNA, 66 bp, Artificial Sequence
  Description of Artificial Sequence Synthetic oligonucleotide
tctagtcgacggccagtgaattgtaatacgactcactatagggcgttttttttttttttttttttt

SEQ ID NO: 2 - DNA, 23 bp, Artificial Sequence
  Description of Artificial Sequence Synthetic primer
ttgtccgagaccttcaaggtaac

SEQ ID NO: 3 - DNA, 21 bp, Artificial Sequence
  Description of Artificial Sequence Synthetic primer
cctttgcggatgtaacatcgt

SEQ ID NO: 4 - DNA, 22 bp, Artificial Sequence
  Description of Artificial Sequence Synthetic primer
tgggtcagtcaagaaccatttc

SEQ ID NO: 5 - DNA, 23 bp, Artificial Sequence
  Description of Artificial Sequence Synthetic primer
acttcctttgggacaggaagtct

SEQ ID NO: 6 - DNA, 24 bp, Artificial Sequence
  Description of Artificial Sequence Synthetic primer
ctgaacggttttgatgtagaggaa

SEQ ID NO: 7 - DNA, 18 bp, Artificial Sequence
  Description of Artificial Sequence Synthetic primer
ccctctggcgcattttgt